(19) United States
(12) Patent Application Publication
(10) Pub. No.: US 2017/0319184 A1
(43) Pub. Date: Nov. 9, 2017

(54) HEALTH CARE SYSTEM

(71) Applicant: Nomura Research Institute, Ltd., Tokyo (JP)

(72) Inventor: Noriko SANO, Tokyo (JP)

(73) Assignee: Nomura Research Institute, Ltd., Tokyo (JP)

(21) Appl. No.: 15/654,452

(22) Filed: Jul. 19, 2017

Related U.S. Application Data
(63) Continuation of application No. PCT/JP2016/052416, filed on Jan. 28, 2016.

(30) Foreign Application Priority Data
Jan. 28, 2015 (JP) 2015-014406

(51) Int. Cl. A61B 10/00; A61B 5/01; A61B 5/00; G06F 19/00

(52) U.S. Cl. CPC A61B 10/0012; A61B 5/7278; A61B 5/01; A61B 5/43; A61B 5/742; A61B 5/0022; A61B 5/7275; A61B 5/486; A61B 2010/0019; G06F 19/322; G06F 19/3431

(57) ABSTRACT

Heath care system has a server providing service for caring for the health state of each user, and a terminal of the user. The server registers and manages health information including examination results, body temperature and menstruation data of each user based on operation from the user terminal, determines the health state of each user, including a tendency of variation of values of an examination item, based on comparison of time series values of the examination item of the examination result data and based on comparison result between the values of the examination item of the examination result data and a numerical range of reference information corresponding to the examination item, and outputs, to the user terminal, information including a graph of the examination result data, a graph of the body temperature and menstruation data, and a message appropriate for the health state of each user.

### TECHNICAL FIELD

The present invention relates to a service technique by information processing. The present invention relates to a health care technique which cares for physical and mental states (generically referred to as a health state) including health, illness, symptoms, and the like of human beings. The present invention relates to a technique which supports use of medical care and examination of human beings (including a patient). The present invention relates to a supporting technique for maintenance and improvement of the health states. The present invention relates to an information processing technique concerned with obstetrics and gynecology which deal with women's diseases, pregnancy and childbirth, and reproductive medicine.

### BACKGROUND ART

Demand for information processing service relating to support of health care and medical use has been increasing. For example, a large number of men and women are currently concerned about pregnancy and childbirth related to women's diseases and the couples' fertility. Since the number of female eggs decreases with age and the eggs also age, possibility of pregnancy becomes lower and the pregnancy carries a higher risk at older age. Moreover, it has been studied that male sperm motility also decreases with age. It is effective and important to consciously work on pregnancy, which is the result of joint activity of a man and a woman, from young age. Early treatment and the like are effective and important for infertility. Besides infertility, the women's diseases include premenstrual syndrome (PMS), menopausal disorder, corpus luteum insufficiency, endometriosis, and the like. Also, diseases specific to men include oligozoospermia and the like affecting infertility.

As a technique for caring for the above health states of women, there is service which inputs and records, in a server, basal body temperature data of a user from an application of a terminal, displays the body temperature data on a screen, and provides the user with general medical knowledge of a menstrual cycle and the like and advice on daily life.

Related-art examples concerned with management of individual medical condition include National Publication of International Patent Application No. 2011-501844 (Patent Document 1). Patent Document 1 describes that, on a screen, medical condition evaluation indexes and information on interventions such as medicine administration are input by a patient, who is an individual user, and displayed with line graphs. The medical condition evaluation indexes indicate qualitative values of mood and the like and quantitative values of blood pressure, body temperature, and the like. The interventions indicate activities such as treatment, medicine, diet, and exercise relevant to the medical condition. Patent Document 1 is a technology for observing the state of influence of the patient's actions such as taking medicine on his or her medical condition.

### RELATED ART DOCUMENT

### Patent Document

National Publication of International Patent Application No. 2011-501844

### SUMMARY OF THE INVENTION

### Problems to be Solved by the Invention

Conventional techniques relating to women's diseases and pregnancy support have the following problems. (1) The medical information provided is general commentary, enlightenment information, and the like and thus is insufficient, and it is difficult to interpret the health state and medical information of the user, including body temperature, examination results, and the like. (2) It takes time and effort to input data of the health state of the user, and it is difficult to keep a regular history, so that it is difficult to analyze the self-health information. (3) As for the pregnancy support, support for activities of male and female partners is insufficient. The conventional service is functionally insufficient for health care especially in the field including female pregnancy and infertility.

(1) The information provided by the conventional service is on illness, drugs, perspective on basal body temperature, and explanation of an ovulation day and is information uniformly enlightening all users. Moreover, conventionally, the user only receives the examination result paper for the examination results, and there has been no service which enables the user to know the details of the examination results, the relations among the examination items, information appropriate for the female hormone values and the like of the examination results, his or her current health state based on the medical information, and the like.

Therefore, it is difficult for the user to understand how to interpret and judge the values of the body temperature and the examination results and other relevant medical information, which relate to his or her health state and the contents of the treatment and the examination. Moreover, it is difficult for the user to judge what kind of treatment and examination should be taken and what kind of actions such as exercise and diet, should be taken to maintain or improve his or her health state. For example, regarding limited information from the contacted gynecology department, obstetrics and gynecology department and hospital specialized in in-vitro fertilization, the user has difficulty understanding and is concerned about the health state including his or her body temperature and menstruation (also called menstrual period), states of female hormones, possibility of pregnancy or infertility, condition and meaning of medication, possibility of specific diseases, and the like.

Regarding the above, the user conventionally exchanges body temperature, examination results, symptoms, medical information, and the like on the Internet bulletin boards and the like. For example, topics are female hormone values of blood test results, the results of the determination as to whether the values are normal or not, and the like. However, these pieces of information are prosaic, making it difficult to judge and acquire necessary information for each user. Since it is difficult for the user to understand the medical information, for example, there are cases where the user is misled by comparing values resulted from different examination methods, and the like, without recognizing that different examination institutions have different examination methods and different reference information for judging the examination values. The user easily gets confused about how to judge especially when each medical institution has different contents and ideas for treatment and each examination institution has different examination methods, and the like.

(2) To analyze the health state, not only comparison with reference information provided by the examination institutions, but also analysis of variation of the examination values of the user is important in some cases. However, input of data such as body temperature by the user generally takes time and effort and is troublesome. Therefore, it is difficult for the user to have motivation and willingness to continuously register the data. Moreover, it is difficult for the user to recognize and grasp the influences and the results of the treatment and the actions actually taken to improve the health state of the user, recover the medical condition, and the like. This point also relates to the difficulty having the above motivation and willingness. Furthermore, when the user regularly visits a hospital for pregnancy, the examination contents are often kept by a paper medium, and thus, it is difficult to regularly accumulate and search the information. For this reason, it is difficult to perform time series analysis and analysis among pieces of information such as relations between the examination results, symptoms, and the like, and the actions.

(3) It is effective to work on pregnancy activities, treatment, and the like by male and female partners, especially in the field relating to the pregnancy support. However, conventionally, there has been no service which supports the activities by the male and female partners.

An object of the present invention is to provide techniques which can achieve, regarding the techniques of the above health care and the like, support for interpretation and acquisition of the user's health state and medical information, richness and enhancement of the provided information on the user's health state and medical information, reduction in time and effort for data input by the user, support for activities by male and female partners, and the like, and which can thus comprehensively care for the health state of the user and support the treatment and the examination.

### Means for Solving the Problems

A representative embodiment of the present invention is a health care system which provides an information processing service that cares for a health state of a user, and the health care system has the following configuration.

(1) A health care system according to one embodiment includes:

a server device providing service for caring for a health state of each user; and

a terminal of the user,

in which the server device includes:
a data management unit registering and managing health information including examination result data of each user based on operation from the terminal of the user;
an analysis unit determining the health state of each user, including a tendency of variation of values of an examination item, based on comparison between a past value and a current value of time series values of the examination item of the examination result data and based on comparison result between the time series values of the examination item of the examination result data and a numerical range of reference information corresponding to the examination item; and
an output unit outputting, to the terminal of the user, information including a time series graph of the examination result data and a message appropriate for the health state of each user.

### Effects of the Invention

According to the representative embodiment of the present invention, regarding the techniques of the above health care and the like, it is possible to achieve support for interpretation and acquisition of the user's health state and medical information, richness and enhancement of provided information on the user's health state and medical information, reduction in time and effort for data input by the user, support for activities by male and female partners, and the like, and it is thus possible to comprehensively care for the health state of the user and support the treatment and the examination.

### BRIEF DESCRIPTIONS OF THE DRAWINGS

FIG. 1 is a diagram showing a configuration of a health care system according to a first embodiment of the present invention;

FIG. 2A is a diagram showing functions of the health care system and an outline of data according to the first embodiment;

FIG. 2B is a diagram showing the functions of the health care system and the outline of data according to the first embodiment;

FIG. 3 is a diagram showing a main processing flow of the health care system according to the first embodiment;

FIG. 4 is a diagram showing a configuration example of user attribute information according to the first embodiment;

FIG. 5 is a diagram showing a configuration example of examination result data according to the first embodiment;

FIG. 6 is a diagram showing a configuration example of calendar input information according to the first embodiment;

FIG. 7 is a diagram showing a configuration example of output message information according to the first embodiment;

FIG. 8 is a diagram showing a configuration example of medical examination information according to the first embodiment;

FIG. 9 is a diagram showing a specific example of the medical examination information according to the first embodiment;

FIG. 10 is a diagram showing a screen example including clinical record information according to the first embodiment;

FIG. 11 is a diagram showing a screen example including the calendar, and an input example by unit of one day according to the first embodiment;

FIG. 12 is a diagram showing a screen example of input fields of symptom information according to the first embodiment;

FIG. 13 is a diagram showing an example of a body temperature-menstruation graph according to the first embodiment;

FIG. 14 is a diagram showing a first example of an examination result graph according to the first embodiment;

FIG. 15 is a diagram showing a second example of the examination result graph according to the first embodiment;

FIG. 16 is a diagram showing an example of tendency analysis processing of the body temperature and the menstruation according to the first embodiment;

FIG. 17 is a diagram showing a flow of action extraction processing according to the first embodiment;

FIG. 18 is a diagram showing an example of the action extraction processing according to the first embodiment;

FIG. 19 is a diagram showing an example of graph interpolation and graph matching according to the first embodiment;

FIG. 20 is a diagram showing a first example of processing definition information according to the first embodiment;

FIG. 21 is a diagram showing a second example of the processing definition information according to the first embodiment;

FIG. 22 is a diagram showing a third example of the processing definition information according to the first embodiment;

FIG. 23 is a diagram showing a fourth example of the processing definition information according to the first embodiment;

FIG. 24 is a diagram showing a configuration of a health care system according to a second embodiment of the present invention;

FIG. 25 is a diagram showing a first screen example of a terminal of a female user according to the second embodiment;

FIG. 26 is a diagram showing a second screen example of the terminal of the female user according to the second embodiment;

FIG. 27 is a diagram showing a third screen example of the terminal of the female user according to the second embodiment;

FIG. 28 is a diagram showing a first screen example of a terminal of a male user according to the second embodiment; and

FIG. 29 is a diagram showing a second screen example of the terminal of the male user according to the second embodiment.

### DETAILED DESCRIPTION OF PREFERRED EMBODIMENTS

Hereinafter, a health care system according to a first embodiment of the present invention will be described in detail with reference to the drawings. As for the definitions of the terms used in this specification, disease is a generic term for so-called sickness, illness, disease, malady, syndrome, disorder, and others. The disease is managed including name, type, degree, stage, transition, details, and the like. The disease is managed including a suspected state of disease, a state of currently being ill, a state of being recovered from the illness, and the like. The disease includes one based on a diagnosis by a doctor and the like and one based on user's self-recognition and subjectivity. The disease includes especially a disease concerned with the fields of obstetrics, gynecology, and reproductive medicine, but may also include a disease of other medical fields.

Treatment is a generic term for clinical examination, treatment, medical activities, prescription, and the like by a medical institution, therapy employed by the user, and the like. The treatment is managed including name, type, stage, transition, details, and the like. Examples of the treatment include counseling, a timing method (a method of performing sexual intercourse to coincide with the ovulation day), artificial insemination, in-vitro fertilization, microinsemination, surgery of ovarian or uterus, injection of medicine, and the like.

Examination is a medical examination and a generic term for a test and the like. Examples of the examination include a blood test, a urinalysis, a semen examination, a physiological function test by ultrasound and an endoscope, an imaging examination, and the like. The examination includes a test for each specific disease such as sexually transmitted diseases and includes a general health examination.

A symptom is a generic term for an actual state of exercise, diet, sleep, excretion, and the like, mood, physical condition, and the like and may include stress. The symptom and the stress include various physical and mental symptoms and stress which are subjectively recognized by the user. An action is a generic term for exercise, diet, sleep, excretion, sexual intercourse, and other various activities in daily life, which are planned subjectively by the user for the purpose of improving the disease.

### First Embodiment

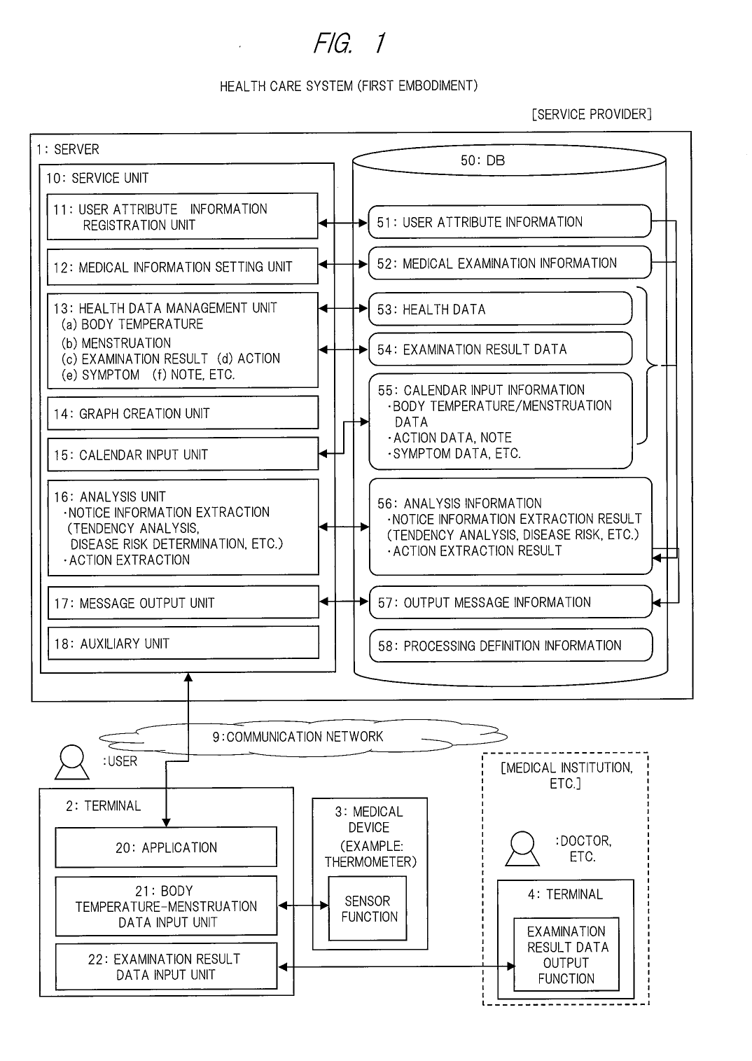

A configuration of a health care system according to the first embodiment will be described with reference to FIGS. 1 to 23. The configuration of the health care system according to the first embodiment is intended for the fields of obstetrics, gynecology, and reproductive medicine (including urology in the case of men) to provide service which cares for a health state of a user at the time of women's diseases (including symptoms accompanying increase or decrease in female hormones) and at the time of events such as pregnancy (including infertility and the like) and which supports data recording and analysis of activities including treatment and an examination of the user. This service manages health data of each individual user, analyzes the health state of each individual user, and provides information such as messages appropriate for the state of each individual user.

[System]

FIG. 1 is a diagram showing the entire configuration of the health care system according to the first embodiment of the present invention. In the configuration of the health care system according to the first embodiment, a server 1 by a service provider and a terminal 2 of each of a plurality of users are connected via a communication network 9. The user is a person including a patient or the like and owns the terminal 2 and a medical device 3. The terminal 2 of the user may be connected to a terminal 4 of a medical institution or an examination institution via the communication network 9. The server 1 may be connected to the terminal 4 of the medical institution or the examination institution via the communication network 9. Servers of other providers may be connected to the server 1 to provide service in cooperation with the server 1.

The medical institution may be a hospital or the like. The examination institution may be an examination company, an examination department in the medical institution, or the like. The servers of other providers may be servers of Web sites which provide medical information and hospital information, servers of communication carriers which manage user information and provide payment service, or the like.

The server 1 has a service unit 10 and a database (DB) 50. Based on the processing of a server program of a server computer, the service unit 10 provides the terminal 2 of the user, who has accessed via the communication network 9, with a screen and processing of health care service, using information in the DB 50. The DB 50 is configured with a storage and the like, stores data and information for the service, and is managed securely. The server 1 may be a cloud computing system or the like.

The terminal 2 of the user can be various types of computers such as a PC, a smartphone, a tablet terminal, and a mobile phone and includes known elements such as a CPU, a ROM, a RAM, an input unit, an output unit, and a communication unit. The terminal 2 of the user has an application 20, a body temperature-menstruation data input unit 21 and an examination result data input unit 22.

The application 20 is a program which performs processing to receive the health care services by communicating with the service unit 10 of the server 1 and provides a user interface including a screen of the service. The application 20 includes implementations of functions corresponding to the body temperature-menstruation data input unit 21 and the examination result data input unit 22.

The body temperature-menstruation data input unit 21 inputs body temperature data and menstruation data of the user. The body temperature data is time series data including a date and a value of measurement of basal body temperature, and the like. The menstruation data is time series data including information such as a menstruation date. The examination result data input unit 22 inputs examination result data of the user. The examination result data is time series data including an examination date, examination items, values, and the like. The examination items include an endocrinological examination and the like of female hormones and the like. Besides manual input, the body temperature-menstruation data input unit 21 and the examination result data input unit 22 can perform the input by automatic transfer, for example, are provided with a wireless communication interface to input data from an outside by wireless communication.

The medical device 3 includes a thermometer used to measure the basal body temperature by the user, an examination checker, and the like. The medical device 3 is provided with a measurement function for the body temperature and the like, as a sensor function. The medical device 3 can store, display, and externally output data of the body temperature and the like measured by the sensor function. The body temperature-menstruation data input unit 21 of the terminal 2 of the user inputs the data of the body temperature and the like from the medical device 3 by communication.

The terminal 2 of the user and the medical device 3 may be wearable terminals having the sensor function. In this case, the wearable terminal automatically measures the body temperature and values of other predetermined items concerned with the health state of the user and records the data. The terminal 2 and the medical device 3 may be integrated into one. There may be a plurality of medical devices 3 appropriate for measurement target items.

A person such as a doctor of the medical institution or an examiner of the examination institution uses the terminal 4. Moreover, the user may use the terminal 4 at home and the like. The terminal 4 may be a dedicated medical device, a dedicated examination device, hospital system, or the like, besides various types of computers like the terminal 2 of the user, or may be dedicated pharmaceuticals, a dedicated examination checker, or the like. For example, the doctor, the examiner, or the user manually inputs information on the treatment and the like of the user (so-called clinical record information) and examination result information into the terminal 4. Alternatively, when the terminal 4 is a medical device, an examination device, or a hospital system, data is automatically transferred. The terminal 4 is provided with an examination result data output function and can externally output the examination result data of the user. With the examination result data input unit 22, the terminal 2 of the user can input the examination result data from the examination result data output function of the terminal 4 via communication.

The service unit 10 has a user attribute information registration unit 11, a medical information setting unit 12, a health data management unit 13, a graph creation unit 14, a calendar input unit 15, an analysis unit 16, a message output unit 17, and an auxiliary unit 18. Each unit is realized by software program processing. The DB 50 stores user attribute information 51, medical examination information 52, health data 53, examination result data 54, calendar input information 55, analysis information 56, output message information 57, processing definition information 58, and the like.

In addition to the above, the service unit 10 includes a function of providing a basic service to the terminal 2 of the user and manages information for the processing in the DB 50. The service unit 10 acquires or refers to necessary information from the servers of other providers as appropriate and performs the processing for the basic service. The basic service provides the latest medical information and health information, searches for medical institutions, pharmaceuticals (including vitamins and Kampo medicines), and the like, has functions of bulletin boards (media such as a community where people read and write), blogs, and the like.

The user attribute information registration unit 11 provides the terminal 2 of the user with a screen for information registration and performs processing for registering, as the user attribute information 51, attribute information on the user input by the user on the screen, and processing for setting the setting information for each user.

Based on an input by an administrator of the present system, the medical information setting unit 12 performs processing for setting management information of the present system, including the medical examination information 52 and the processing definition information 58. The medical examination information 52 is management information on medical care and examination and is a DB of information on medical institutions and examination institutions. The processing definition information 58 is information which defines individual processing logic such as analysis.

The health data management unit 13 performs processing for managing, in the DB 50, as health data (also called health information), data of various elements input by the user through the application 20 of the terminal 2 of the user, that is, information such as body temperature, menstruation, examination result, action, symptom, and note, etc. In particular, the health data management unit 13 receives the body temperature data and the menstruation data input and transmitted through the body temperature-menstruation data input unit 21 of the terminal 2 and stores the data as the health data 53. The health data management unit 13 also receives the examination result data input and transmitted through the examination result data input unit 22 of the terminal 2 and stores the data as the examination result data 54.

The graph creation unit 14 performs processing for creating a body temperature-menstruation graph using the health data 53, storing the graph as a part of the health data 53, and displaying the body temperature-menstruation graph on the screen. The graph creation unit 14 also performs processing for creating an examination result graph using the examination result data 54, storing the graph as a part of the examination result data 54, and displaying the examination result graph on the screen. For example, the graph includes a graph in which a horizontal axis indicates time such as the number of days, and values of the body temperature and the like are plotted along a vertical axis. The body temperature-menstruation graph is an integrated graph of a body temperature graph and a menstruation graph, but may be managed separately. The examination result graph includes a graph of values of examination items of endocrinological examinations and the like.

The calendar input unit 15 is a processing unit which assists input and management of the health data in the health data management unit 13. The calendar input unit 15 provides the terminal 2 of the user with a screen including a calendar and performs processing for registering user input information which is input by the user on the screen and includes the basal body temperature, the menstruation, the examination result, the action, the symptom, the note, the treatment, the medication, and other information, as the calendar input information 55, regardless of a static method or a dynamic method. Information on various items of the health data can be registered in time series for each calendar date, and information on each item can be input with at least one of a dedicated screen, an input field, and a calendar.

Using the user's user attribute information 51, medical examination information 52, and processing definition information 58, the analysis unit 16 performs each processing including notice information extraction such as tendency analysis and disease risk determination, and action extraction. The analysis unit 16 performs processing for various types of tendency analysis of the user's health data 53, examination result data 54, and calendar input information 55 and stores the result information in the analysis information 56. The analysis unit 16 performs action extraction processing from the data such as the health state according to the analysis information 56 of the user and the actions registered in the calendar input information 55 and stores the result information in the analysis information 56. The analysis unit 16 performs disease risk determination processing using the health state according to the analysis information 56 of the user and a combination of the elements of the above health data and stores the result information in the analysis information 56.

Based on the above analysis information 56, the message output unit 17 performs processing for outputting information, which includes a message appropriate for the health state of each user, on the screen of the terminal 2 of the user and manages the information as the output message information 57. The output message information 57 includes definition information on each message and management of history information in time series.

The auxiliary unit 18 performs processing corresponding to other functions of the present service in cooperation with the application 20 and manages the information therefor in the DB 50.

[Functions and Data]

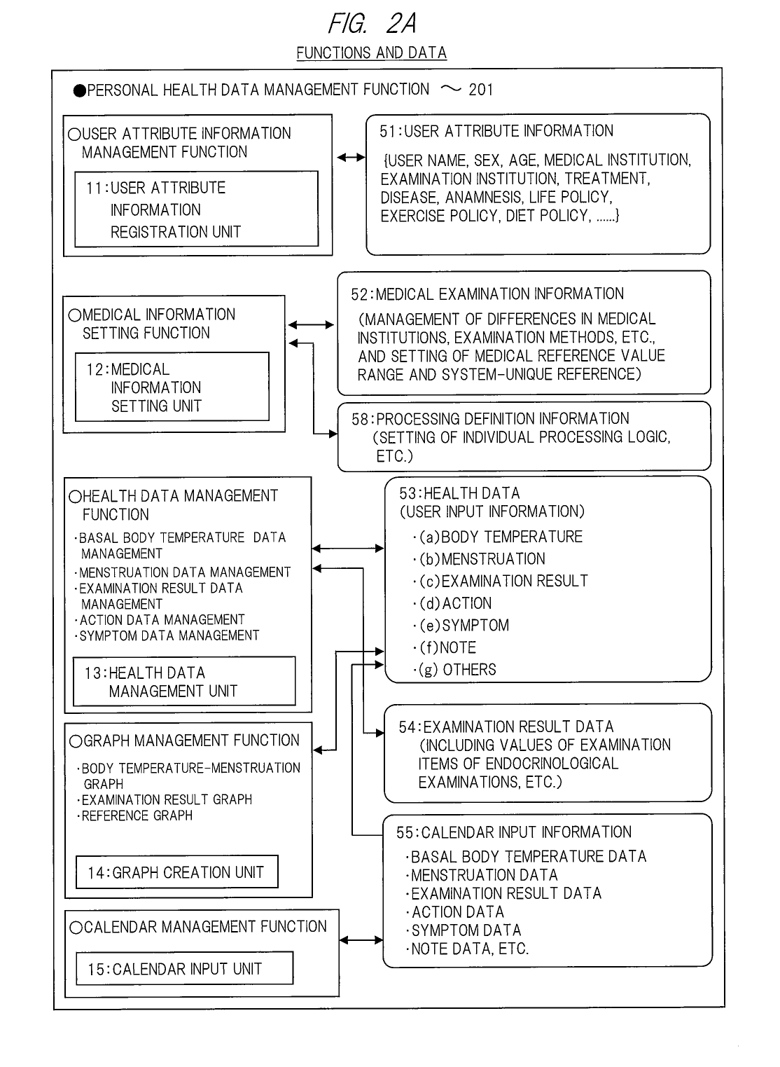

FIGS. 2A and 2B show service and corresponding functions provided and outlines of data and information managed by the health care system according to the first embodiment. The health care system according to the first embodiment includes, as the main functions thereof, (1) a personal health data management function 201, (2) an analysis and message output function 202, and (3) other functions 203.

(1) In FIG. 2A, the personal health data management function 201 includes a user attribute information management function, a medical information setting function, a health data management function, a graph management function, a calendar management function, and the like, and manages the user attribute information 51, the medical examination information 52, the processing definition information 58, the health data 53, the examination result data 54, the calendar input information 55, and the like. The personal health data management function 201 includes a function of registering and managing various data including body temperature and the like relating to the health state of each individual user. The personal health data management function registers user input information such as body temperature input daily at any time by the user through the screen of the application 20 of the terminal 2, in the DB 50 as the health data 53.

(1-1) The user attribute information management function is realized by using the user attribute information registration unit 11 and is a function including registration and management of the user attribute information 51 on each user. The user attribute information 51 includes, as items, user name, sex, age, medical institution and examination institution used, states of treatment, disease, and anamnesis, life policy, exercise policy, and diet policy.

(1-2) The medical information setting function is realized by using the medical information setting unit 12 and is a function of setting and managing the medical examination information 52 and the processing definition information 58 based on operation of the administrator.

In the medical examination information 52, information on each of a plurality of medical institutions and examination institutions is set and managed. The present system uses the medical examination information 52 to manage differences in medical institutions, examination methods, and the like which each user uses, and provides analysis and the like in consideration of the differences. The medical examination information 52 includes settings and management of a medical reference value range for each medical institution and examination institution and of unique reference information for control in the present system.

In the processing definition information 58, definition information on individual processing logic used for various analyses, checks, and the like by the analysis functions is set. The processing definition information 58 includes management of reference information to be applied based on the medical examination information 52.

(1-3) The health data management function is realized by using the health data management unit 13, and includes a function of recording and centrally managing data of each element for each individual user. The health data management function includes functions of basal body temperature data management, menstruation data management, examination result data management, action data management, symptom data management, and the like.

The health data 53, which is the user input information, includes, as elements, (a) basal body temperature, (b) menstruation, (c) examination result, (d) action, (e) symptom, (f) note, and (g) others. The examination result of (c) includes values of a plurality of types of endocrinological examination items, and the like. The action of (d) includes exercise therapy, diet therapy, music therapy, and the like. The symptom of (e) includes stress. The note of (f) includes an arbitrary text which expresses a feeling, a memo, and the like. Information on others of (g) includes information on treatment, examination, prescription, and the like as relevant information on the health state. Information on the prescription (also called medication) includes information on prescription of a medicine by a medical institution, taking medicine by a user, and the history thereof. A specific example is “Apr. 1, 2012: Medicine A” or the like. Note that, of the health data 53, the examination results are separated as the examination result data 54, but these are information with the same contents. In the health data 53, data is managed by each element such as body temperature.

(1-4) The graph management function is realized by using the graph creation unit 14 and manages information on graph data including a body temperature-menstruation graph, an examination result graph, and the like, which represent the health state of the user. The graph management function sets and manages information on a reference graph, which will be described later, separately from the graph of each user.

(1-5) The calendar management function manages a screen including a calendar for registering and displaying the user input information, and the calendar input information 55. The calendar management function displays a calendar on the screen of the application 20 of the terminal 2 of the user and controls registration and display of the basal body temperature, the menstruation, the examination result, the action, the symptom, the note, the treatment, the medication, and other information on the calendar date. The user input information is recorded and centrally managed in time series in the calendar format. With the calendar, it is also possible to review the information recorded in the past and plan and schedule future actions and the like.

(2) In FIG. 2B, the analysis and message output function 202 includes an analysis function and a message output function. The analysis function includes a notice information extraction function and an action extraction function, performs analysis processing based on the processing definition information 58, and manages the analysis information 56. The notice information extraction function includes a tendency analysis function and a disease risk warning function. The message output function is realized by using the message output unit 17 and manages the output message information 57. The analysis and message output function 202 performs advanced analysis of the health state of each user by using each data by the personal health data management function 201 of the above (1), that is, the health data 53, the user attribute information 51, and the like of each individual user. Then, the analysis and message output function 202 outputs an advanced message appropriate for the health state of each user based on the analysis results.

(2-1) The tendency analysis function includes functions of (2A) tendency analysis of body temperature and menstruation, (2B) tendency analysis of examination results, (2C) tendency analysis of actions, and (2D) tendency analysis of symptoms. The processing for the tendency analyses determines absolute good or bad and tendencies such as relative improvement, deterioration, and the like in the values and states of the body temperature and the like, of the health data of the user, based on predetermined values. The tendency includes time series variations in values.

(2A) The function of the tendency analysis of the body temperature and the menstruation analyzes the health state including the tendency of the body temperature and the menstruation of the user by using the values and the graphs in the health data 53 and the like of each user. This function includes determination and calculation of values of predetermined items such as a temperature difference and a menstrual cycle, which will be described later.

(2B) The function of the tendency analysis of the examination results analyzes the health state including the tendency of the examination results of the user by using the values and the graphs of the examination result data 54 and the like of each user. This function includes determination and calculation relating to the examination results.

The functions of the tendency analyses of the body temperature, the menstruation, the examination results, and the symptoms perform the analyses by using the health data 53 of each user. The tendency includes, for example, a change (amount, frequency, and continuity) in each health data during a certain period in the past.

(2-2) The action extraction function uses action data of each user to extract life habit information, which includes past actions assumed to be relevant to and influencing the current health state of the user such as body temperature, menstruation, examination results, and symptoms, and presents the information to the user. For the health state of the user, the results of the tendency analyses are used. The action extraction function may extract not only life habits including actions, but also information on relevant symptoms and the like.

(2-3) The disease risk warning function estimates and checks the health state of the user, including the disease and the like, by comprehensive analysis using the above elements such as the user's body temperature, the menstruation, the examination result, the action, the symptom, and the note in combination. Then, the disease risk warning function outputs a message appropriate for the results by using the message output function. Depending on the results, the disease risk warning function outputs a message warning of the possibility and the risk of the disease. The check targets include various women's diseases and the like. In other words, warning is an alert which suggests the possibility and calls attention.

The outline of the output message information 57 by the above function of (2) includes the following. The output message information 57 includes general medical knowledge, the latest information, tendency analysis result information, extracted actions, action tendency, life advice, disease risk warning information based on the check results, consultation recommendation for treatment, examination, hospitals, and the like, for example. The tendency analysis result information includes information conveying the values of the health state of the user, whether the values are good or bad, and tendencies such as improvement and deterioration of the values. The output message information 57 is helpful information based on the specific analyses conducted by the present system and provided to each user.

(3) The other functions 203 are auxiliary functions and are realized by using the auxiliary unit 18. The other functions 203 include an input assistance function, a graph interpolation function, a graph matching function, a relevant information search function, and the like. The input assistance function is a function of assisting the user to input data and includes a medical device cooperation function and a voice input function. The graph interpolation function includes a function of creating a graph of the user by interpolating values. The graph matching function includes a function of comparing the graph of the user with the reference graph. The relevant information search function includes a function of automatically searching for and presenting relevant information on the health state of each user and the output message for each user.

[Data Input]

Specific examples of inputting and registering data such as body temperature and examination results in the system in FIG. 1 are shown below. First, an input of body temperature and menstruation data is as follows. The user measures the basal body temperature daily with the medical device 3 such as the thermometer. On the screen of the application 20 of the terminal 2, the user inputs the basal body temperature and, in the case of having menstruation, inputs information such as a menstruation date. The user may manually input values of a paper basal body temperature table on the screen of the application 20 or may import the values as data by scanning or photographing the paper. In the case of manual input, the user can display an input field of the body temperature on the screen of the application 20 and select and input a date and a value. Alternatively, the user can display a graph field of the body temperature on the screen of the application 20 and input a value by plotting at an appropriate date.

Alternatively, the user may input the body temperature data and the like from the medical device 3 by communication through the body temperature-menstruation data input unit 21 of the terminal 2. With the processing of the application 20 of the terminal 2 and the auxiliary unit 18 of the service unit 10, the medical device cooperation function of the input assistance function is realized. For example, when the user holds or connects the medical device 3, which is the thermometer, against or to an interface unit of the body temperature-menstruation data input unit 21 of the terminal 2, the body temperature data is transferred from the medical device 3 and input. The application 20 of the terminal 2 saves the input data of the body temperature, the menstruation, and the like in the terminal 2 and transmits the data to the server 1 to be registered.

By the application 20 and the service unit 10, the input data such as body temperature is converted as appropriate into data in a predetermined format handled by the present system. Moreover, when the medical device 3 keeps the body temperature data in time series or in a graph format or has information such as menstruation, height, weight, and body mass index (BMI) besides the body temperature all together, these data may be collectively input into the application 20 of the terminal 2. In the medical device 3, an actions, a symptom, a note, and other information may be input.

Next, an input of the examination result data is as follows. The input will be described together with usage examples of premised medical institutions and examination institutions. For example, a user who undergoes treatment and examination of infertility goes to the department of obstetrics and gynecology or the like at a hospital. A doctor examines the user who is a patient, conducts examination, orders a prescription, diagnoses the medical condition, and performs treatment such as medical activities as necessary. The treatment includes the timing method, treatment of diseases causing infertility, artificial insemination, and the like.

An examiner, who belongs to an examination company which is an examination institution received an order of the examination, an examination department in the hospital, or the like, conducts the examination ordered. The examination institution, for example, as a blood test, measures values of female hormones and the like contained in the blood of the user, which is the specimen, by using an examination device and records the examination result data of the user in the terminal 4 and the like.

The user inputs the examination result data by the terminal 2 by using the examination result paper or the examination result data provided by the examination institution or the like. The application 20 of the terminal 2 displays a screen including an input field of the examination result data. On the screen, the user can input the date of the examination, the medical institution and examination institution used, the examination items, the values, and the like. Moreover, in particular, the examination result data transferred from the terminal 4 can be input collectively by the terminal 2 of the user. The terminal 2 saves, in the terminal 2, the examination result data input through the examination result data input unit 22 and transmits the data to the server 1 to be registered.

Note that, among the user, the medical institution, the examination institution, and the provider, the data and information on the user may be provided from the terminal 4 of the medical institution and the like to the terminal 2 of the user or to the server 1 based mutual agreements. Subjects to be provided are information on the treatment, the medical condition, the clinical examination, and the examination which is recorded in the clinical record of the medical institution, the data of the body temperature and the menstruation measured at the medical institution, the examination result data by the examination institution, and the like. In this case, time and effort of the user can be reduced for the data registration.

Moreover, a paper or data of the examination results may be transmitted from the user, the examination institution or the like to the provider via mail or the communication network 9, and the provider may make data from the paper or the data as the examination result data 54. Furthermore, in the case of an examination conducted by the user himself or herself using a test drug or the like, the value of the measurement by the user may be input by the terminal 2 and registered as the examination result data 54.

The case of using the voice input function of the input assistance function is as follows. The terminal 2, the application 20, or the service unit 10 is provided with a known voice recognition function as an element constituting the voice input function. To input data of the body temperature and the like with the application 20 of the terminal 2, the user selects use of the voice input function and inputs, for example, the value of the body temperature by voice. For example, the voice recognition function of the application 20 recognizes the input voice of the user, converts the voice into voice data, analyzes the voice data, and extracts information such as the value of the body temperature. The application 20 transmits the voice data or the extracted information to the server 1, and the server 1 registers the body temperature data from the voice data or the extracted information. The same applies to the case where the server 1 performs the analysis.

[Processing]

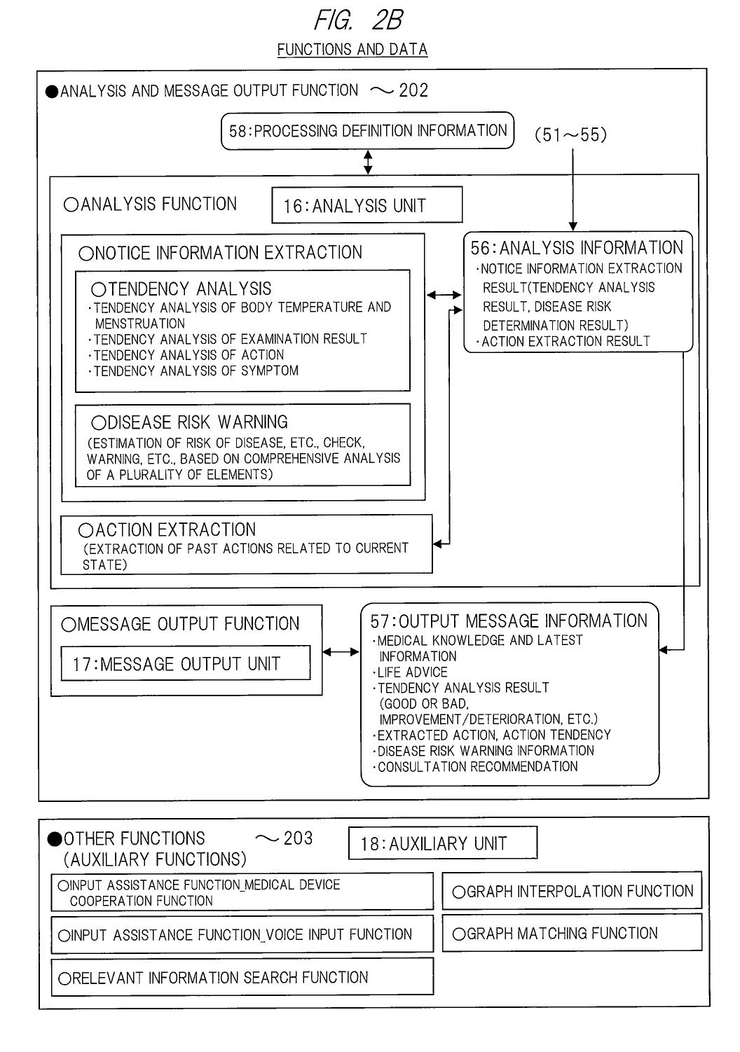

FIG. 3 shows a flow of main processing by the application 20 and the server 1. Reference character S1 and the like indicate processing steps.

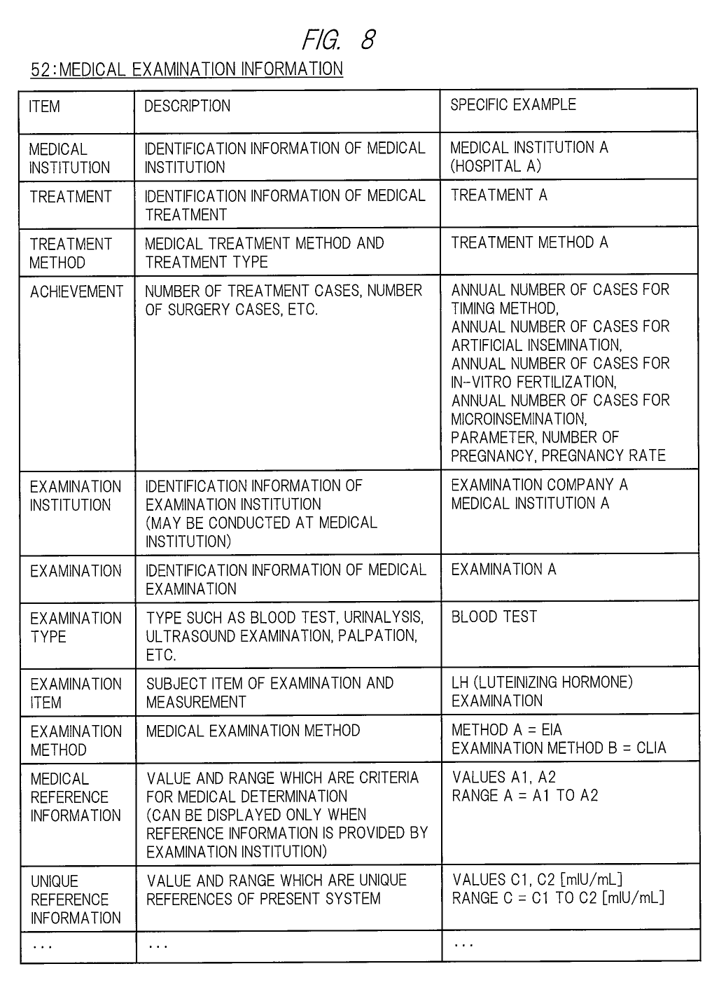

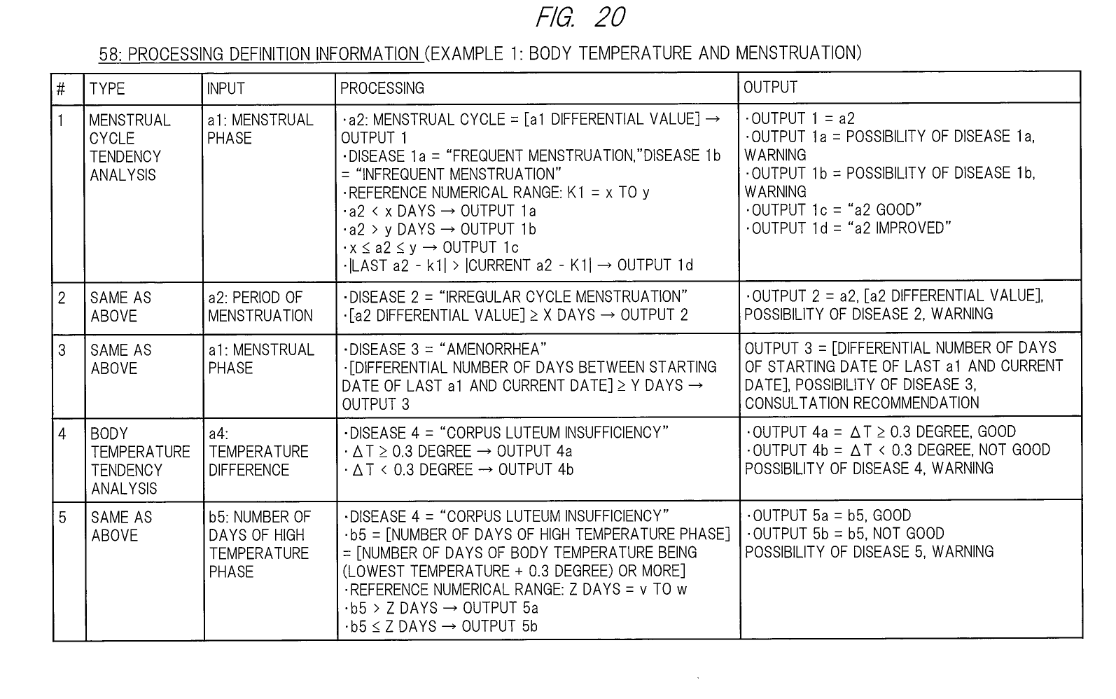

(S1) In the server 1, the administrator and the medical information setting unit 12 sets in advance the medical examination information 52 (described later in FIG. 8) and the processing definition information 58 (described later in FIG. 20 and the like), which are the management information of the present system. The setting contents of the management information are updated as necessary according to the addition, modification, and the like of the information on the medical care and the examination.

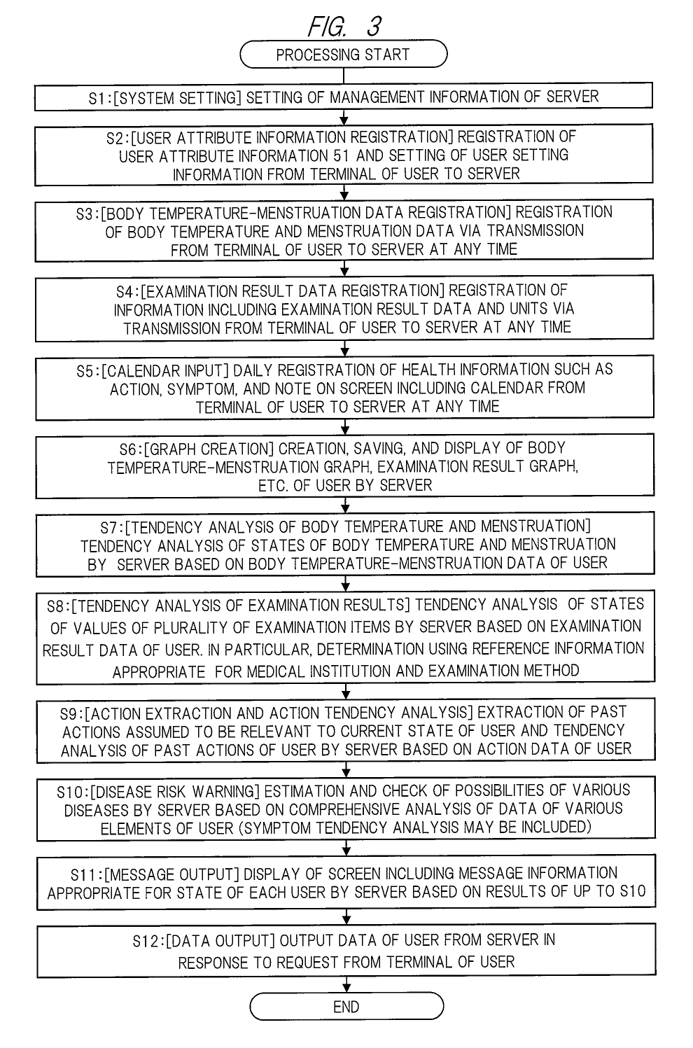

(S2) Based on operation of the user, the application 20 of the terminal 2 accesses the service unit 10 of the server 1, and a screen of the service is provided to the terminal 2. The screen is a screen for registration of the user attribute information provided at the start of service use or as necessary, for example. The screen includes an input field of each attribute item of the user, and the user can register the information by choices, values, text, and the like in each item. The user attribute information registration unit 11 registers the information input on the screen in the user attribute information 51 (described later in FIG. 4). The user can update the contents of the user attribute information 51 at any time when the user has undergone the treatment, the examination, and the like. Moreover, the user can set the user setting information for himself or herself on the screen of the service as appropriate.

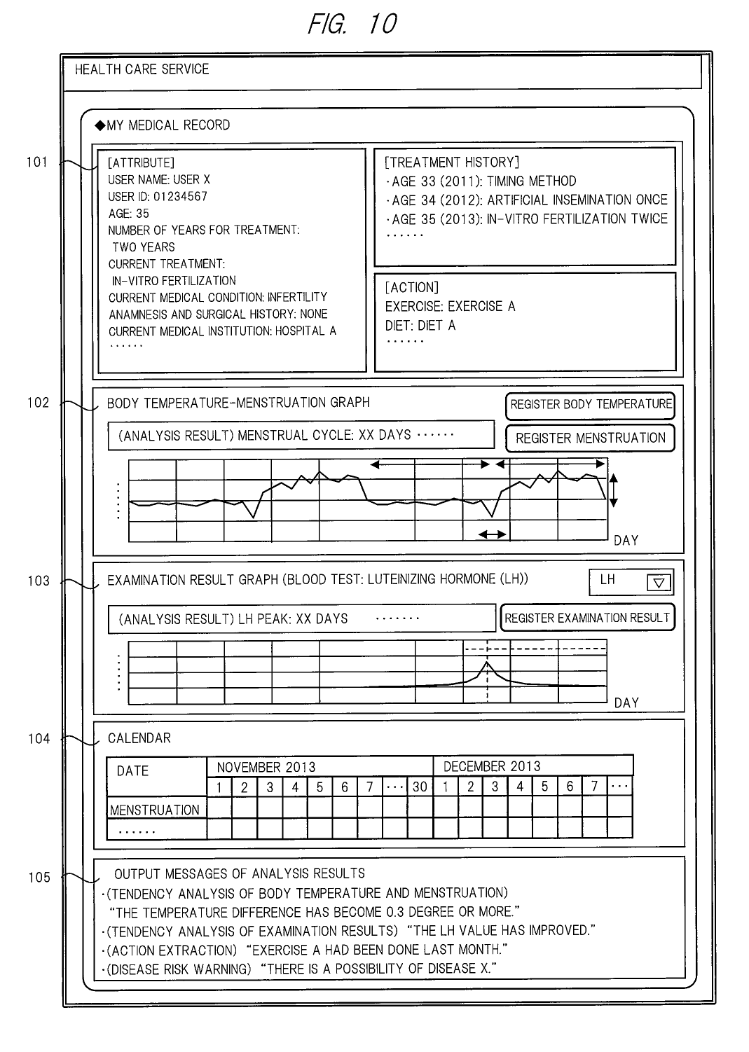

(S3) At any time, the application 20 of the terminal 2 of the user accesses the service unit 10 of the server 1, and the health data management unit 13 provides a screen (described later in FIG. 10 and the like) including input fields of the body temperature and the menstruation data. On the screen, the user inputs information on his or her body temperature and menstruation based on, for example, the body temperature data from the medical device 3. The application 20 of the terminal 2 transmits the body temperature and the menstruation data of the user to the server 1, and the health data management unit 13 registers the data as the health data 53.

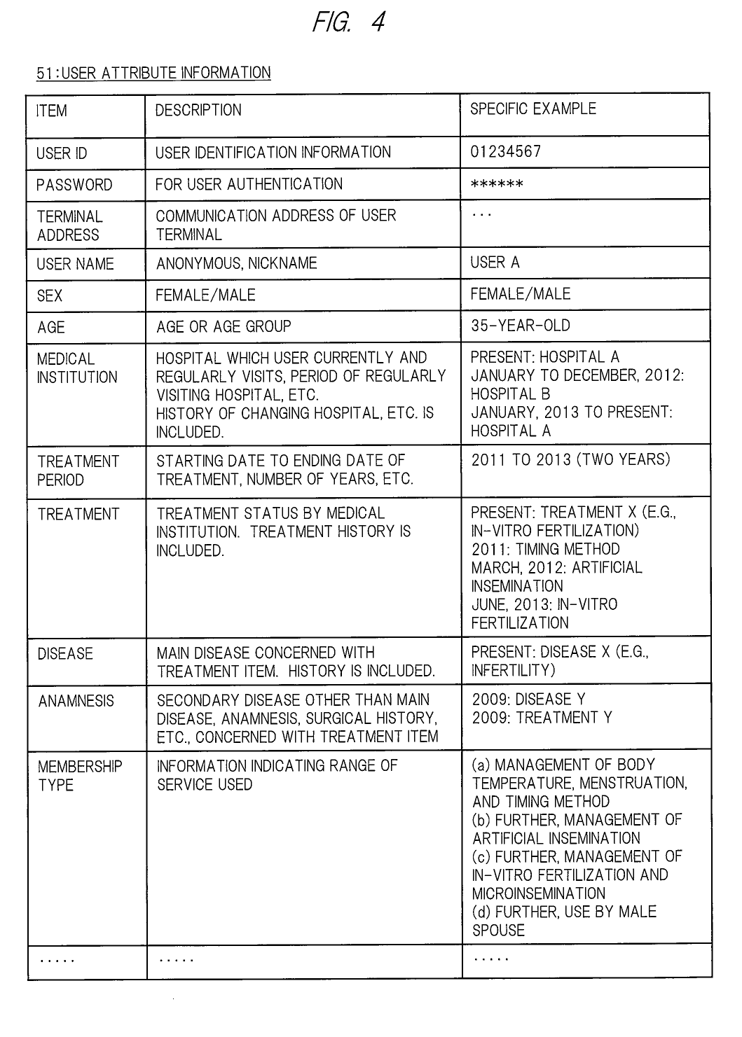

(S4) Likewise, at any time, the terminal 2 of the user accesses the server 1, and the health data management unit 13 provides a screen (FIG. 10 and the like) including the input field of the examination result data. On the screen, the user inputs the examination result data of the user based on, for example, the examination result data from the terminal 4 of the examination institution. The application 20 of the terminal 2 transmits information including the examination result data of the user and the units thereof to the server 1, and the health data management unit 13 registers the information as the examination result data 54 (described later in FIG. 5). The units are, for example, [ng/mL] or [pM] for an AMH item described later.

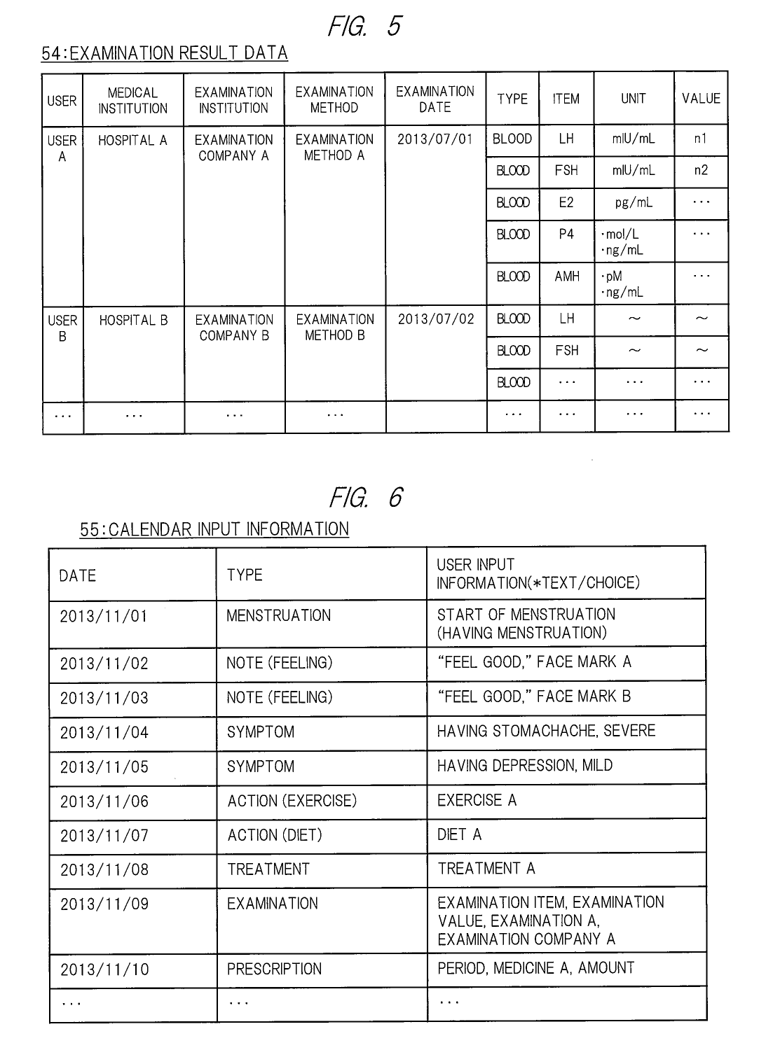

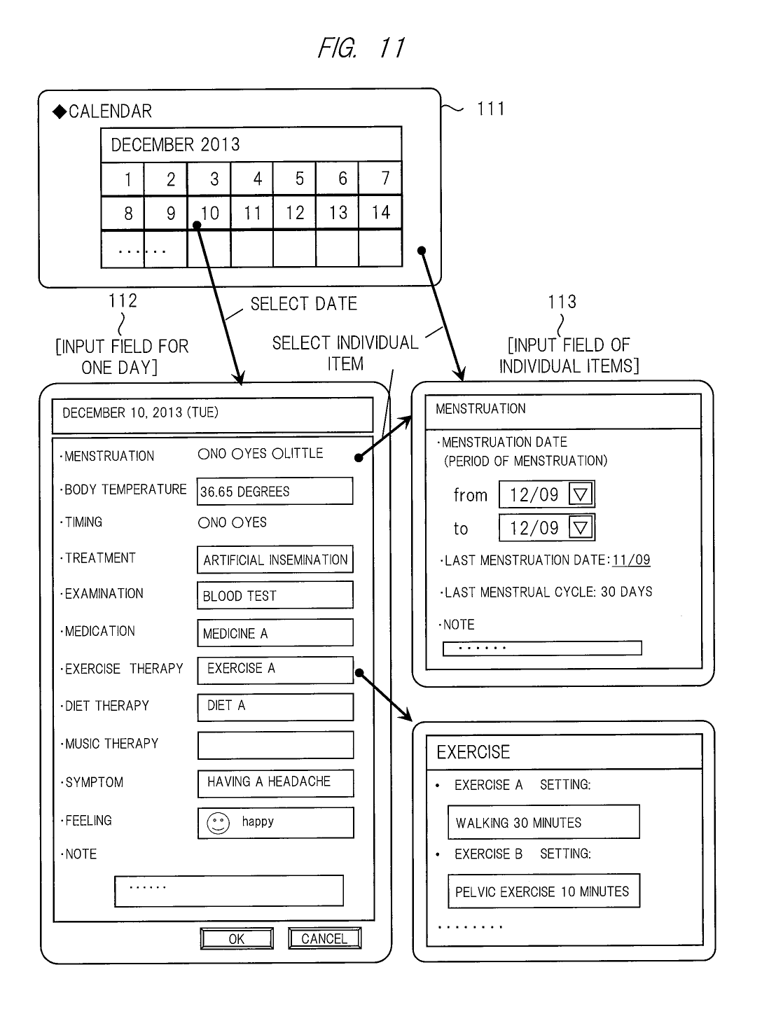

(S5) At any time, the application 20 of the terminal 2 of the user accesses the service unit 10 of the server 1, and the calendar input unit 15 provides a screen including a calendar (described later in FIG. 11 and the like). The user can input information on various elements such as the user's body temperature, menstruation, e examination result, action, symptom, and note on the date in the calendar. These pieces of information can be entered by text or by selecting a predetermined choice, a mark, and the like. The calendar input unit 15 registers various types of information input by the user in the calendar input information 55 (described later in FIG. 6). As described above in the steps S3 to S5, the user can input and register various kinds of information such as the health data at any time on a daily basis on the screen of the terminal 2 of the user.

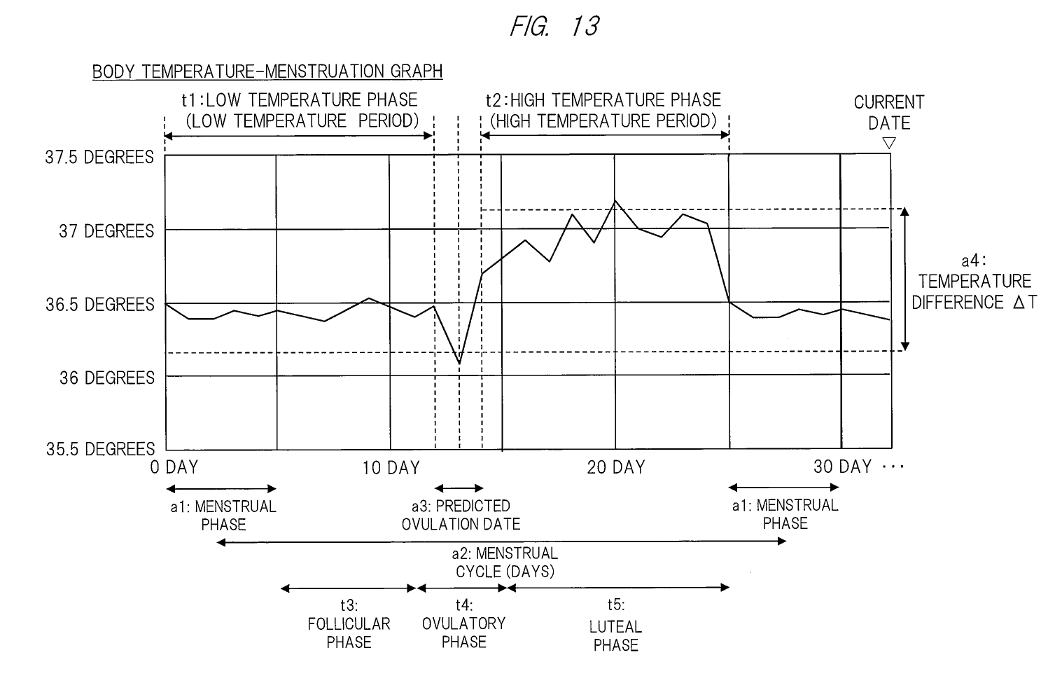

(S6) The graph creation unit 14 of the server 1 uses the health data 53 by the step S3 to create or update a body temperature-menstruation graph (described later in FIG. 13 and the like) for each user and saves the graph as a part of the health data 53. The body temperature-menstruation graph is a graph based on the time series values of the basal body temperature and is a graph in which information such as a menstruation date and a menstrual cycle is overlapped. The graph creation unit 14 provides the terminal 2 of the user with a screen including the created body temperature-menstruation graph and the relevant information.

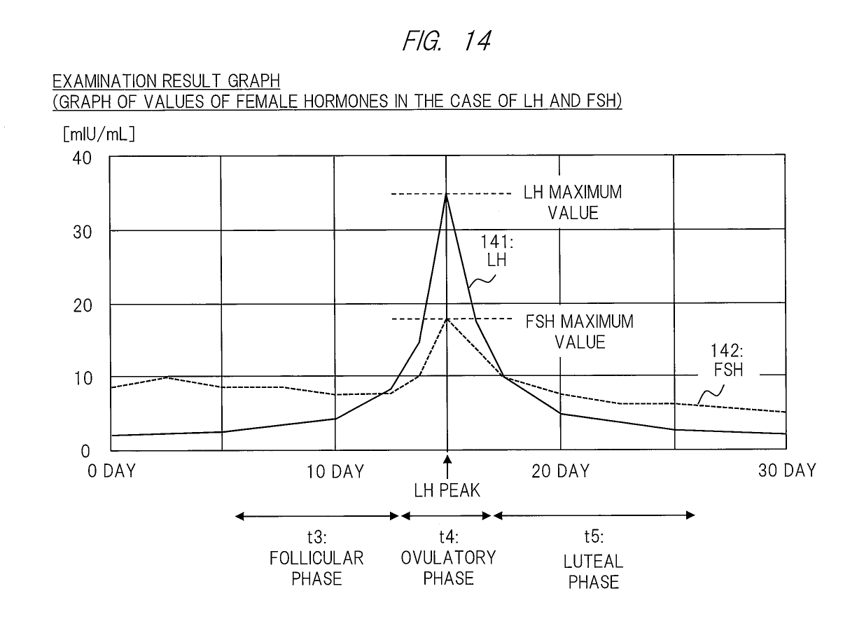

Moreover, the graph creation unit 14 uses the examination result data 54 by the step S4 to create or update an examination result graph (described later in FIG. 14 and the like) for each user and saves the graph as a part of the examination result data 54. The examination result graph is a graph of the time series values relating to a plurality of types of examination items, for example, various types of female hormones by blood test. The graph creation unit 14 provides the terminal 2 of the user with a screen including the created examination result graph and the relevant information.

(S7) The analysis unit 16 of the server 1 uses the health data including the above registered health data 53 of the user to perform tendency analysis processing for the body temperature and the menstruation of each user and stores the results thereof in the analysis information 56. Based on the processing definition information 58, the analysis unit 16 uses the user's user attribute information 51, body temperature-menstruation graph, calendar input information 55, and the like to determine good or bad and the state of tendency such as improvement or deterioration, of the body temperature and the menstruation of the user. The analysis unit calculates and records values of the user's temperature difference, menstrual cycle, predicted ovulation date, and the like and calculates amounts of time series changes in these items to determine the tendency. Moreover, the analysis unit 16 compares the user's values with reference numerical ranges to determine the state.

(S8) The analysis unit 16 of the server 1 uses the health data including the above registered examination result data 54 of the user to perform the tendency analysis processing for the examination results of each user and stores the results in the analysis information 56. Based on the processing definition information 58, the analysis unit 16 determines good or bad and states of tendencies such as improvement or deterioration, of the values of a plurality of examination items, for example, the values of a plurality of kinds of female hormones. The analysis unit 16 calculates amounts of time series changes in the values of a plurality of examination items of the user and determines the tendency. Moreover, the analysis unit 16 compares the user's values with reference numerical ranges to determine the state. In the steps S7 and S8, the analysis unit 16 refers to the medical examination information 52, applies the reference information appropriate for the differences in the medical institutions, examination methods, and the like used by the user and performs the above tendency analyses.

(S9) The analysis unit 16 uses the health data including the above registered action data to perform processing for the action extraction and the action tendency analysis for each user and stores the results thereof in the analysis information 56. In the action tendency analysis processing, the analysis unit 16 determines a tendency of the actions of the user in the past period. For example, the analysis unit 16 calculates an amount, frequency, continuity, and the like of each action type such as diet or exercise in values and determines time series changes thereof.

In the action extraction processing, the analysis unit 16 extracts information on the past actions of the user, which are assumed to be relevant to or influencing the user's current health state detected by the tendency analyses in the steps S7 and S8. The analysis unit 16 determines the action to be extracted by using the user's user attribute information 51, the body temperature-menstruation graph, the examination result graph, the registration information such as the action, the symptom, and the note of the calendar input information 55, and the analysis information 56 thereof.

The action extraction processing is specific processing for mildly assuming past actions and the like which are likely to be related to the current health state of the user and is intended to make the extracted information useful for the user as the helpful information. In the step S9, not only processing for the analysis and the extraction of the action data but also analysis and extraction of relevant symptom data and the like in time series may be performed likewise. To analyze and extract the symptoms, the analysis unit 16 uses the symptom data of the user to calculate an increase or decrease in the number of various symptoms, calculates an amount of variation thereof in time series and determines the state of improvement or deterioration of the symptoms based on the comparison between the amount of variation and the predetermined values.

(S10) The analysis unit 16 uses various types of information such as the above registered health data in combination to perform processing for comprehensive disease risk warning and stores the results thereof in the analysis information 56. In the disease risk warning processing, the analysis unit 16 uses each element such as body temperature, menstruation, examination result, action, symptom, and note in combination to mildly estimate the possibilities of various women's diseases based on the processing definition information 58. In the step S10, the analysis unit 16 may also perform symptom tendency analysis processing together to confirm the disease risk.

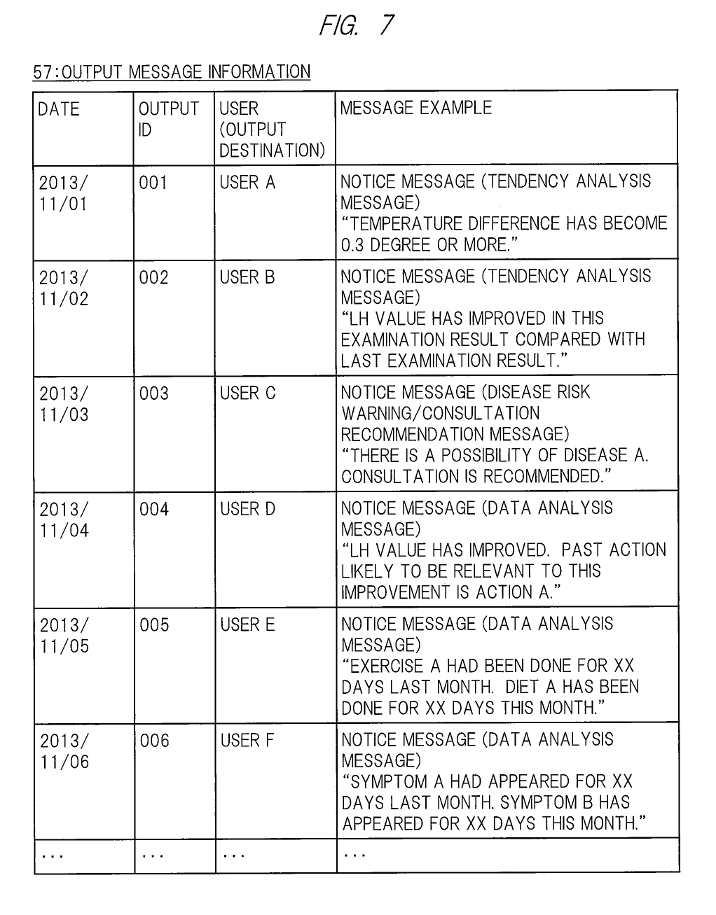

(S11) The analysis unit 16 of the server 1 determines an output message appropriate for the health state of each user based on the analysis information 56 including the results of the above steps S7 to S10. The message output unit 17 displays information including the message on the screen of the terminal 2 of the user. The output message may be displayed in a dedicated field or a corresponding graph field on the screen. The message output unit 17 stores the output message as a history in the output message information 57 (described later in FIG. 7). The timing of outputting the message may be at the time of receiving a request from the user, at the time of analyzing the data of the user, or at the periodic time based on the user setting such as every day, every predetermined number of days, or the like.

As described above, at any time on the screen (FIG. 10 and the like) of the terminal 2 of the user, the user can browse the information such as the user's registered body temperature, menstruation, examination result, action, symptom, and note and can also browse various graphs and output message information on the analysis results. On the screen, the user can browse selected individual information, browse a list of a plurality of types of information or browse a plurality of types of information in parallel, browse information on a daily basis, browse information in a designated period of the past, and the like.

(S12) In response to a request from the application 20 of the terminal 2 of the user to output desired data by the user, the server 1 reads out the corresponding data saved in the DB 50 and transmits the data to the terminal 2. In the DB 50, each data of each user is organized and accumulated. The application 20 of the terminal 2 of the user saves the data received from the server 1 in a memory and performs screen display and printing. The data which can be output includes the user's user attribute information 51, each graph, calendar input information 55, output messages for the analysis results, and the like. The output data can be a file of history information and list information in a unit of a designated period such as past one month. The user can utilize the output data for confirmation and submission upon clinical examination at medical institution, and the like. Moreover, to output the examination result data in the step S12, the server 1 performs unit conversion on the values of the examination items and provides the data after the unit conversion.

[User Attribute Information]

FIG. 4 shows a configuration example of main data items of the user attribute information 51. The user attribute information 51 constitutes the user information for the present service and stores various attribute information on the health state of the user, that is, attribute values, in addition to the basic information on the user. The user attribute information 51 in FIG. 4 includes, as items, user ID, password, terminal address, user name, sex, age, medical institution, treatment period, treatment, disease, anamnesis, membership type, and the like.

The user ID, the password, the terminal address, and the like are basic information on the user for service control. The terminal address is an IP address, a telephone number, an e-mail address, and the like. The basic information may include a mailing address and the like. The “user name” item is anonymous or a nickname set by the user. The “age” item is an age or an age group.

The “medical institution” item includes identification information on a medical institution such as a hospital which the user currently uses or regularly visits, and an examination institution. The “medical institution” includes management of history of changing a hospital and the like, and includes, for example, a hospital name, a period of regularly visiting a hospital, and the like. Specific examples are “Present: Hospital A,” “January to December, 2012: Hospital B, January, 2013 to Present: Hospital A,” and the like. Note that “Hospital A” and the like indicate abstracted identification names for explanation.

The “treatment period” item indicates a period from the starting date of the treatment to present or to the ending date, the number of years for the treatment, and the like. The treatment referred to in this item indicates overall approach, and individual treatment is managed in the following items.

The “treatment” item is information which indicates the treatment status by the medical institution, and a name and identification information are registered here. The “treatment” includes the practice of therapy by the user. The “treatment” includes management of the history of the treatment. The “treatment” includes management of information on the course of treatment, the states such as start and end, and the details of the treatment. Specific examples are “Present: Treatment X=In-Vitro Fertilization,” “2011: Timing Method,” “2012: Artificial Insemination,” “2013: In-Vitro Fertilization,” and the like.

The “disease” item is information which indicates the current major disease or medical condition of the user and which is concerned with the above “treatment” item, and the name of and identification information on the disease are registered here. The “disease” includes management of the history. The “disease” includes management of information on the course of the disease, the states of the start and end thereof, and the details of the disease. The “disease” includes management of the state of the presence of the possibility of the disease and the state of health. The “disease” includes management of the state relating to pregnancy, infertility, and childbirth (e.g., success or failure of pregnancy). Specific examples are “Present: Disease X=Infertility” and the like.

The “anamnesis” item stores outline information such as the user's relevant chronic disease, anamnesis, and surgical history other than the values of the above “disease” and “treatment” items. That is, the “anamnesis” item manages information on the secondary disease and treatment. The “anamnesis” includes diseases and treatment in other medical fields, not limited to the fields of the obstetrics and gynecology. Specific examples are “2009: Disease Y, 2009: Treatment Y” and the like. Note that the “anamnesis” item may be integrated into the “disease” item and the like to be managed.

The registrations of the above items of “treatment,” “disease,” “anamnesis,” and the like are not limited to text input by the user, and the registrations are also possible by selecting a choice of treatment and illness preset in the present system. The names of treatment and diseases, including those which are not unified, are set in the present system.

The present system may provide different services and functions depending on the status of the membership type and the like of each user. The server 1 manages, for example, information for associating the user ID and the like with the membership type, services, and functions. In the “membership type” item, information on the membership type of the user is registered. The membership type is associated with a range of the services and functions used. For example, the membership types are classified into the following (a) to (d). (a) is a membership type which uses relevant services and functions, including management of body temperature, menstruation, and the timing method. (b) further includes management of artificial insemination in addition to (a). (c) further includes management of in-vitro fertilization and microinsemination in addition to (b). (d) further indicates use also by a male spouse. For example, a first user uses (a), a second user uses (b), a third user uses (c), and a fourth user uses (d).

The user attribute information 51 may provide height, weight, and the like as other items and may also provide items such as insurance, family, occupation, region, drinking alcohol, and smoking. The analysis unit 16 uses the information in each item of the user attribute information 51 upon the analyses. The user may input the information given by the medical institution or the like into the user attribute information 51 or may input information based on self-judgment.

[Examination Result Data]

FIG. 5 shows an example of the examination result data 54 of each user. The health data 53 and the examination result data 54 are managed in association with the user attribute information 51 and the medical examination information 52. The table of the examination result data 54 in FIG. 5 includes, as items, user, medical institution, examination institution, examination method, examination date (including time, in some cases), type, item, unit, and value. The “user” is a user ID or a user name. The “medical institution” indicates a hospital and the like used by the user. The “examination institution” indicates an examination company and the like used by the user. When the medical institution and the examination institution are the same, the value can be omitted. The “examination method” is information which indicates an examination method employed for the examination by the examination institution. The “examination date” is the date on which the examination was performed. The “type” is a type of examination such as blood test, ultrasound examination, and semen examination. The “item” is an examination item or an examination subject and is, for example, a specific female hormone. As a plurality of types of endocrinological examinations and the like, an LH and an FSH described later are the examination subjects, for example. The “unit” is a unit of the value of the examination item. Note that, as for the unit, two or more units may be used, in some cases. The “value” is a value of the examination item.

For example, the first row shows that the user A is concerned with treatment at the hospital A and has undergone an examination with the examination method A by the examination company A, a blood test was performed on, for example, July 1, and the values of a plurality of types of endocrinological examinations were LH=n1, FSH=n2, and the like. Note that, in an unillustrated example of the health data 53, for example, information is similarly managed with the items such as user, date, body temperature value, and presence or absence of menstruation.

[Calendar Input Information]

FIG. 6 shows a management example of the calendar input information 55. The table of the calendar input information 55 in FIG. 6 includes date (including time, in some cases), type, and user input information as items. The date is the date on which the user input information is registered, corresponding to the date of the calendar. The type indicates a rough type of the user input information. In the example in FIG. 6, the type indicates menstruation, note, symptom, action, treatment, examination, prescription, and the like. The user input information indicates the text input by the user, selected choice, identification information of marks, and the like.

In the example in FIG. 6, November 1 is registered as a menstruation date, that is, presence of menstruation. On November 2, the text “feeling good” of the note and the face mark A representing a feeling and the like are registered. On November 4, the symptom is registered where there is a stomachache and the degree thereof is severe. On November 6, exercise A is registered for action, specifically exercise. On November 8, treatment A is registered for treatment. On November 9, examination items, examination values, examination A, and examination company A are registered for the examination. On November 10, a period, a medicine A, and an amount are registered for the prescription or the medication.

The symptoms, the actions, and the like of the user can be input by choices, marks, and the like prepared and set in advance in the present system and can be also input with free text. The present system may set common actions, common symptoms, and the like as the choices. For example, to register an emotion, the text of the note will be “stressed out,” “disappointed,” or the like.

[Output Message Information]

FIG. 7 shows a configuration example of the output message information 57. The output message information 57 is managed in time series, including the information planned to be output and the history of the information output in the past. The table of the output message information 57 in FIG. 7 includes date (including time, in some cases), output ID, user, and message example as items. The “date” indicates the date on which the message is output or the date on which the message was output. The “output ID” is identification information on the output. The “user” indicates the user ID and the like of the output destination of the message. The “message example” is the text of the contents of the output message and may be the identification information thereof. Since the past output messages are also managed by history, the user can reconfirm, on the screen, the past output messages, for example, the contents of the warnings and the like on the past dates. In the output message information 57, an item of message type (e.g., “tendency analysis,” “warning,” or the like) may be managed.

In the example in FIG. 7, the output ID=001 shows “the temperature difference has become 0.3 degree or more” as a tendency analysis message which is a notice message for the user A on November 1. Another example is “the temperature difference has become less than 0.3 degree.” Another example is “the menstrual cycle has extended one day from 30 days to 31 days” or the like. These are examples of the tendency analyses of the body temperature and the menstruation and include comments on the states of the body temperature and the menstruation and the tendencies such as variations.

The output ID 002 shows an output example of a tendency analysis message which is a notice message, “the LH value has improved in this examination result compared with the last examination result.” Another example is “the FSH value has deteriorated compared with the last menstrual cycle” or the like. These are examples of tendency analyses of the examination results and include comments on the states and the tendencies.

The output ID 003 shows an output example of a disease risk warning and consultation recommendation message as a notice message, “there is a possibility of disease A. Consultation is recommended.” This is an example of a warning (alert) of the possibility of disease and consultation recommendation by the disease risk warning processing.

The output ID 004 shows an output example of a data analysis message as a notice message, “the LH value has improved. The past action likely to be relevant to this improvement is action A.” This is an example of action extraction based on the tendency analysis results. Another example is “there is a possibility that the LH value has improved due to the influence of action A” or the like.

The output ID 005 shows an output example of a data analysis message as a notice message, “exercise A has been done for XX days last month. Diet A has been done for XX days this month.” This is an example of the action tendency analysis and the action extraction.

The output ID 006 shows an output example of a data analysis message as a notice message, “symptom A had appeared for XX days last month. Symptom B has appeared for XX days this month.” This is an example of the symptom tendency analysis and the symptom extraction.

As another output example, an example of explanation and advice based on medical knowledge is “there is a symptom called premenstrual syndrome (PMS) accompanied by somatic symptoms and physical symptoms in a period from two weeks before the menstruation to right before the menstruation. It is said that corpus luteum hormone secreted after ovulation is the cause, and it is also said that stress, vitamin B6, and magnesium deficiency, and the like worsen the symptom. First, do moderate exercise and take balanced diet and the like as a way in daily life to alleviate the symptom” or the like.

Note that relations among basal body temperature, a menstrual cycle, female hormone values, symptoms, and the like are known to some extent from the existing medical knowledge. Based on the medical knowledge, the present system sets a message including explanation, advice, and the like. The server 1 outputs, at an appropriate timing, a message which is based on the above medical knowledge and is appropriate for the user input information and the health state resulted from the tendency analyses and the like. The above timing is, for example, a specific time point such as a luteal phase during the menstrual cycle of the user, a time point at which a tendency and a characteristic of a change in the mind and body accompanied with an increase or a decrease in internal secretion, and values of female hormones reach predetermined values, or the like. Thus, since information such as advice is provided at an appropriate timing for the state of the user, satisfactory effects such that the user can easily understand the information, for example, can be obtained, even when the information provided is known knowledge.

A link such as a URL may be attached to the output message. The URL at that time may be not only a static URL but also a dynamically collected URL. For example, in the case of a warning of the possibility of the disease, the explanation information page of the disease is linked. For example, it is a URL collected by a certain word existing on the Internet. Moreover, in the case of the consultation recommendation, it is linked to a page where information on the treatment and the examination of the recommended subject is provided and a page for searching for medical institutions and the like and the information thereof.

[Medical Examination Information]

FIG. 8 shows a configuration example of the medical examination information 52. A table of the medical examination information 52 in FIG. 8 includes, as items, medical institution, treatment method, achievement, examination institution, examination, examination type, examination item, examination method, medical reference information, unique reference information, and the like. The medical examination information 52 includes management of the contents of the treatment and the examination provided for each medical institution and examination institution.

The “medical institution” item stores identification information and a name of a medical institution and is, for example, “medical institution A (hospital A).” The “treatment” item stores identification information and a name or names of one or more medical treatment employed by the medical institution, and is, for example, “treatment A.” The “treatment method” item stores information on the treatment method, the treatment type, and the like concerned with the treatment, and is, for example, “treatment method A” and the like.

The “achievement” item stores information such as the number of treatment cases and the number of surgery cases. The information includes, for example, the annual number of cases for timing method, the annual number of cases for artificial insemination, parameter, the number of pregnancy, the pregnancy rate, and the like.

The “examination institution” item stores identification information and a name of an examination institution which is associated with the medical institution and mainly deals with the examination, and is, for example, “examination company A.” When the examination institution and the medical institution are the same, this information can be omitted. The “examination” item stores identification information and a name or names of one or more medical examinations employed by the examination institution and is, for example, “examination A.” The “examination type” item is information indicating the type of the examination such as a blood test, a urinalysis, an ultrasound examination, and palpation, and is, for example, “blood test.” The “examination item” item is an item for the examination subject and is, for example, “luteinizing hormone (LH)” or “follicle stimulating hormone (FSH).” The “examination method” item stores identification information and a name of the examination method associated with the examination and is, for example, “examination method A=enzyme immunoassay (EIA),” “examination method B=chemiluminescent immunoassay (CLIA),” or the like.

The “medical reference information” item is information on values, ranges, and the like which are statistical criteria for determining the treatment and the examination, and is a value, so called reference value. Numerical ranges and units in the medical reference information are different depending on the examination institutions. This is because examination institutions have different examination methods, examination reagents, specimens, and the like. An example of the numerical range is “range A=value A1 to value A2 [mol/L].” The value A1 is a lower limit value, and the value A2 is an upper limit value. For example, when the LH value, which is the value of the examination item, is within the range A, the LH value is determined to be normal or good. When the LH value is outside the range A, the LH value is determined to be abnormal or bad, caution needed, or the like.

The information including the above examination methods and medical reference information is set by the present system by using information provided or disclosed by the medical institutions and the examination institutions. Moreover, when there are a plurality of treatment and examinations even in one medical institution or examination institution, the information is managed in association with each of the treatment and the examinations.

The “unique reference information” item is information on a numerical range which is set for specific control in the present system based on the “medical reference information” and is a reference unique to the present system. The numerical range is set mildly in consideration of numerical ranges of a plurality of pieces of medical reference information. The example is “range C=value C1 to value C2 [mIU/mL]” or the like. The value C1 is a lower limit value, and the value C2 is an upper limit value. For example, when the LH value, which is the value of the examination item, is within the range C, the LH value is determined to be good. When the LH value is outside the range C, the LH value is determined to be bad, or the like.

As for the numerical range of each reference information, only a threshold value or the representative value within the range may be provided. The numerical range may be set for each period. For example, a range a is set for a follicular phase, a range b is set for an ovulatory phase, and the like. The numerical range may be defined by a predetermined function. Moreover, the determination is not limited to be binary such as good/bad and may be made with a plurality of levels using a plurality of values.

The above examination methods, examination items, numerical ranges of the reference information, and the like include management of unit information. For example, there are various units such as [mol/L], [ng/mL], and [mIU/mL]. The present system appropriately performs unit conversions based on the management information.

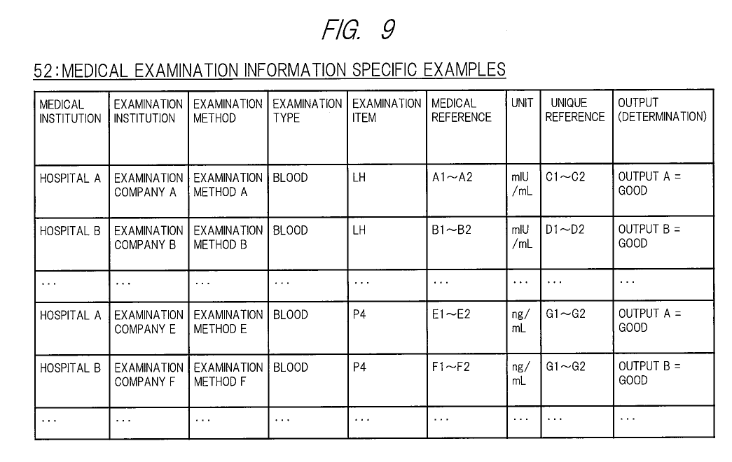

FIG. 9 shows a specific example of the medical examination information 52 in FIG. 8 and a setting example of the unique reference information. For example, the first row indicates that the hospital A employs LH measurement by a blood test with an examination method A by an examination company A. The range of the medical reference for the examination method A is A=A1 to A2 [mol/L], and the LH value is determined to be good when the LH value is within the range A. Similarly, the second row indicates that a hospital B employs LH measurement by a blood test with an examination method B by an examination company B. The range of the medical reference for the examination method B is B=B1 to B2 [ng/mL], and the LH value is determined to be good when the LH value is within the range B.

As in the above examples, the examination method A of the examination institution A (e.g., examination A) differs from the examination method B of the examination institution B (e.g., examination B), and the numerical ranges and units of the references for determining the value to be good and the like are different. Thus, the values obtained by different examination methods cannot be compared in principle when conversion equations are not established in the medical industry. However, since it is difficult for the user to interpret and understand such medical information, the user may misleadingly perform comparisons in some cases.

The present system sets and manages different information for each examination period in the above medical examination information 52. Then, the present system sets “unique reference information” in addition to “medical reference information” in association in the medical examination information 52. Upon the analysis, the present system refers to the medical examination information 52 and applies the “medical reference information” or the “unique reference information” according to the medical institutions, the examination institutions, the examination methods, and the like used for each user.

The use of the “medical reference information” is as follows. Upon the analysis, the present system identifies the medical institutions, the examination institutions, and the like used by each user from the examination result data 54 in FIG. 5 or the user attribute information 51 for each user and reads out and applies the medical reference information associated therewith from the medical examination information 52. Then, the analysis unit 16 compares the values of the examination items of the user with the medical numerical ranges and determines the state to be good or the like.

The use of the “unique reference information” is as follows. Similarly, upon the analysis, the present system refers to the examination result data 54, the medical examination information 52, and the like, reads out and applies the unique reference information associated with each examination method and examination item, compares the values of the examination items of the user with the unique numerical ranges, and determines the state to be good or the like. Note that the system according to another embodiment may manage and use only either one of the above medical reference information and the unique reference information.

A setting example of the unique reference information is as follows. The “unit” column in FIG. 9 indicates the units relating to the unique reference information of the “unique reference” column. The first and the second rows of the table in FIG. 9 show examples in which different pieces of the unique reference information are individually set. The range of the unique reference information C=C1 to C2 [mIU/mL] is set for the range of the medical reference information of the examination institution A in the first row A=A1 to A2. The range of the unique reference information D=D1 to D2 [mIU/mL] is set for the range of the medical reference information of the examination institution B in the second row B=B1 to B2. The ranges A, B, C and D are different.

In another example in FIG. 9, the same unique reference information may be set for a plurality of different examination institutions as shown in the lower rows. For example, a range G=G1 to G2 [ng/mL] is set as the same unique reference information for an examination institution E and an examination institution F. In this case, the unique numerical range that is set to be the same is set to be a mild reference for a plurality of numerical ranges of the premised medical reference information, for example, as shown below.

For the range E=E1 to E2 of the medical reference of the examination method E and the range F=F1 to F2 of the medical reference of the examination method F, the present system first converts the units to be the same. For example, when units, [pM] and [ng/mL], are present for a certain examination item, the units are unified to be [ng/mL]. Suppose the magnitude relation of the values in the converted range is, for example, E1<F1<E2<F2. For example, the present system takes a wide range E1 to F2 according to the OR condition between the range E and the range F and sets the range as the range G=G1 to G2. Alternatively, the present system may take a narrow range F1 to E2 according to the AND condition between the range E and the range F and set the range as the range G=G1 to G2.

Moreover, in addition to the values E1, F1, and the like of the medical reference, the present system may take unique values according to a technique of statistical values and the like and set the values as a unique numerical range. For example, the system may set a range X=X1 to X2 by values X1 and X2 (E1<X1<F1, E2<X2<F2).

As described above, the medical reference information and the unique reference information are set so as to deal individually and comprehensively with a plurality of users and a plurality of examination methods. In particular, the unique reference information is set as a unique mild reference by the present system. When the examination methods are different, the present system handles the examination result data 54 and the like of the user as closed data for each individual user in principle. Then, the present system applies the medical reference information or the unique reference information appropriate for the examination method for each user and performs the tendency analyses and the like.

Problems in the above medical reference information will be supplemented. When an examination institution is outsourced by a medical institution or a medical institution itself conducts an examination, the examination methods and the like may be different as previously mentioned. Even with the same examination method, the numerical ranges of the references are different because specimens, that is, samples used for statistics, reagents, devices, and the like are different, as previously mentioned. In this case, the values of the examination items have different meanings, and the comparisons cannot be merely made. Moreover, as the medicine develops, the above ideas, examination methods, and medical reference also change. For example, in reproductive medicine, there is no one standardized reference determined with regard to judgment of values of endocrinological examinations, and the like at present. There are also no conversion equations or the like provided for mutually converting values among a plurality of different examination methods and the like. Many users have difficulties understanding the above background. Thus, as means for solving the problems, the present system manages the differences in the examination institutions and the like by the medical examination information 52 and provides functions by settings of the medical reference information and the unique reference information so as to be able to cope with the above background. Therefore, support for individual users can be made.

Regarding the examination result data 54, a system according to another embodiment uses a conversion equation, which enables conversions between values of examination items of different examination methods and is established in the field of reproductive medicine (academic association, medical association, and the like) to set a conversion equation unique to the system. For example, suppose there are a value of an examination item of an examination method A of a user A and a value of an examination item of an examination method B of a user B, and these values are desired to be roughly compared with each other. The present system converts the values of the above different examination methods by using the unique conversion equation and provides the user with the converted information as helpful information. This unique conversion is useful for a schematic comparison even though the conversion is not strict conversion.

[Screen Including Information on Each User]

FIG. 10 shows a screen displaying information on each user as “MY medical record” as an example of a service screen of the present system. This MY medical record is comprehensive information indicating the health state and the like of each user as specific information of the present service and includes various information such as the user attribute information, the graphs, the calendar, and the messages of the analysis results. The present screen has a field 101 of the user attribute information, a field 102 of the body temperature-menstruation graph, a field 103 of the examination result graph, a field 104 of the calendar, a field 105 of the output messages for the analysis results, and the like.

The field 101 displays information on each attribute of the user based on the registered user attribute information 51. The right side is an example of displaying [treatment history] and [action]. The “treatment history” uses the information in the aforementioned “treatment” item in FIG. 4. The [action] shows an example of displaying main exercise, diet, and the like of the user based on the calendar input information 55, the action extraction function, and the like.

Based on the health data 53 of the DB 50, the field 102 displays the body temperature-menstruation graph of the user as in an example in FIG. 13 described later. The horizontal axis represents time (day), and the vertical axis represents the value of the body temperature. The field 102 may also display the result information on the tendency analysis of the body temperature and the menstruation together. Moreover, with a body temperature registration button, a field for registration of the body temperature data is displayed by a pop-up or the like, or a transition is made into another screen. The user can directly input the value of the body temperature or select a choice for registration in the field. Alternatively, the body temperature may be registered by plotting on the graph. Similarly, information such as a menstruation date can be registered with a menstruation registration button. For each graph, the user can designate a period for display, for example, the past one month.

Based on the examination result data 54 of the DB 50, the field 103 displays the graphs of the female hormones and the like of the blood test results of the user as in an example in FIG. 14 described later. The horizontal axis represents time, and the vertical axis represents values of examination items. The field 103 may also display the result information on the tendency analysis of the examination results together. Moreover, a field for registration of the examination result data is displayed by an examination result registration button. The user can directly input the values and the like of the examination results or select a choice for registration in the field. Furthermore, in the field 103, a selection field is provided when there are a plurality of types of examination items, and a graph of the examination item selected by the user is displayed.

In the field 104, various information such as basal body temperature, menstruation, an action, a symptom, and a note can be input and recorded for each calendar date, and the various information registered in the calendar input information 55 can be browsed. When the user performs operation to select a part where information has already been registered on the calendar date, the contents of the registered information are displayed in, for example, a pop-up, another field, or the like. The field 104 displays information including the current date and the past periods. The calendar allows the user to designate a period for display. With the calendar, the user can confirm, review, and recall various information such as basal body temperature, menstruation, actions, symptoms, and notes and can also enter and schedule various actions and plans such as hospital visit.

The field 105 displays the latest message information on various analysis results including the aforementioned tendency analyses, action extraction, and disease risk warning. The messages may be displayed not only in the field 105 but also in other fields such as a graph and a calendar and in the screen.

Each aforementioned field and each item on the screen can be set on whether to be displayed or not, where to be located, when to be used, or the like, by the user setting. For example, a certain field is in a folded state when not displayed, and the field is automatically switched to be displayed when the user wants to see and then performs a selection operation, or when a specific timing comes. In addition, the information in each field such as the graph may be displayed in parallel with the time axis such as a date. In this case, it is easy to see the correspondence relations. Since the information in each field is managed by history, the user can designate and browse the past information.

On the screen of the MY medical record above, the user can see a list of each information on his or her health state, can browse individual information, and can recognize his or her own health state easily. Other screens and fields include a HOME screen of the service of the present system, a screen for user setting, a screen for each item such as the body temperature, a screen for searching for registered information, and the like.

[Screen Including Calendar]

A screen example including the calendar is as follows. Various items such as the aforementioned menstruation and action are provided as items of information to be registered and displayed in the calendar. The Information on each item can be registered with choices, values, text, marks, and the like. The choices include choices set by the present system and choices set by the user. Formats of the calendar and information input include the followings.

(1) In a calendar of a first format, dates are aligned along the horizontal axis, and a plurality of information items are aligned along the vertical axis as shown in the example of 104 in FIG. 10. The user selects the date and information item to be input, and the information is input in the intersection of the date and the information item. In another format, the information items may be set along the horizontal axis, and the dates may be set along the vertical axis. As for the date selection, the current date is automatically selected by default.

(2) In a calendar of a second format, dates are aligned horizontally and vertically as in the example of 111 in FIG. 11, and various information items are provided in the date on a daily basis. The user selects a date to be input, thereby displaying an input field and a screen for each day. As in the example of 112 in FIG. 11, the input field for the day has a plurality of information items, and the user inputs information in each item. The user can input information in an arbitrary date all at once.

(3) A calendar of a third format does not employ a method for selecting the date as in the first and the second formats, but automatically and largely displays an information field for one day of the current date on the screen of the terminal 2 of the user. In the field for the day, a plurality of information items are included, and a message and the like are displayed. The field for the day can be transitioned into a field of a monthly basis and the like.

[Calendar Screen Example (1)]

A screen example of the calendar of the first format is as follows. For example, the treatment, the prescription, the basal body temperature, the menstruation, the action (including exercise recuperation, diet therapy, and the like), the symptom, the note, and the like are provided as information items input in the calendar date. The “menstruation” item enables registrations of a menstruation date and presence or absence of menstruation. The “treatment” item enables registrations of a visited date, a regularly visiting hospital, and the like for each treatment and examination. The “prescription” item enables registrations of the prescribed medicine or the commercial name of the medicine, the day of taking the medicine, the amount of the medicine, and the like. The “action” item enables registrations of exercise done by the user and information on diet and food. Items for other types of actions may be provided. The “symptom” item enables registrations of various symptoms, presence or absence of stress and degree of stress, and the like.

The “note” item enables registrations of texts of any notes expressing feelings, emotions, symptoms, actions, memos, and the like. For example, the user can input the text of the note in the input form and register the text by a registration button after selecting the date. Moreover, designation of the date (e.g., December 1) may be enabled in a predetermined format such as “#1201#” upon the above input. Furthermore, registrations of the basal body temperature (e.g., 36.65 degrees) and the like may be enabled in a predetermined format such as ‘#3665#.’

[Calendar Screen Example (2)]

FIG. 11 shows a screen example and an input example on a daily basis in the case of the calendar of the second format. A calendar of 111 is an example with each week aligned vertically and horizontally. The user selects a desired date, for example, a today's date, from the calendar. The input field of the selected day is displayed by a pop-up or the like. As in 112, the input field for one day includes various information items such as menstruation, body temperature, timing (timing method), treatment, examination, prescription, action (exercise therapy, diet therapy, music therapy, and the like), symptom, feeling, and notes, and the information can be input in each item by choices, text, and the like as in the case of the first format.

[Input Field of Individual Item]

The present system may separately provide a field or a screen for each individual item among the items for a plurality of information elements, for example, the above menstruation. The user can confirm the detailed information in the field or the screen for each individual item and can input the detailed information by choices with a list box and the like, text, and the like. Examples of displaying and inputting in the input fields of individual items are as follows.

The field of the calendar of 111 and the input field for one day of 112 in FIG. 11 can be transitioned into the input field of individual information items as in 113 according to the selection operation by the user. For example, when the “menstruation” item is selected, the input field of the “menstruation” item is displayed by a pop-up or the like.

In the input field of the “menstruation” item, a menstruation date and a menstrual period can be input by designating a date range. This field may display information on the last menstruation date, the last menstrual period, the current menstrual cycle, the predicted ovulation date, and the like based on the registered menstruation data and analysis results. Moreover, a transition into the corresponding graph screen can be made by a link in the displayed information. In this field, items for selectively inputting information such as an amount and quality and the like of secretions of menstruation, and items for inputting text of a note of the menstruation may be provided.

Moreover, for example, in the case of the input field of the “exercise therapy” item, a name and a type of exercise, a date, arbitrary text, and the like can be input. This field presents multiple choices for exercise and enables registration of the exercise of the choice selected by the user from among the choices. The present system sets common action choices. Moreover, for this field, the user can set the exercise that the user often does. For example, text “walking for 30 minutes” and the like can be set as the setting of the exercise A. This enables simple registration by the user since the exercise A and the like are presented as choices upon registration of information on daily exercise. In the input field of the “diet therapy” item, it is likewise possible to input names and types of diet and food, a date, arbitrary text, and the like, and the frequent diet can be set by the user.

In the input field of the “treatment” item, a name, a type, and details of the treatment, a date, arbitrary text can be input. For example, when the treatment is “artificial insemination,” information such as LH positive/negative, follicle size, and endometrial thickness can be input. When the treatment is “in-vitro fertilization,” information such as in-vitro fertilization method, egg collection method, egg collection date, the number of collected eggs, follicle size, grade, and endometrial thickness can be input. Moreover, in the case of male user's sperm collection, information such as a date, an amount, concentration, and motility can be input.

In the input field of the “examination” item, the date of the examination, the medical institution which the user regularly visits, the examination institution which conducted the examination, the examination method, the examination items, the values, and the like can be input by choices and text and registered in the aforementioned examination result data 54. Moreover, this field may enable input of information on predetermined test results such as an ovulation test and a pregnancy test. The ovulation test and the pregnancy test may be tests conducted by the user herself using commercially available test drugs and medical devices or may be predetermined examinations by a medical institution and an examination institution.

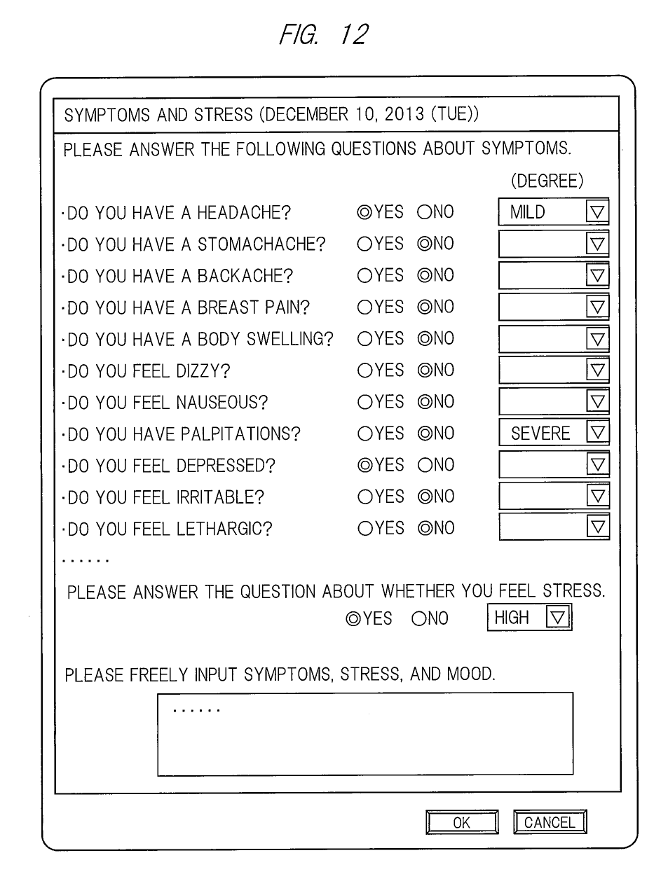

FIG. 12 shows a screen example of an input field of “symptoms and stress” item for the day. In this input field, types, presence/absence, degrees, and the like of various kinds of symptoms, stress, and the like concerned with the functions of the symptom tendency analysis and the disease risk warning can be selectively input. In the input form, text about the symptoms, stress, mood, and the like can be freely input.

Examples of the symptoms are headache, stomachache, backache, breast pain, dizziness, depression, irritability, lethargy, and the like. The input examples are (“yes”) when the user has a headache and “mild” for the degree thereof, and (“yes”) when the user is in depression and “severe” for the degree thereof. The input example also includes (“yes”) when the user is stressed out and “high” for the degree thereof. The degrees of the symptoms and the like are, for example, level 1 (mild, no hindrance to daily life), level 2 (moderate, affecting daily life), and level 3 (severe, hindrance to daily life) and the like.

The analysis unit 16 of the server 1 uses the user input information such as the symptoms and the stress on the above screen and the input and analysis results of the text of the notes to determine the health state of each user when performing the processing such as the disease risk warning. The analysis of the text of the notes indicates extraction and analysis of words relating to the symptoms, stress, feelings, and the like included in the text of the notes. For example, it is possible to determine good/bad of the health state and the like of the user based on the words such as “feel bad” and the number thereof.

The input field in FIG. 12 is an example of enabling exhaustive input of various symptoms for general checks on a plurality of diseases. The input field is not limited to this, and a screen for checking each specific disease, an input field for each specific symptom, and the like may be provided. Moreover, the present system may automatically determine the timing of the display to display the input field. When the health state of the user is in a specific state, for example, when the health state corresponds to the luteal phase, the server 1 automatically displays an input field for checking a specific disease, phenomenon, symptom, and the like (e.g., PMS) and encourages the user to input. The user may only input the information each time the input field is displayed.

[Body Temperature-Menstruation Graph]

Next, FIG. 13 shows an example of the body temperature-menstruation graph. The horizontal axis represents the number of days, and the vertical axis represents the values of the basal body temperature. Reference character a1 indicates a menstruation date (so-called a menstrual period date) and a menstrual phase which is the duration of the menstruation. Reference character a2 indicates a menstrual cycle and is the number of days from the last menstruation date to the next menstruation date. Reference character a3 indicates a predicted ovulation date. Reference character t1 indicates a low temperature phase or a low temperature period in which the basal body temperature is relatively low. Reference character t2 indicates a high temperature phase or a high temperature period in which the basal body temperature is relatively high. Reference character a4 indicates a temperature difference ΔT between the low temperature phase t1 and the high temperature phase t2. The temperature difference ΔT is, for example, a value calculated uniquely by the present system by using a difference between the maximum body temperature in the high temperature phase t2 and the minimum body temperature in the low temperature phase t1.

Each phase of the follicular phase t3, the ovulatory phase t4, and the luteal phase t5 is shown in the menstrual cycle a2. Around the ovulatory phase t4 and the predicted ovulation date a3 are the period of easily getting pregnant. Note that information on internal secretions of female hormone and the like in each period and information on the effects to the mental and physical may be displayed upon the display of the body temperature-menstruation graph.

[Examination Result Graph]

FIG. 14 shows a graph of LH and FSH of the female hormones, which are the examination items for the blood test of a healthy person, as an example of the examination result graph. The horizontal axis represents the number of days, and the vertical axis represents female hormone values. Incidentally, FIG. 14 shows the graph in connection with each period of t3 to t5 in FIG. 13. For example, among the blood test items, LH, FSH, E2, P4, AMH, and the like are deeply involved in pregnancy and the like. The present system handles a plurality of these types of examination results individually and comprehensively.

A luteinizing hormone (LH) is a hormone which promotes ovulation and corpus luteum formation and thus can be used for ovulation prediction. Reference numeral 141 is a polygonal line of the LH value, and a temporary peak, that is, the maximum value of the LH occurs in the vicinity of the ovulatory phase t4 as shown in FIG. 14. The vicinity of the peak day of the LH corresponds to the ovulatory phase t4.

A follicle stimulating hormone (FSH) is a hormone which promotes follicle development. As the age gets older, the value of the FSH tends to be higher. Therefore, the FSH can be, for example, a judgement factor for continuation of in-vitro fertilization. Reference numeral 142 is a polygonal line of the FSH values. The FSH similarly has its peak in the vicinity of the LH peak. The unit of LH and FSH is, for example, [mIU/mL].

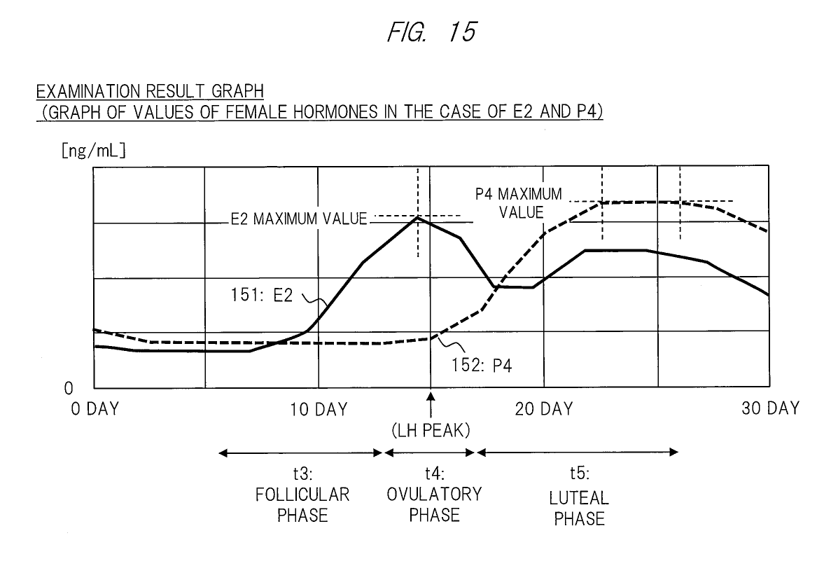

FIG. 15 similarly shows an examination result graph of E2 and P4 of the female hormones. Estradiol (E2) is a kind of estrogen (follicle hormone) and has functions such as maintenance of reproductive function, follicle maturation, stimulation of ovulation, and endometrial proliferation. The value of the E2 rises when the follicle grows up, and the value of LH rises when the value of the E2 reaches a certain value and acts on the pituitary gland. Therefore, to support pregnancy, the E2 is useful for ovulation prediction since the E2 enables an earlier grasp of ovulation tendency than the LH observation. Reference numeral 151 is a polygonal line of the E2 values. The E2 rises before the vicinity of the LH peak in the ovulatory phase t4 and in the luteal phase t5. The unit of the E2 is, for example, [ng/mL].

Progesterone (P4) is also called corpus luteum hormone. The P4 suppresses follicular growth, thickens the endometrium and acts on continuation of pregnancy. Reference numeral 152 is a polygonal line of the P4 values. The P4 rises in the luteal phase t5. The unit of the P4 is, for example, [ng/mL].

An anti-Mullerian hormone (AMH) is a female hormone secreted from the follicle, and it is said that the function of the ovary can be estimated from the AMH value. A graph is similarly created also for the AMH. The female hormones are not limited to the above five types, and various types of other female hormones such as prolactin (PRL) and testosterone can be also similarly applicable. The examination results can be applied not only to the above female hormones, but also to other chemical substances and index values.

The values of the above body temperature, menstruation, female hormones, and the like, the states of the variations thereof, and the health state are medically relevant. Based on medical knowledge, the present system sets medical reference information and unique reference information on values of a plurality of elements including the above body temperature, menstruation, and female hormones and, by using these, analyzes the health state including the relevance and tendency of each element. As in the examples of the graphs, on the screen, the user can browse the states of his or her body temperature, menstruation, and female hormones and the like of the examination results together with the messages for the analysis results.

[Tendency Analysis]

Next, the details of the processing for the tendency analyses will be described using FIGS. 1, 8, 13, and the like. The analysis unit 16 in FIG. 1 performs the tendency analysis processing relating to the body temperature, the menstruation, the examination results, the actions, the symptoms, and the like of each user while referring to the medical examination information 52 in FIG. 8 and the processing definition information 58 in FIG. 1. In the tendency analyses, the analysis unit 16 compares the values of the data of the user, such as the graph in FIG. 13, with, for example, the numerical ranges of the unique reference information in FIG. 8 or a reference graph described later, and determines and detects the state of being good or bad in the absolute values and the tendencies of relative numerical variations in time series, for example, the state such as improvement, deterioration, maintenance, and the like. Then, the analysis unit 16 decides an output message appropriate for the state of the user.

The analysis unit 16 calculates values of predetermined items such as the menstrual cycle a2 and the body temperature difference ΔT in FIG. 13 mentioned above, calculates the amounts of variation of the values in time series for each item, and records the values and the amounts of variation in the analysis information 56 in FIG. 1. The analysis unit 16 calculates, as the amount of the variation, the differences between the values of the items such as the plurality of menstrual cycles a2 and the body temperature difference ΔT in consecutive periods before and after the menstrual cycles a2. The analysis unit 16 determines the state of the tendencies such as improvement, deterioration, and maintenance while comparing the amount of the time series variation of the values of the above-predetermined items with the predetermined values relating to the variation based on the processing definition information 58.

The analysis unit 16 determines the periodic stability in the above time series values of the data of the user and the variation thereof. For example, the analysis unit 16 refers to the variation in the items such as the menstrual cycle a2 of the user in the past period, determines that the periodic stability is high and the state is good when the variation is small, and determines that the periodic stability is low and the state is bad when the variation is large.

The analysis unit 16 determines graph patterns from, for example, the above time series values of the data of the user. The analysis unit 16 may detect specific periodic graph patterns in the graphs of the body temperature, the menstruation, the examination values of the female hormones and the like shown in FIGS. 13 to 15. The analysis unit 16 compares, for example, the values of the graphs of the user with the numerical ranges of the unique reference information or the reference graph described later and determines and detects whether the comparison results correspond to the above graph patterns and the like.

The analysis unit 16 may compare the above values of the user with the numerical ranges of the unique reference information and, based on the differences and the like thereof, calculate and determine proximity or a degree of similarity between the values of the user and the values of the references. The analysis unit 16 may similarly calculate and determine the proximity or the degree of similarity between the graphs of the user and the reference graphs.

Moreover, the analysis unit 16 may calculate and record statistical values such as average values of the values of the items such as the plurality of the menstrual cycles a2 of the user in time series. Furthermore, in the tendency analyses, the analysis unit 16 may compare the values of the items such as the menstrual cycle a2 of the user with the above statistical values of the same item of the same user in the past to determine the tendencies.

The present system provides the user with information on the tendencies such as improvement, deterioration, and maintenance, magnitude of the variations, the degrees of the improvement and the like, the periodic stability, the patterns, the statistical values, the proximity or the degree of similarity with the references, and the like according to the results of the above tendency analyses. Note that the values of the body temperature and menstruation have individual differences and the values even of the same person vary according to stress and the like. As described above, the present system conducts the advanced analyses for recording and determining the states of the body temperature, the menstruation, and the like for each user, including the tendencies of the time series variations.

[Tendency Analysis of Body Temperature and Menstruation]

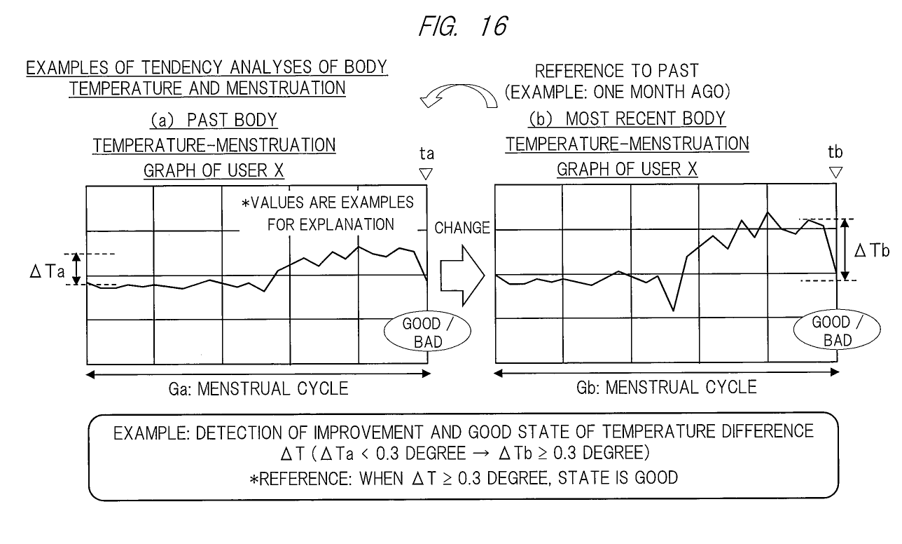

An example of the tendency analyses of the body temperature and the menstruation will be described with reference to FIG. 16. (a) in FIG. 16 shows a past body temperature-menstruation graph of a certain user X in one menstrual cycle. (b) in FIG. 16 shows a current body temperature-menstruation graph of the same user X in one menstrual cycle. Note that the values of the graph are just examples for explanation.

The analysis unit 16 in FIG. 1 creates a body temperature-menstruation graph like the one shown in FIG. 13 from the health data 53 of the user X by using the graph creation unit 14 and acquires or calculates the value of each item such as the menstrual phase a1, the menstrual cycle a2, the predicted ovulation date a3, the low temperature phase t1, the high temperature phase t2, the follicular phase t3, the ovulatory phase t4, the luteal phase t5, the temperature difference ΔT, and the maximum and minimum values of the body temperature. The analysis unit 16 analyzes the tendency of a change in the time series values in the body temperature-menstruation graph from the past to the present. For example, the analysis unit 16 refers to the body temperature in the most recent menstrual cycle Gb of the current date tb in (b) and the body temperature in the closest menstrual cycle Ga of the past date ta in (a).

As a way of referring to the past information in the target period of the tendency analyses, for example, the analysis unit 16 refers to the past date ta by dating back by the number of predetermined days or the like from the current date tb. Then, the analysis unit 16 sets, as the target period, a period between the past date ta and the current date tb, a period between the closest menstrual cycle Ga of the past date ta and the most recent menstrual cycle Gb of the current date tb, or the like. The number of days for dating back and the like are set according to individual processing in the processing definition information 58, and for example, three days ago, one week ago, one month ago, three months ago, one year ago, one menstrual cycle ago, three menstrual cycles ago, or the like can be set. Moreover, the target period can be set to a specific phase such as the luteal phase t5.

The analysis unit 16 determines changes in values of the body temperature in the periods of, for example, the time series consecutive menstrual cycles Ga and Gb. The analysis unit 16 compares the value of the temperature difference ΔT of the user with 0.3 degree, which is the reference value of ΔT in the processing definition information 58 in FIG. 1, and determines that the state is good when ΔT is 0.3 degree or more. In the example in FIG. 16, regarding the temperature difference ΔT, the temperature difference ΔTa in (a) is smaller than 0.3 degree, and the temperature difference ΔTb in (b) is 0.3 degree or more. That is, the body temperature and the temperature difference ΔT can be determined to correspond to a so-called two-phase pattern state since the ΔTa in (a) was not in a very good state and the ΔTb in (b) has improved to a good state. When the good and improved state of the body temperature and the temperature difference ΔT is detected, an output example is the output ID=001 in FIG. 7 mentioned above. Another example is “the temperature difference ΔT was XX degrees in the last menstrual cycle and XX degrees in the current menstrual cycle, increased by XX degrees, and improved to a good state of 0.3 degree or more.”

Moreover, even when the current temperature difference ΔTb is in the state of satisfying the relation ΔTb<0.3 degree, the analysis unit 16 may determine the improvement when the amount of the variation (ΔTb−ΔTa) of ΔT in a predetermined period from the past to the present is a certain amount (ΔTx) or more and relatively approached the state of satisfying the relation ΔT≧0.3 degree. The above ΔTx is a set value in the processing definition information 58. That is, when the relations ΔTa<0.3 degree, ΔTb<0.3 degree, and (ΔTb−ΔTa)≧ΔTx are satisfied, the analysis unit 16 may determine the improvement. The output example is “the temperature difference ΔT has approached a good state (two-phase pattern)” or the like.

Similarly to the above, the analysis unit 16 compares the values of the body temperature, the menstruation, and the examination results, and the calculated values of the predetermined items such as the menstrual cycle a2 with corresponding reference set values and determines and detects whether the absolute values are good or bad and tendencies such as relative improvement, deterioration, and maintenance. The determinations of the tendencies in a period including three or more consecutive menstrual cycles a2 are also possible. The output example is “in the period of the past X months, the values of the body temperature, the menstruation, and the female hormones have improved,” “physical condition and rhythm are good and stable,” or the like.

[Tendency Analyses of Examination Results]

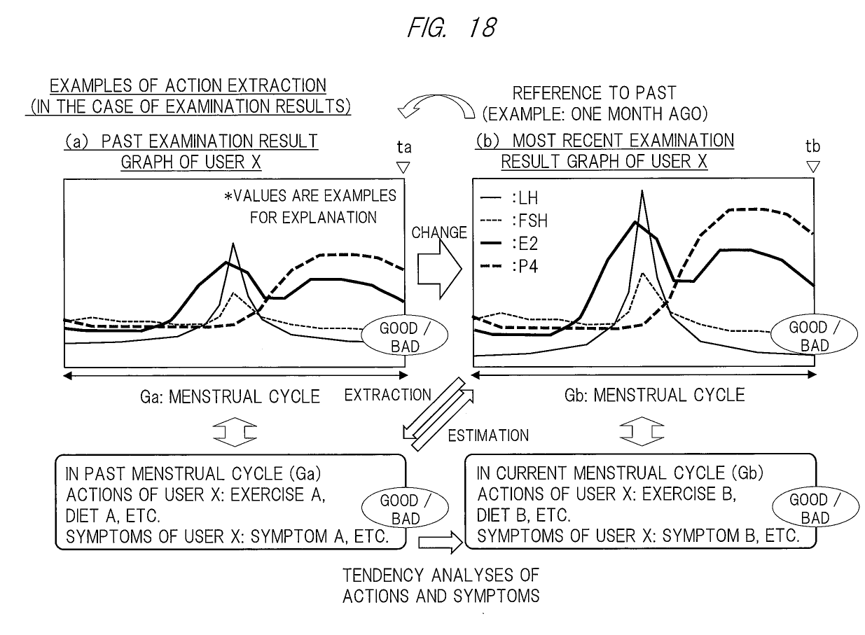

Example of the tendency analyses of the examination results will be described with reference to FIGS. 16, 18, and the like. In the tendency analyses of the examination results, the analysis unit 16 individually determines, regarding the time series values of, for example, the plurality of types of female hormones LH, FSH, E2, P4, and AMH of the examination items of the examination result data 54, whether the values are good or bad, and the tendencies such as improvement of the values, referring to the reference information and the like of the medical examination information 52 in FIG. 8. This individual processing is basically possible similarly to the tendency analyses of the body temperature and the menstruation. The analysis unit 16 determines the maximum and the minimum values of various female hormones as in FIGS. 14 and 15, and the corresponding dates, periods, and the like. Moreover, the analysis unit 16 determines an increase and a decrease, an amount of variation, periodic stability, a pattern and the like of the values of each female hormone in a target period including a plurality of consecutive menstrual cycles a2 and the like.

A reference numerical range is set for each examination item such as the LH and for each period such as the luteal phase t5 in the aforementioned medical reference information or unique reference information. For example, the analysis unit 16 determines that the LH is good when the LH value is within the reference range, and determines that the LH is not good when the LH value is out of the range. The FSH and the like are also determined in the same way. Moreover, the analysis unit 16 determines a peak day when the value or the amount of an increase in the LH or the like exceeds a predetermined value. Furthermore, the analysis unit 16 calculates the day of the maximum or the minimum value of the LH or the like, the number of days in which the state where the value exceeds the predetermined value continues, the number of days in which the state where the value falls below the predetermined value continues, and the like. As described above, the analysis unit 16 calculates the determination results such as good/not good for each certain period and determines the tendencies such as improvement by comparing the results.

Further, the analysis unit 16 determines the comparison result between, for example, each female hormone value of the plurality of female hormones and the unique reference information. For example, when a value of a first examination item is within a first numerical range and a value of a second examination item is within a second numerical range, the analysis unit 16 determines that the value is in a good state and when the varied value of the first examination item approaches the first numerical range and the varied value of the second examination item approaches the second numerical range, the analysis unit 16 determines that the value is in an improved state.

In addition, the analysis unit 16 determines magnitude relations among the plurality of the examination items, the body temperature, and other observation items such as BMI, and the relations (including an order, an interval, and the like) among the peak days or the days of maximum values of the body temperature and the examination items. The analysis unit 16 performs time series comparison of these and performs the comprehensive tendency analysis for determination. For example, the analysis unit 16 refers to a state where the LH and FSH values are within the ranges of the unique reference information at present although the LH and FSH values were out of the ranges of the unique reference information in the past, and there is a tendency of improvement although there was a disease risk.

Based on this state, the analysis unit 16 determines the health state of the user. Example of the determinations are that each female hormone is in a good state in the follicular phase t3 and the ovulatory phase t4 during the menstrual cycle a2, some female hormones are not in a good state in the luteal phase t5, the temperature difference has changed into a state of satisfying the relation ΔT<0.3 degree, and the like.

The analysis unit 16 comprehensively determines the health state of the user from the results including the determinations of the above various states and decides the output message. The output example is “the LH and FSH values are good. The E2 and P4 values are somewhat poor in the luteal phase, but the temperature difference ΔT has slightly improved” or the like.

[Action Extraction (1)]

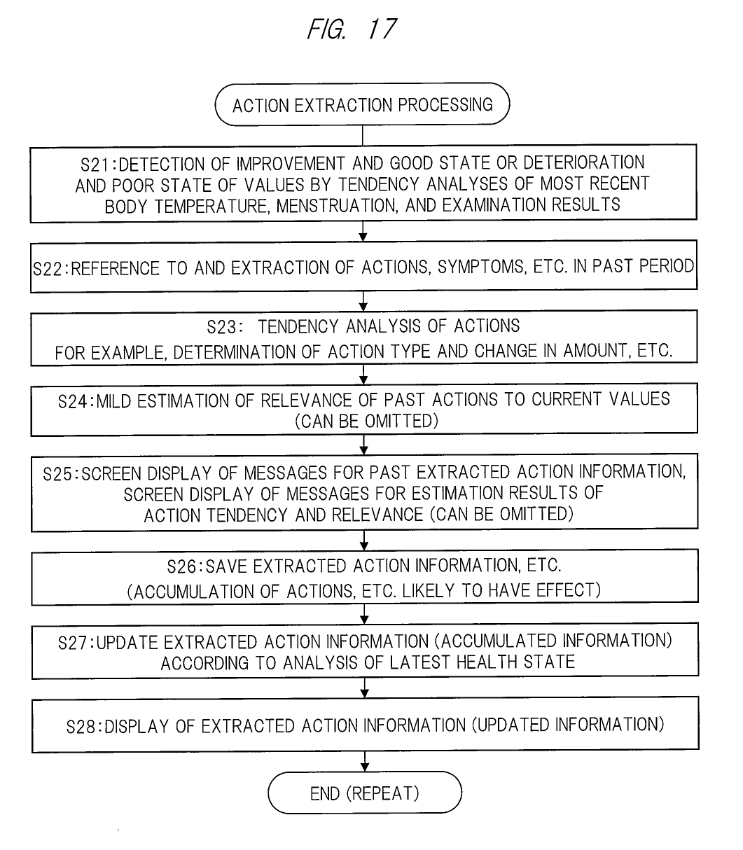

Next, the processing of the action extraction function will be described with reference to FIGS. 17, 18, and the like. FIG. 17 shows a flow of the processing concerned with the action extraction by the analysis unit 16.

(S21) The analysis unit 16 detects improvement and good state of the values or deterioration, poor state, and the like of the values based on the user's most recent data of the body temperature, the menstruation, the examination results, and the like.

(S22) Based on the above detection results, the analysis unit 16 refers to and searches for the action data, the symptom data, and the like of the calendar input information 55 and extracts information on main actions, symptoms, and the like of the user in the past period. In the same way as mentioned above, the set values of the processing definition information 58 are used for the target period of the reference, the number of days dating back from the present to the past, and the like, upon the step S22.

(S23) In the step S23, the analysis unit 16 performs the action tendency analysis processing. In the action tendency analysis processing, the analysis unit 16 determines an amount, frequency, continuity, changes thereof such as an increase and a decrease of each type of actions of the user in the target period in time series. For example, the analysis unit 16 calculates the amounts of various actions by the number of registered days.

The analysis unit 16 extracts information including at least one of life habits including actions assumed to be medically relevant and life habits including frequent actions in the past period.

(S24) In the step S24, the analysis unit 16 may estimate the relevance and the influence of the user's past actions to and on the current states of the values by using the above results of the steps S21 to S23 although the estimation can be omitted. An example of the estimation is that, when the improvement of the temperature difference or the like is detected in a period from the past to the present and an amount of a specific exercise A is maintained at or increased to a certain amount or more in the same period, the corresponding action is assumed to be influencing the current improved state. Another example is that, when the specific exercise A has changed into exercise B in the target period, the corresponding action is assumed to be influencing the current improved state. Alternatively, when the amount of the exercise A has been changed, the amount of the corresponding action is assumed to be influencing the current improved state.

Another example of the estimation is that determination can be made by also taking the symptoms and the like into consideration for the actions. For example, when there is a decrease in the degree of a symptom A such as stress, a change from the symptom A such as specific stress to a symptom. B such as relaxation or positive mood, and the like in the target period, the relevance and the influence of the past action mentioned above to and on the improvement of the current health state are determined to be deeper.

(S25) The analysis unit 16 decides the output message information based on the results up to the step S24 and causes the message output unit 17 to display the information on the screen. The output message is information such as the past extracted actions by the step S22, the action tendencies by the step S23 and the estimation results of the relevance and the influence of the past actions to and on the current state by the step S24.

(S26) The analysis unit 16 saves the result information up to the step S25 as a part of the analysis information 56 in the DB 50. As a result, the analysis unit 16 accumulates information on the actions that are likely to have an effect such as improvement for the user and conversely the actions that are likely to have no effect such as improvement.

(S27) Thereafter, the analysis unit 16 similarly continues the processing such as the above action extraction for a certain period or longer and updates the accumulated information on the above actions and like which are likely to have an effect such as improvement. The contents of the accumulated information are modified according to the health state of the user. For example, when the action A that is likely to have an effect of improvement is registered but no improvement or the like is observed in the analysis results of the latest health state, the analysis unit 16 assumes that the action A seems to have no effect of improvement and then updates the accumulated information. The accumulated information is kept as history data.

(S28) The analysis unit 16 displays, for example, information on the actions and the like, which are likely to have an effect of improvement, for example, as a message on the screen of the user at any time by using the accumulated information on the above extracted actions. Moreover, the analysis unit 16 may regularly execute the processing such as the above action extraction, not only upon the detection of the state of improvement or the like. For example, the analysis unit 16 may generate and output the information on the summery of the actions in the past one month every month.

In addition, the analysis unit 16 may output a message regarding recommended actions according to the health state of the user. This can be done by relating the recommended actions to the states of the body temperature, the menstruation, the symptoms, and the like and then setting candidates of the recommended actions appropriate for the states of the body temperature, the menstruation, the symptoms, and the like in the processing definition information 58 or the like. For example, depending on a type and a degree of the symptom, various actions such as bathing and ingestion of vitamins are recommended.

[Action Extraction (2)]

FIG. 18 shows a processing example of the above action extraction. In FIG. 18, the example will be described with the female hormones (LH, FSH, E2, and P4) of the examination result graph. (a) in FIG. 18 shows the past examination results of the user X in one menstrual cycle. (b) in FIG. 18 shows the current examination results of the user X in one menstrual cycle. The analysis unit 16 refers to the values of the examination results in the most recent menstrual cycle Gb of the current date tb in (b) and the values of the examination results in the closest menstrual cycle Ga of the past date ta in (a).

The analysis unit 16 detects changes in values of the examination results from (a) to (b), for example, a state where the LH value is good, improved, and the like. Suppose the values in (a) were not good compared with the references, but the values in (b) were improved to good values within the reference ranges. The analysis unit 16 refers to and extracts information such as actions and symptoms in the past period, which are assumed to be relevant to the current state of the user X, at the timing of the detection of the above improvement. For example, the analysis unit 16 dates back to the past date ta, which is one month before or the like, from the current date tb and refers to the data of the actions and the symptoms in the target period including the current menstrual cycle Gb and the past menstrual cycle Ga. For example, in the past menstrual cycle Ga in (a), the exercise A, the diet A, and the like are registered as the actions of the user X, and the symptom A and the like are registered as the symptoms. Also, in the current menstrual cycle Gb in (b), the exercise B, diet B, and the like are registered as the actions of the user X, and the symptom B and the like are registered as the symptoms.

Therefore, the analysis unit 16 extracts the exercise A, the diet A, and the symptom A in the above menstrual cycle Ga and the exercise B, the diet B, and the symptom B in the menstrual cycle Gb, and the like as the past actions and symptoms assumed to be relevant to and influencing the state of improvement of the LH value. The analysis unit 16 causes the information on the extracted actions and symptoms to be output. The output example is the aforementioned output ID=004 or the like in FIG. 7.

The analysis unit 16 may perform the tendency analyses of the actions and the symptoms before the extraction to decide the output extraction information or may perform the tendency analyses of the actions and the symptoms after the extraction to decide the output extraction information. For example, based on the tendency analyses, the analysis unit 16 extracts particularly exercise and diet with large amounts, severe symptoms, and the like from the actions and the symptoms once extracted. Moreover, the analysis unit 16 may determine changes from the actions in the past menstrual cycle Ga to the actions in the current menstrual cycle Gb to extract the action with a specific change.

The above examples of the action extraction and estimation are of the case of improvement, but can be also applied to the case of deterioration. Furthermore, the analysis unit 16 may perform the above action extraction and estimation processing in the same manner with a combination of the elements such as the body temperature, the menstruation, the examination results, the action, the symptom, the stress, and the feelings.

With the function of the above action extraction, the user can see the extracted information on his or her past actions and the like and the messages for the analysis results thereof on the screen and can use them as references for his or her future actions and the like. The user can easily recognize the relevance and the influence of his or her past actions and the like to and on the current state. Therefore, for example, the user recognizes good results such as improvement by good actions, and this will encourage future actions. The user also recognizes the results such as deterioration by actions which are not good, and this will be a caution for future actions. The user can easily search for exercise and diet suitable to himself or herself.

[Disease Risk Warning]

Next, the processing of the disease risk warning function and the processing of the tendency analyses of the relevant symptoms, and the like by the analysis unit 16 will be described. The analysis unit 16 mildly estimates and checks the possibilities and the like of various women's diseases in conjunction with the results of the aforementioned tendency analyses and the like of combinations of the elements such as the user's body temperature, menstruation, examination results, actions, symptoms, and notes. In the processing definition information 58, processing logic and references for the disease and the like of the check subjects are set. Note that the present analysis is mere unique mild estimation, not a medical diagnosis by a medical institution, and the output is helpful information. The user is notified of this intention.

Hereinafter, an example of a specific disease risk warning will be described with an example in which disease A=premenstrual syndrome (PMS) will be described. Note that the PMS is a syndrome in which symptoms of various bad physical and mental conditions (e.g., FIG. 12) occur prominently in the luteal phase (a period from ovulation to a menstrual period). The PMS is said to be due to various causes such as stress, fatigue, exercise, and diet. For example, the causes include lack of nutrients such as vitamins, overeating, lack of exercise, intense exercise, and the like. It is said that the state of secretion of a specific female hormone and the state of a specific symptom are relevant to the PMS. General improvement actions and general deterioration actions are known for the PMS, and it is possible to try a method to alleviate symptoms in daily life. Moreover, since the PMS has individual differences, trials and observations are useful.

The analysis unit 16 refers to the values of the “disease” and the “anamnesis” in the user attribute information 51 of the user, confirms the corresponding current or past states of the “PMS” and the like, and also confirms corresponding states of other diseases relevant to the “PMS.” The analysis unit 16 also refers to the symptom data of the user registered in the calendar, the screen in FIG. 12, and the like, and determines and detects whether the symptoms are good or bad and whether the tendencies of the symptoms are improved, deteriorated, or the like based on the tendency analyses of the symptoms, including determination of an increase or a decrease in frequency of various symptoms. In addition, the analysis unit 16 refers to the data of the body temperature, the menstruation, the female hormones, and the like, which are considered to be relevant to the “PMS,” and similarly performs the tendency analyses. Then, while referring to the processing definition information 58 relating to the check of the disease A=PMS, the analysis unit 16 mildly estimates the possibility of the disease Abased on the health state including the tendencies of the symptoms of the user.

The analysis unit 16 inputs, for example, information such as the menstruation date, the menstrual cycle, the exercise, the diet, the symptoms, the stress, and the feelings expressed in the notes in the health data of the user X. Using the input information, the analysis unit 16 determines the possibility that the user has the disease A, based on the set values of the unique reference information and the like of the processing definition information 58. The processing definition information 58 includes set values of specific symptoms and actions assumed to be relevant to the specific disease A, and set values of the values of the body temperature, the menstruation, and the female hormones in the case of the disease A.

The analysis unit 16 refers to the data of the symptoms and the stress in a target period based on the set values of the processing definition information 58, for example, a period from two weeks before the menstruation, the past one month, or the like, and extracts specific symptoms and stress appearing with a certain degree or more. For example, a symptom a with a mild headache, a symptom b with a deep depression, high stress, and the like in FIG. 12 are extracted. Likewise, the analysis unit 16 may extract the relevant actions and the like in the target period. For example, exercise a, diet a, and the like are extracted.

The analysis unit 16 compares the above specific symptoms and actions with the medical or unique reference information of the processing definition information 58 and determines the presence or absence of the possibility of the disease A, the percentage (%), or the like. For example, when the number of the above extracted specific symptoms exceeds N, which is a predetermined threshold value, or when the degrees of the specific symptoms exceed predetermined degree values, the analysis unit 16 determines that the possibility of the disease A is present. The analysis unit 16 may also determine that the possibility of the disease A is present when the total value of the degrees of a plurality of specific symptoms exceeds a predetermined value. The analysis unit 16 may also determine the level or the percentage of the possibility of the disease A by using the calculation of the symptom values and a plurality of threshold values.

The analysis unit 16 similarly performs the above check processing for various diseases B, C, D, and the like. Moreover, the analysis unit 16 can similarly determine the possibility of pregnancy and infertility by using data such as body temperature, menstruation, and examination results in combination, in the same manner as the above disease risk warning processing.

The analysis unit 16 decides the output message based on the results of the above disease risk warning processing. For example, when the possibility of the disease A is present, the output example is the aforementioned output ID=003 or the like in FIG. 7. The output information includes a warning indicating the presence of the possibility of the disease A, explanation information of the disease A, specific treatment and examination considered to be effective as countermeasures against the disease A, consultation recommendation with a medical institution or the like, for example. Moreover, the output information includes recommendation information on a specific action considered to be effective as a countermeasure against the disease A, and recommendation information on a specific product. The recommended actions may be advice on actions based on medical knowledge or may be actions such as exercise and diet based on the extraction results of the actions of the user. Note that the warning is a mild alert.

[Comprehensive Determination]

As another example of processing concerned with the disease risk warning, the analysis unit 16 comprehensively determines the health state of the user based on a combination of the values of each element including the aforementioned body temperature, menstruation, examination results, symptoms, and actions of the user, and decides the output according to the health state. The message output unit 17 displays a message such as the explanation and interpretation of the health state and the advice according to the health state.

Based on the results of the tendency analyses and the like, the analysis unit 16 grasps the temperature difference ΔT, the variation of the values of various female hormones, and the variation of the accompanying stress and symptoms for each menstrual cycle a2 in time series. The analysis unit 16 comprehensively determines the health state, comparing the states of the elements including the body temperature, the menstruation, the female hormones, and the symptoms with the set values based on the processing definition information 58.

For example, suppose, in the luteal phases t5 of the consecutive menstrual cycles a2 of the user X in FIG. 13, a change in which the value of P4, which is a specific female hormone, reaches a predetermined threshold value or more and a transition from a severe symptom a to a mild symptom b are detected. In this case, the output example is “the P4 value of the user X in the luteal phase rose in the current menstrual cycle compared with the last menstrual cycle, and a tendency of improvement is observed. In addition, accompanying this, in the period from the last to the current menstrual cycles, the severe symptom a has transitioned into the mild symptom b, and the symptom has improved,” “the symptoms a and b are symptoms of the PMS that tend to appear in the luteal phase. If you are concerned, taking examination a and the like and action a and the like are recommended,” or the like.

As described above, the present system provides not only general medical knowledge and advice, but also auxiliary messages to support judgement and analyses by the user based on the results of the comprehensively determination of the health state including the tendencies of a combination of a plurality of elements in time series for each user.

[Reference Graph]

Next, functions and processing relating to the aforementioned reference graphs will be described. The present system sets in advance, as the reference graphs, the graphs of the body temperature, the examination results, and the like corresponding to the numerical ranges of the medical reference information or the unique reference information. The reference graphs may be created by, for example, curves and polygonal lines based on representative values, regions of curves and polygonal lines having a width according to the numerical ranges of the references, and the like. The reference graphs are helpful or guidance information for the user.

The server 1 may display the reference graphs on the screen of the terminal 2 of the user for comparison with the graphs of the user. For example, the reference graphs and the graphs of the user may be displayed in parallel or in an overlapping manner. Thus, the user can see the shapes of the graphs of his or her body temperature, female hormones, and like in comparison with the shapes of the reference graphs and can easily understand whether the values of the user are close to the references, and the like.

A plurality of types of reference graphs may be set, including patterns of healthy and normal cases, patterns of cases where there is a possibility of disease or abnormality, and the like. By comparing the graphs of the user with the reference graphs in the cases of diseases and the like, it is easy to estimate and detect a possibility of disease of the user, and the like.

[Graph Interpolation Function and Graph Matching Function]

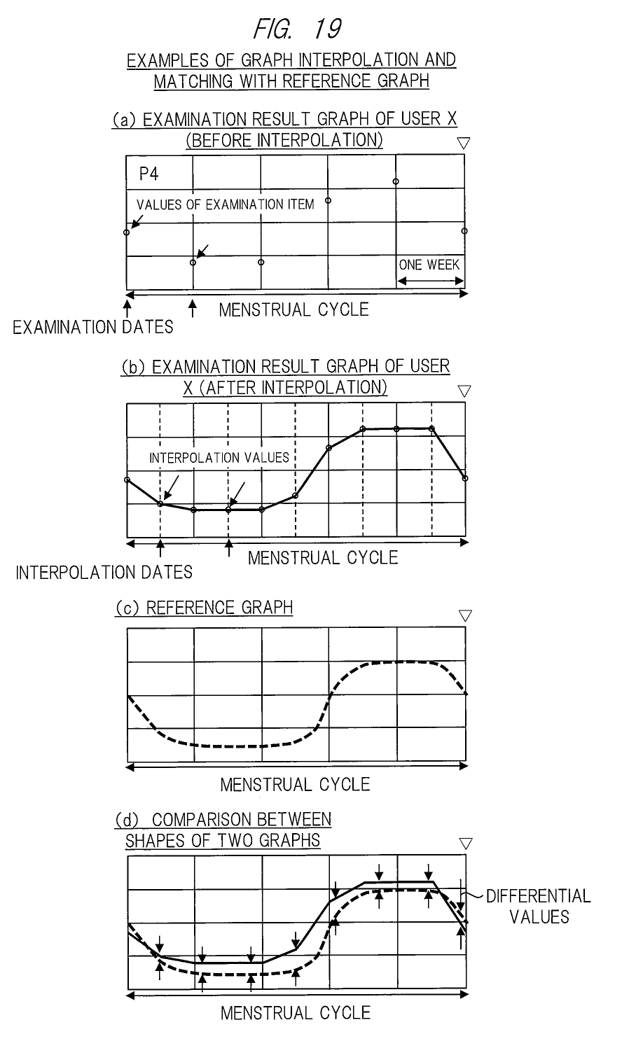

FIG. 19 shows a processing example using the graph interpolation function and the graph matching function of the server 1 and indicates an example of a graph of the female hormone of the examination results. The graph interpolation processing by the graph interpolation function is processing of interpolating a shape and data of the graph of the user. The graph matching processing by the graph matching function is processing of matching the shape of the graph of the user and a shape of the reference graph.

(a) in FIG. 19 shows an example of the examination result graph of a specific examination item of a certain user X in one menstrual cycle. Points indicate the values of the examination item. In the input examination result data 54 in this graph, the dates of the examination and the registration have certain intervals or more, and the values are intermittent. Herein, for explanation, suppose only values of the examination which is conducted once a week are registered. This registration interval is not actually the same and may be longer or shorter.

The graph interpolation function performs processing of interpolating values of non-registered dates for the data of the graph of (a). (b) in FIG. 19 shows a graph after interpolating the graph of (a). For example, dates for the interpolation are taken between each examination date in (a) as shown by the broken lines, and the interpolation values are taken on the dates for the interpolation so as to connect between the values of the registered examination dates. These interpolation values are taken in such a way that the user graph has a smoother curve than before the interpolation. The graph after the interpolation in (b) becomes smoother than the graph before the interpolation in (a) and easier to be seen. The example of (b) has a polygonal line, but may have a curve. A known technique such as a Bezier curve can be used for the above interpolation processing.

The same can be applied to the case of an interpolation graph in a period including a plurality of menstrual cycles and the case of a graph of the body temperature and the like. Moreover, the interpolation processing may be performed by using the reference graph as a reference. Furthermore, the interpolation values are only for display assistance, are estimated values, and are handled separately from the values of the actual examination results.

With the above graph interpolation function, the user can see the interpolation graph corresponding to the intermittent recording even when the user cannot record the data daily or regularly, and the shape of the graph is easy to be seen even when registered data is few. The interpolation graph can be also said to be an estimated graph of the case where registered data is many.

(c) in FIG. 19 shows an example of a reference graph relating to the examination result graph of the specific examination item in (a). The reference graph of (c) is constituted by a smooth curve.

(d) in FIG. 19 shows an example in which the shapes of the graphs are compared and determined by overlapping the reference graph of (c) and the graph of the user after the interpolation in (b) by the graph matching function. The graph matching function overlays the shape of the user graph as shown in (b) with the shape of the corresponding type of the reference graph as shown in (c), compares the shapes and the values, determines proximity of the shapes of both graphs in values, and outputs the results thereof. An index value representing the proximity of the shapes of the above graphs is set as a degree of similarity.

Based on the above data of the user graph and the information on the menstrual cycle and the like, the server 1 fetches the data of the graph portion in the comparison target period, for example, the time series values in the one most recent menstrual cycle, for example, the data of (a) or (b). Herein, the data of the graph after the interpolation in (b) is used. The server 1 fetches the above data of the reference graph of the type corresponding to the user graph, for example, the data of the reference graph of (c). As shown in (d), the server 1 overlaps the user graph in the target period with the corresponding reference graph. At this time, when the periods of the menstrual cycles and the like of both graphs are different, the periods may be adjusted to be the same.

The server 1 compares the values of both graphs at corresponding time points in time series. The server 1 calculates and determines the degree of similarity between the user graph and the reference graph as follows. For example, the server 1 takes a differential value at each time point as indicated by arrows in (d). The server 1 takes, as the degree of similarity, a sum of the differential values at each time point in the target period. The server 1 compares this sum value, which is the degree of similarity, with a predetermined threshold value relating to the degree of similarity. The threshold value is a set value or the like of the processing definition information 58 in FIG. 1. For example, the server 1 determines that the degree of similarity is high when the sum value, which is the degree of similarity, is the threshold value or less, and determines that the degree of similarity is low when the sum value is greater than the threshold value.

The server 1 displays, on the screen of the terminal 2 of the user, information based on the matching and determination results of the above graphs, that is, information indicating the proximity between the shape of the user graph and the shape of the reference graph, and the like. The output example is “the shape of the female hormone graph of your examination result is similar to the shape of the reference graph, and thus, the state is assumed to be relatively good” or the like.

Upon the above processing, the server 1 may perform the comparison by using the entire graphs or may perform the comparison by using a part of the graphs, for example, numerical groups in a specific period. Moreover, not only the comparison of the graphs in one menstrual cycle, but also the comparison of the graphs in a plurality of menstrual cycles may be performed for the determination including the variations of the shapes of the graphs. The server 1 may also compare graphs of a plurality of kinds of female hormones. For example, the determination such that, among the LH, FSH, E2 and P4, the graph of a specific female hormone is proximate to the shape of the reference graph, and the graphs of other female hormones are apart from the shapes of the reference graphs, may be made.

With the above graph matching function, the user can know the proximity, conformity, and the like of the shape of his or her graph when the user's graph is compared with the reference graph, and can easily recognize his or her health state. In addition, as the number of data records increases, the shape of the graph becomes clearer, and thus, the analysis results are enriched, so that the user's motivation to record the data can be also enhanced.

[Processing Definition Information (1)]

Next, an example of the processing definition information 58 concerned with each processing of the aforementioned tendency analyses, action extraction, disease risk warning, and the like will be described with reference to FIGS. 20 to 23. It is an example of analysis mainly concerned with female fertility. Hereinafter, the unique reference information is applied to the example, but the medical reference information can be also applied.

FIG. 20 is a table showing an example of the processing definition information 58 concerned with the tendency analyses of the body temperature and the menstruation, and the disease risk warning. The processing definition information 58 in FIG. 20 has, as items, row number indicated by #, type, input, processing, and output. Each row indicates individual processing logic and also includes reference information to be applied. The column of the type indicates a rough type and a classification for the explanation. The column of the input indicates information on elements to be input for the processing. The column of the processing indicates contents of the processing logic. The column of the output indicates a specification and an outline of the output message.

(#1) The first row shows a processing example for checking the states of the menstrual cycle and the like in the tendency analysis of the menstruation. The input is the menstrual phase (a1 in FIG. 13) of the menstruation data input by the user. In the processing, the menstrual cycle a2 is calculated from [a1 differential value], which is the difference between the starting date of the last menstrual phase a1 and the starting date of the current menstrual phase a1, and is set as the “output 1.” The “output 1” is the last and the current menstrual cycles a2 or the like.

Moreover, as the processing, diseases relevant to the menstrual cycle a2 are checked. Settings are “disease 1a”=“frequent menstruation” and “disease 1b”=“infrequent menstruation.” In the processing, the menstrual cycle a2 is compared with x days to y days, which is a unique reference range K1. In the processing, “output 1a” is set when the relation a2<x days is satisfied, “output 1b” is set when the relation a2>y days is satisfied, and “output 1c” is set when the relation x days≦a2≦y days is satisfied. The “output 1a” is the possibility of “disease 1a,” a warning, and the like. The “output 1b” is the possibility of “disease 1b,” a warning, and the like. The “output 1c” is “good menstrual cycle a2” and the like.

Moreover, the processing checks variations in the plurality of the past menstrual cycles a2, compares the variations with predetermined values, determines tendencies such as improvement or deterioration, and outputs the results. In a processing example, a differential value (|last a2−k1|) between the value of the last a2 and the reference value k1 (e.g., 28 days) corresponding to a2 is calculated, a differential value (|current a2−k1|) between the value of the current a2 and the value k1 is calculated, the differential values are compared, and the “output 1d” is set when the latter is smaller (|last a2−k1|>|current a2−k1|). The “output 1d” is “improvement of the menstrual cycle a2” and the like. An example of the output message is “the last menstrual cycle is XX days, the current cycle is XX days, and extended by XX days, approaching the reference (k1)” or the like.

Determination in the case of deterioration is also possible in the same way as the above processing. As for the variation of the duration of the menstruation period a1 and its diseases, the processing the same as above is possible. Moreover, the values of the above references are not always the same for all the users, and the individual differences of the users may be reflected using statistical values and the like of the past menstrual cycles a2 of each user.

(#2) The second row shows an example of the disease risk warning processing concerned with the variation of the menstrual cycle. The relation “disease 2”=“irregular cycle menstruation” is set. The input is a2. For example, in the processing, a differential value between the current and the last menstrual cycles a2 and a differential value between the last menstrual cycle and the menstrual cycle before last a2 are calculated, and these [a2 differential values] are compared with X days which is the reference value. When the relation [a2 differential values]≧X days is satisfied, the “output 2” is set. The “output 2” is a2, [a2 differential value], the possibility of the “disease 2,” and the like. In another processing example, the periodic stability of the menstrual cycle a2 may be evaluated referring to the amount of variation (e.g., a plurality of [a2 differential values]) in a plurality of consecutive menstrual cycles a2.

(#3) The third row shows an example of the disease risk warning concerned with the variation of the menstrual cycle. The relation “disease 3”=“amenorrhea” is set. The input is a1. In the processing, the “output 3” is set when the relation [a differential number of days between the starting date of the last a1 and the current date]≧Y days is satisfied. The “output 3” is [the differential number of days between the starting date of the last a1 and the current date], the possibility of the “disease 3,” consultation recommendation, and the like.

Although not illustrated, the prediction of the next menstruation date is as follows. The inputs are the last menstrual phase a1 and the last menstrual cycle a2. The processing is [next predicted menstruation date]=[last menstrual phase a1]+[last menstrual cycle a2]. The output is [next predicted menstruation date].

Although not illustrated, the prediction of the next ovulation date is as follows. The input is the body temperature data. The processing is [next predicted ovulation date a3]=[minimum body temperature date] OR [body temperature falling date] OR [ . . . ]. OR is a logical sum and may be any one of [minimum body temperature date] and the like. The [minimum body temperature date] and the like are dates based on the medical definitions and indicate, for example, the date when the body temperature value becomes the minimum value. The output is [next predicted ovulation date a3].

(#4) The fourth row is an example relating to the tendency analysis of the body temperature, particularly to the temperature difference ΔT (a4 in FIG. 13). The input is the temperature difference ΔT of the body temperature data. Note that the temperature difference ΔT is calculated, for example, as follows. The calculation is [temperature difference ΔT]=[maximum body temperature]−[minimum body temperature] in the most recent menstrual cycle a2. Another processing example is ΔT=[average value of body temperature in high temperature phase t2]−[average value of body temperature in low temperature phase t1] in the most recent menstrual cycle a2.

The present processing determines presence of absence of the corresponding two phase patterns and the possibility of “disease 4” from the state of the above temperature difference ΔT and outputs the results thereof. The relation “disease 4”=“corpus luteum insufficiency” is set. The processing sets “good, since the relation ΔT≧0.3 degree is satisfied” (two-phase pattern)” and the like as the “output 4a” when the relation ΔT≧0.3 degree is satisfied in the most recent menstrual cycle a2. When the relation ΔT<0.3 degree is satisfied, the processing sets, as the “output 4b,” “not good, since the relation ΔT<0.3 degree is satisfied,” the possibility of “disease 4,” warning, and the like. 0.3 degree (° C.) is a reference value.

(#5) The fifth row is an example of checking the “disease 4” relating to the number of days of the high temperature phase t2. The input is the body temperature that occurs from the last day of the most recent menstruation. In the processing, for example, a variable b5=[the number of days of the high temperature phase t2] =[the number of days of the body temperature not less than (the minimum body temperature+0.3 degree)] is calculated. In the processing, the variable b5 is compared with Z days, which is the reference, the “output 5a” is set when the relation b5>Z days is satisfied, and the “output 5a” is b5, “good,” and the like. When the relation b5≦Z days is satisfied, the “output 5b” is set and includes b5, the possibility of the “disease 5,” and the like.

Although not illustrated, as previously mentioned, in the further processing, the time series variation of the temperature difference ΔT is referred to, the variation is compared with the predetermined value, tendency such as improvement or deterioration is determined, and as a result, “ΔT improvement,” “ΔT deterioration,” or the like is output. Moreover, in the processing, the determination is made based on the combination of the results of the above tendency analyses of the menstruation, the body temperature, and the examination results. In the processing example, the state of the above menstrual cycle a2 and the state of the body temperature difference ΔT are detected, and the output is set to caution or the like when a2 is within the reference range and the relation ΔT<0.3 degree is satisfied. Alternatively, when a2 is out of the reference range and the relation ΔT≧0.3 degree is satisfied, the output is set to caution or the like. Furthermore, in the processing example, when a2 is within the reference range, the relation ΔT≧0.3 degree is satisfied, and the value of a specific female hormone A is out of the reference range, the output is set to caution, “there is a possibility of abnormality in secretion of the female hormone A,” and the like.

[Processing Definition Information (2)]

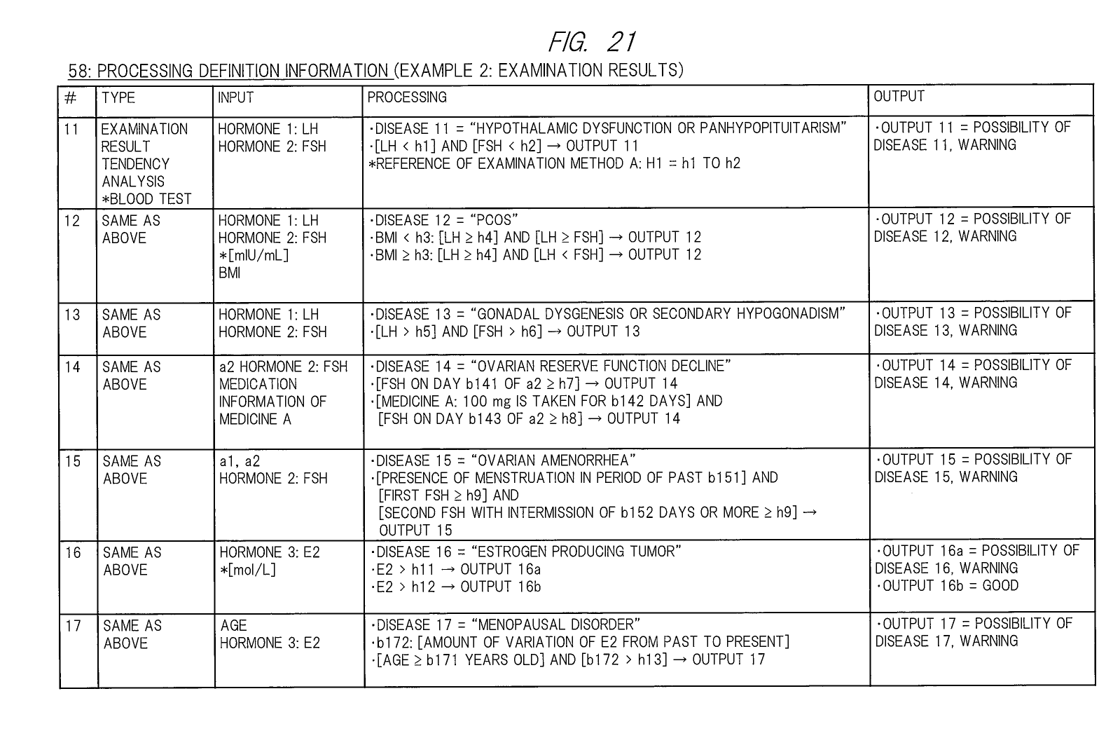

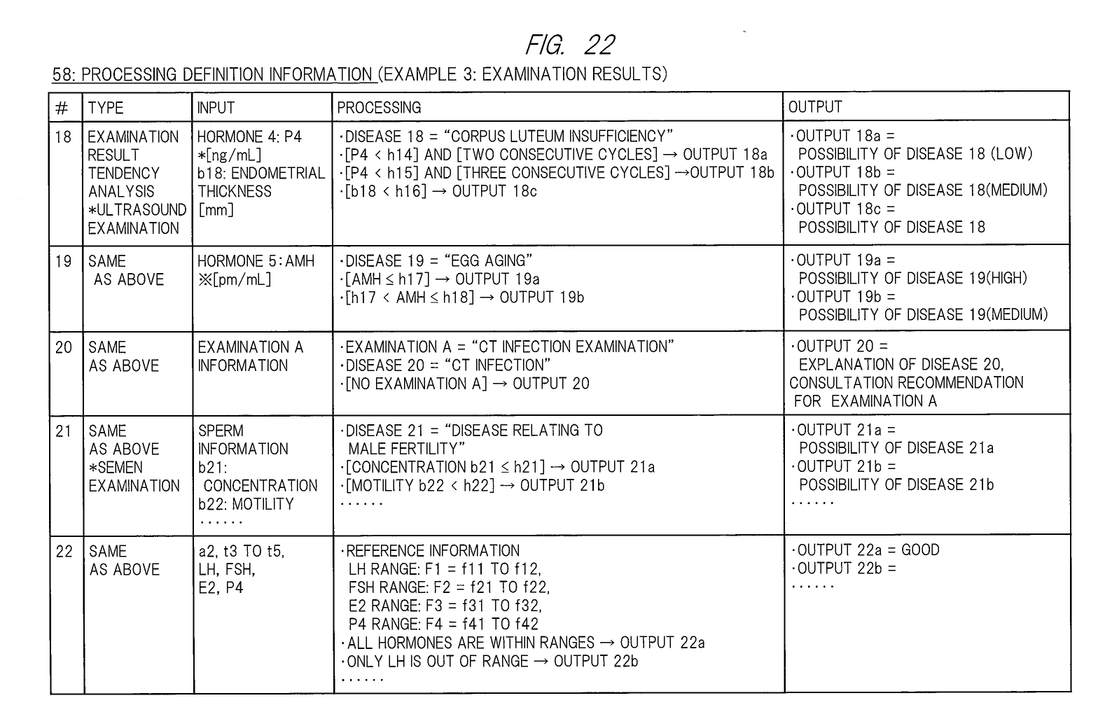

FIGS. 21 and 22 similarly show examples of the processing definition information 58 concerned with the tendency analyses of the examination results and the disease risk warning. These examples will be described with an example where the examination items are the plurality of types of aforementioned female hormones. The individual processing logic is defined according to the aforementioned differences in the examination methods and the like of the medical examination information 52, and the unique reference information according to the differences is applied. Even when the examination results are collected at the medical institution, the medical institution may be changed. Therefore, the examination results are set as an observation item for the user. This has a significance of deepening the understanding of the user.

(#11) The eleventh row shows an example relating to the checks of the LH, the FSH, and “disease 11.” The relation “disease 11” =“hypothalamic dysfunction or panhypopituitarism” is set. The inputs are the value of the examination item of the LH serving as the first female hormone and the value of the examination item of the FSH serving as the second female hormone. These are the values of the results of the blood test. In the processing, by using a range H1=h1 to h2 which is unique reference information for a specific examination method A and the like, the “output 11” is set when the relations [LH<h1] AND [FSH<h2] are satisfied, that is, when both the LH and the FSH are less than the reference values. The “output 11” is the possibility of the “disease 11,” warning, and the like. The present processing estimates the possibility of a specific disease with a combination of states of values of two female hormones and prompts confirmation of the causal daily life.

(#12) The twelfth row similarly shows an example of checking “disease 12.” The relation “disease 12”=“PCOS” is set. The inputs are the LH, the FSH, and the BMI. In addition, the units of the values of the LH and the FSH are defined as [mIU/mL] or the like. In the processing, by using the reference values h3 and h4, the “output 12” is set when the relations BMI<h3 and [LH≧h4] AND [LH≧FSH] are satisfied, and the “output 12” is similarly set when the relations BMI≧h3 and [LH≧h4] AND [LH<FSH] are satisfied. The “output 12” is the possibility of the “disease 12,” warning, and the like.

(#13) The thirteenth row similarly shows an example of checking “disease 13.” The relation “disease 13”=“gonadal dysgenesis or secondary hypogonadism” is set. The inputs are the LH and the FSH. In the processing, by using the reference values h5 and h6, the “output 13” is set when the relations [LH>h5] AND [FSH>h6] are satisfied, that is, when both the LH and the FSH exceed the reference values. The “output 13” is the possibility of the “disease 13,” warning, and the like.

(#14) The fourteenth row similarly shows an example of checking “disease 14.” The relation “disease 14”=“ovarian reserve function decline” is set. The inputs are the a2, the FSH, and the user input information on taking the medicine A. In the processing, by using the reference values h7, h8, b141, b142, and b143, the “output 14” is set when [FSH value on the b141 day of the menstrual cycle a2 is FSH h7]. Moreover, in the processing, the “output 14” is similarly set when [100 mg of the medicine A is taken for b142 days in the target period] AND [FSH value on the b143 day of the menstrual cycle a2 is FSH≧h8]. b141 and the like are the values for the number of days. The “output 14” is the possibility of the “disease 14,” warning, and the like. Like the present processing, judgment may be made by also taking the information on taking medicine into account in addition to the values of the female hormones.

(#15) The fifteenth row similarly shows an example of checking “disease 15.” The relation “disease 15”=“ovarian amenorrhea” is set. The inputs are the a1, the a2, and the FSH. In the processing, by using the reference values h9, b151, and b152, the “output 15” is set when [presence of menstruation in a period of past b151 days] AND [first FSH value is FSH≧h9] AND [second FSH value with an intermission of b152 days or more is FSH≧h9]. b151 and the like are the value for the number of days. The “output 15” is the possibility of the “disease 15,” warning, and the like.

(#16) The sixteenth row shows an example of checking the E2 and “disease 16.” The relation “disease 16”=“estrogen producing tumor” is set. The input is the value of the E2 serving as the third female hormone. The unit of the E2 is defined as [mol/L] or the like. When the units of the recorded examination results are different, the units are converted. In the processing, by using the reference values h11 and h12, the “output 16a” is set when the relation [E2>h11] is satisfied, and the “output 16b” is set when the relation [E2>h12] is satisfied. The “output 16a” is the possibility of the “disease 16,” warning, and the like. The “output 16b” is “good” (with ovarian function) and the like.

(#17) The seventeenth row shows an example of checking “disease 17.” The relation “disease 17”=“menopausal disorder” is set. The inputs are the age in the user attribute information 51 and the E2. In the processing, by using the reference values h13 and b171 and the variable b172, the “output 17” is set when the relations [age≧b171 years old] AND [b172>h13] are satisfied. b171 is the value for the age. The variable b172 is [the amount of variation of the E2 which is the difference between the most recent E2 value and the E2 value of the predetermined past]. The “output 17” is the possibility of the “disease 17,” warning, and the like. The present disease requires determination by personal history, and analysis of self-recording is required since there is a possibility that the user has changed the medical institution. Like the present processing, the determination may be made by also taking the attribute values of the user into account.

(#18) In FIG. 22, the eighteenth row shows an example of checking the P4 and “disease 18.” The relation “disease 18”=“corpus luteum insufficiency” is set. The input is the value of the P4 serving as the fourth female hormone. The unit of the P4 is defined as [ng/mL] or the like. In addition, for the input, information on the endometrial thickness (unit [mm]) of the uterus is used as the variable b18. The endometrial thickness is obtained from the result of a predetermined examination such as an ultrasound examination and is included in the examination result data 54 and the like of the user. In the processing, by using the reference values h14 and h15, the “output 18a” is set when the relation [P4<h14] AND the condition [the state has lasted for two consecutive cycles] are satisfied, and the “output 18b” is set when the relation [P4<h15] AND the condition [the state has lasted for three consecutive cycles] are satisfied. Moreover, the “output 18c” is set when the relation [b18<h16] is satisfied.

The “output 18a” is the presence of the possibility of the “disease 18,” the possibility thereof is low, and the like. The “output 18b” is the presence of the possibility of the “disease 18,” the possibility thereof is medium, and the like. The “output 18c” is the presence of the possibility of the “disease 18” and the like. In the present processing, the possibility of the disease is mildly estimated with a degree or level such as high/medium/low, and a warning and the like are output.

(#19) The nineteenth row shows an example of checking the AMH and “disease 19.” The relation “disease 19”=“egg aging” is set. The input is the value of the AMH serving as the fifth female hormone. In the processing, by using the reference values h17 and h18, the “output 19a” is set when the relation [AMH h17] is satisfied, and the “output 19b” is set when the relation [h17<AMH≦h18] is satisfied. The “output 19a” is the presence of the possibility of the “disease 19,” the possibility thereof is high, and the like. The “output 19b” is the presence of the possibility of the “disease 19,” the possibility thereof is medium, a warning, and the like.

(#20) The twentieth row shows am example of checking a specific examination and “disease 20.” The relation “disease 20”=“CT infection” is set. As the specific examination, examination A=“CT infection examination” is set. The input is information on presence or absence of the examination A based on the user input. In the processing, the “output 20” is set when the user did not undergo the examination A. The “output 20” is the explanation of the disease 20 and recommendation for taking the examination A. The determination may be made by also taking other information into account, such as the age in the user attribute information 51 and the dates of the examinations taken in the past.

(#21) The twenty-first row shows an example of checking the results of semen examination of a male user and is an example of particularly analyzing male fertility. “Disease 21” is set as “disease relating to male fertility.” The input is sperm information based on the results of the semen examination. For example, the concentration is the variable b21, the motility is the variable b22, and the like. In the processing, by using the reference values h21, h22, and the like, the “output 21a” is set when the relation [concentration b21≦h21] is satisfied. The “output 21a” is the possibility of the “disease 21a” (e.g., “oligospermia”). Moreover, in the processing, the “output 21b” is set when the relation [motility b22<h22] is satisfied. The “output 21b” is the possibility of the “disease 21b.” Similarly, in the processing, the possibilities of various relevant diseases, warnings, and the like are output by using variables of other sperm information. The output may include a list display of hospitals for male infertility, a suggestion for treatment such as artificial insemination, and explanation information thereof, depending on the determination results.

(#22) The twenty-second row shows an example of comprehensively determining the values of the plurality of types of female hormones. The inputs are the a2 and the values of the LH, the FSH, the E2, the P4, and the like. In the processing, as the unique reference information, a good numerical range or the like of each female hormone suitable for an examination method or the like is used. Examples of the numerical ranges include a range F1=f11 to f12 for the LH, a range F2=f21 to f22 for the FSH, a range F3=f31 to f32 for the E2, a range F4=f41 to f42 for the P4, and the like. As mentioned above, the numerical ranges of the reference may be numerical ranges for each period (e.g., t3 to t5) in the menstrual cycle a2, or the reference graphs may be used.

In the processing, the value of each female hormone in the menstrual cycle a2 in the target period is referred to, and the value is compared with the reference range of the corresponding type, and the state is determined. For example, when all the female hormones are within the ranges, the “output 22a” is set. The “output 22b” is set when only the LH is out of the range, and the “output 22c” is set when only the FSH is out of the range. When two of the LH and the FSH are out of the ranges, the “output 22d” is set. When predetermined three hormones are out of the ranges, the “output 22e” is set. When all the female hormones are out of the ranges, the “output 22f” is set. As described above, the health state of the user is determined with the combinations of the states of the plurality of female hormones, and the respective different outputs are determined.

[Processing Definition Information (3)]

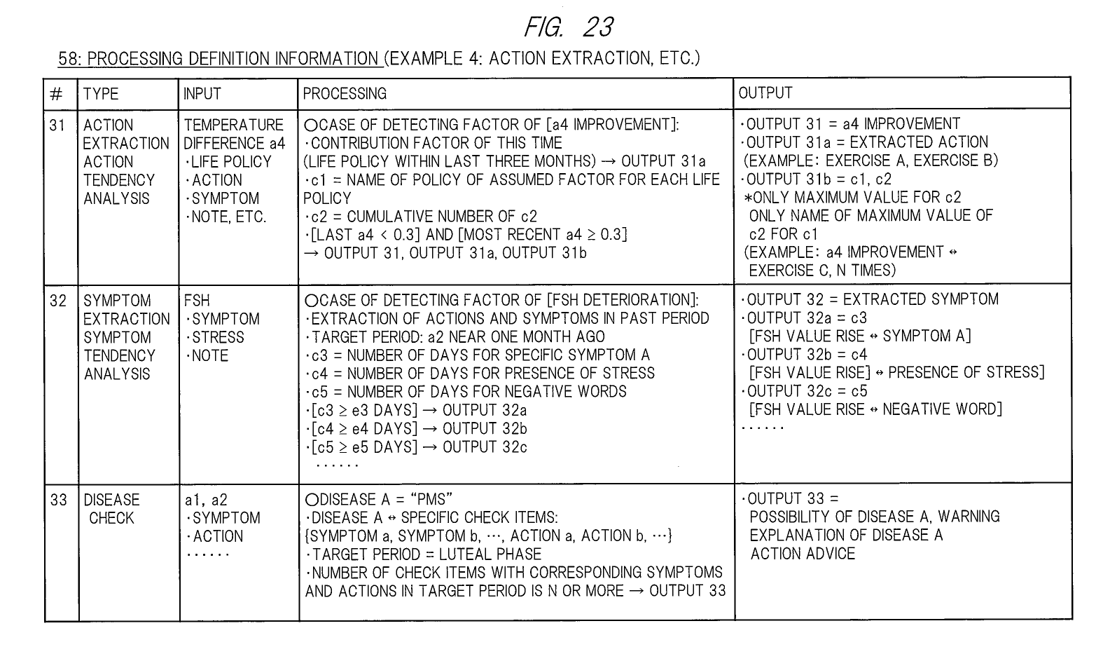

FIG. 23 shows an example of the processing definition information 58 on the analysis assistance to actions and action tendencies which may have causal relations with the body temperature and the examination results.

(#31) The thirty-first row is the determination of improvement or deterioration of the basal body temperature and extraction of actions assumed to contribute to the results. The actions to be extracted are actions that have been determined to contribute most to the results, including the actions in the most recent three months and the past. The inputs are information such as the temperature difference a4 in FIG. 13, and the life policy, actions, symptoms, notes, and the like in the user input information in FIG. 2. The life policy is stored in the user attribute information 51 in FIG. 2 and is a matter that the user has registered for particularly intensive implementation in exercise, diet, and the like in daily life.

A processing example of detecting a factor of improvement of the temperature difference a4 is as follows. In the processing, the “output 31,” the “output 31a,” and the “output 31b” are set when the temperature difference a4 in the last menstrual cycle was less than 0.3° C. and the temperature difference a4 in the most recent menstrual cycle became 0.3° C. or more. The most recent menstrual cycle is set to be the one within two months, and the last menstrual cycle is set to be the previous one of the most recent cycle.

The “output 31” includes determination information such as improvement or deterioration, for example, the improvement of a4. The “output 31a” includes information on the life policy within the last three months, which is a factor assumed to have contributed to the determination information of this time. The “output 31a” is, for example, the exercise A, the exercise B, or the like as an extraction action. The “output 31b” includes a name of the life policy most frequently determined to have contributed to the improvement or the deterioration including the past, and information on the cumulative number of the determinations of this life policy. The “output 31b” includes c1 and c2. c1 is the name of the policy of the assumed factor for each life policy such as exercise and diet. The policy of the assumed factor is a life policy assumed and determined to be a factor contributing to improvement or deterioration. c2 is the cumulative number of c1. In the “output 31b,” c2 includes only the maximum value, and c1 includes only the name of the maximum value of c2. For example, the “output 31b” is information such that the exercise C has been performed N times as a factor of the improvement of a4.

(#32) The thirty-second row is an example of performing symptom extraction and symptom tendency analysis. The inputs are, for example, FSH information of the tendency analysis results of the examination results, symptom and stress information by the symptom data input by the user, text information of the notes, and the like. Processing of detecting deterioration (value rise) of the FSH is as follows. In the processing, information on action and symptom in the past target period is extracted. For example, suppose that a symptom A, presence of stress, and the like are extracted. The symptom A is, for example, a headache, a depression, or the like. The “output 32” includes information on the extracted symptom and the like.

Moreover, variables for the processing are, in the target period, c3=the number of days in which the presence of specific symptom A is registered, c4=the number of days in which the presence of stress is registered, c5=the number of days in which negative words are registered in the text of the notes, and the like. These variables are defined for each processing logic. The values of these variables can be calculated based on the aforementioned user input information in FIG. 12 and the like. e3 to e5 and the like are used as unique reference information on these variables.

For c5, as a processing example, specific words such as “feeling bad” and “pain,” expressing feelings, symptoms, and the like of the user can be analyzed and extracted by text mining for calculation. c5 is not limited to the number of registered days, and the total number of appearances or the like may also be used.

For example, in the processing, the “output 32a” is set when the relation [c3≧e3 days] is satisfied, the “output 32b” is set when the relation [c4≧e4 days] is satisfied, and the “output 32c” is set when the relation [c5≧e5 days] is satisfied. The “output 32a” is c3, “symptom A associated with a rise in the FSH value,” and the like. The “output 32b” is c4, “presence of stress associated with a rise in the FSH value,” and the like. The “output 32c” is c5, “presence of negative words associated with a rise in the FSH value,” and the like. The message example is “the number of days with high stress in the last cycle was XX days,” “the number of negative notes in the last cycle was XX times,” or the like.

The above processing is an example of determining a not-good health state of the user, but processing of determining a good health state of the user is also possible. Other processing using information on the degree of the symptom such as severe/mild and on the degree of the stress such as high/low is also possible.

(#33) The thirty-third row is an example of checking disease

A=“PMS” as a specific disease. The input includes information such as the aforementioned a1, a2, actions, symptom, stress, and notes. Specific information items {symptom a, symptom b, . . . , action a, action b, . . . } are set as check items depending on the disease A. The check items are various symptoms, actions, and the like that are medically considered to be related to the disease A=PMS. The examples are symptom a=headache, action a=overeating, and the like. Moreover, a target period (e.g., luteal phase, three to ten days before the menstruation date, or the like) for referring to information of the check items is also set.

In the processing, information such as the symptoms and the actions of the user in the target period is referred to, the number of check items with corresponding symptoms and actions is calculated as an index value, and the “output 33” is set when the index value is N or more. For example, the index value is two when the user has the symptom a and the action a. The number N is a set value of each processing logic. The “output 33” is the presence of the possibility of the disease A and a warning, explanation information on the disease A, advice on actions in daily life appropriate for the disease A, and the like. The output example is “the possibility of the PMS is present. The P4 and the like in the luteal phase affect the PMS. For the symptom a, exercise b, diet b, and the like are recommended” or the like.

The index values of the check items and the set values of the reference may be definitions reflecting the number of days of each corresponding symptom and action, the degree of the symptom, the amount of the action, and the like. For example, the index value is five when the number of days in which the symptom a is registered is three and the number of days in which the action a is registered is two. For example, the index value is three even when the degree of the symptom a is at level 3 (severe) and the number of days registered is only one.

For the above processing, the results of the tendency analysis of each symptom or action may be used. For example, when frequency and continuity of the action a is high, the index value becomes high. Moreover, in the above processing, the determination of the possibility of the disease A is made with two values, presence or absence of the possibility, depending on whether the value is N or more. However, the determination may be made gradually with three or more values. Furthermore, in the above processing, in addition to the determination on the symptoms and the actions, the determination may be made by also taking information on the analysis results of the aforementioned body temperature, menstruation, examination results, and the like into account. For example, when deterioration of the menstrual cycle and deterioration of the values of the female hormones are observed in the same target period, the possibility of the disease A can be estimated to be higher.

In addition, in the above processing, the health state of the user, such as a disease, may be determined in consideration of ingestion of a specific food in the user's diet. The analysis unit 16 in FIG. 1 calculates, for example, as the index values, the number of days and the like of the ingestion of the specific food or nutrient such as a vitamin in the past target period. Then, the analysis unit 16 compares the index values with the reference values and determines the possibility of the disease A and the like.

Further, in the above processing, the health state of the user may be determined in consideration of the states of the user such as an amount of sleep, drinking alcohol, and smoking. Sleep and the like are provided as information items for user input. The analysis unit 16 in FIG. 1, for example, refers to the information on the sleeping hours in the target period to calculate the amount of the sleep, compares the amount of sleep with the reference value, and determines the health state of the user using the results.

[Relevant Information Search Function]

Next, the processing of the relevant information search function by the server 1 will be described. First, the server 1 determines and detects a tendency of the health state of the user, a possibility of a disease, and the like by the aforementioned functions of the tendency analysis, disease risk warning, and the like. Based on a keyword described in the processing logic of the above tendency analysis and the like and a keyword included in the output message of the analysis results, the server 1 automatically searches for relevant information on the Internet at the timing immediately after the detection. The server 1 acquires search result information with the keyword as a search condition. As the search results, public information on Web sites, which is closely relevant to the keyword and is, for example, medical relevant information or information such as a patient's diary, is acquired.

The server 1 stores index information such as the URL, which is the search result information, or the public information itself in the DB 50 as relevant search information. The server 1 displays the relevant search information on the screen of the terminal 2 of the user. A way of displaying is to, for example, display the relevant search information in a partial area of the screen of “MY medical record” of the user with a scroll or the like. The user can browse the information, scrolling in the area in the screen. When the information is a URL, a transition can be made into the Web page of the relevant search information. Another way of displaying may be to transition into the relevant search information by a link from a word in the output message to the user.

For example, when there is ‘PMS’ or ‘infertility’ as a keyword included in the analysis results of the health state of the user, the relevant search information on the keyword is displayed. With the relevant information search function, the user can easily browse the relevant information concerned with the health state of the user and the analysis results and can use the information as reference for treatment, action, and the like.

[Effects and the Like]

As described above, according to the health care system of the first embodiment, it is possible to achieve support for interpretation and acquisition of the health state including the body temperature and examination results of the user, and the medical information, enrichment and enhancement of providing information such as advice on the health state of the user and medical information, reduction in time and effort of the user for data input, securing motivation and willingness, and the like. By these, it is possible to care for the health state of the user and support treatment and examination. The same can be also applied to male users, not only to female users.

The present system provides functionally advanced service particularly for infertility treatment and the like. This enables the user to easily recognize the health state including his or her fertility and to obtain awareness of his or her health state. Therefore, the user can easily take actions such as treatment, examination, exercise, and diet to improve the health state and resolve the medical condition. The personal health data management enables the user to calmly recognize his or her health state without really considering the values of other people due to individual differences, specific reference values, and the like.

By specific information processing, the present system provides service which includes health data management, registration of actions, symptoms, and the like, and message output based on the advanced tendency analyses for each user. Thus, not only general advice, but also detailed useful information that conforms to the situation such as the health state of each user is provided, and judgement on the treatment and the like of the user is supported. For example, consultation recommendation, recommended actions, and the like relevant to the symptoms and actions of each user are provided as reference information. The user can easily recognize his or her health state including the treatment and the examination results, and this leads to self-awareness easily.

In general, the values and states of the body temperature, examination results, and the like of the user have individual differences and deviations, and variations are large along the time axis even with the same person. The present system provides personal health data management by time series and history of each information, tendency analysis of the variations and message output in consideration of the individual differences and the variations. The user can see his or her health state and the tendencies of the actions and the like, and this leads to self-awareness easily. From the results of the tendency analyses, even a slight improvement can be an encouragement or the like, and deterioration can be also a warning for future actions and the like.

The present system provides message information such the data of the graphs of the body temperature and the like, a warning of possibility of a disease, consultation recommendation at a hospital or the like, and action advice which are all relating to the health state of the user. The user can browse each data and information at any time and can utilize the output data. By using the output data, for example, the user can easily confirm his or her health state and ask the doctor about the health state upon medical examination, thereby preventing any omission of the confirmation and the like.

From the output message, the user can have a better understanding of the treatment by the medical institution and the examination results by the examination institution. Even when the user is concerned about the values of the examination results, the user can easily judge the values with reference to the output information. The user can easily judge what type of treatment, examination, and actions such as exercise and diet should be taken, and this easily leads to actual medical examinations and actions. The user is conscious of his or her disease and possibility of infertility and can easily take early countermeasures thereagainst.

The present system newly provides records and analyses of the examination results particularly of female hormones and the like. The user can easily understand the states of the female hormones and the like by the message of the tendency analyses of the examination results. The user can grasp his or her health state more in detail in a form combined with the body temperature, menstruation, and the like and can utilize for treatment and the like.

Also in outputting medical knowledge information, the present system does not uniformly output the information to all users equally, but provides the information at appropriate timing in relation to the health state of each user, such as the body temperature and the female hormones. Therefore, the user can also easily understand the medical knowledge information.

The present system provides information on action extraction and action tendency. From the information, the user can easily grasp whether his or her action is good or bad, its influence on the health state, the time series tendency, the actions likely to be effective, and the like. The user can easily recognize influences and results of his or her exercise, diet, and the like and can be easily motivated to take actions to improve his or her health state.

In the present system, not only mere input of data of values of body temperature and the like, but also various information such as actions, symptoms, and notes of feelings can be registered together in association with the data. These pieces of registration information are reflected on the screen of the user, analysis, and output at any time, accumulated as history, and can be browsed even afterward. The present system enables registration of not only information given by the medical institution, but also the user's subjective information on symptoms, feelings, and the like, and can analyze the user's subjective state from the information and provide the message appropriate for the state.

The present system provides comprehensive analysis using a combination of a plurality of elements such as body temperature, menstruation, examination results, symptoms, and actions, a disease risk warning, and the like. For example, the health state is determined by taking all of the body temperature, menstruation, examination results, symptoms, and the like into consideration. Measures for medical conditions with large individual differences, such as PMS, can be effectively supported. Compared with a conventional analysis technique using data of a single type such as body temperature, more advanced analysis and message output are possible.

The present system provides a mechanism which includes means for inputting each data from the terminal 2 of the user to the server 1 and the aforementioned input assistance function and reduces the burden for easy data input. Therefore, the user can save time and effort for each data input and be easily motivated for continuous data registration.

For menstruation management and infertility treatment, it is effective to continuously and accurately input and record data such as body temperature in time series. As the data input by the user becomes larger and more accurate, quality of the analysis by the present system becomes higher, the messages become more enriched, and the health state of the user can be grasped more accurately. For example, an accuracy of estimating possibility of a disease or the like becomes high. This makes the user easily motivated for daily continuous data input.

The present system continuously supports the user and alleviates the user's anxiety and distress during, before, and after the activities of the user, such as the treatment and the examination, at a clinical department including the obstetrics and gynecology department. For those in their 20s to 30s who have never gotten pregnant or who have not treated infertility, by utilizing the present service, even the user who is slightly anxious about his or her physical condition can be led to enlightenment, can recognize the risk and the like of unexpected diseases, can utilize the service for improvement of life habits, physical constitution, and self-awareness, and can prepare for future pregnancy and childbirth activities. Those in their 30s to 40s who have experienced infertility treatment and the like can be positively supported by using the present service, including estimation of causes of a disease and infertility and advice on specific countermeasures and treatment. Users who want to be deeply involved in the treatment can be also supported by functions relating to the examination result data. Users who are anxious about their personal medical condition can be also supported by providing individual messages.

The present system manages differences in medical institutions, examination methods, and the like and provides each user with support appropriate for his or her treatment and examination. Mistakes and confusions in comparison between values of different examination methods and the like can be also reduced. The present system uses the unique reference information to provide specific mild analysis, which comprehensively covers a plurality of medical institutions and a plurality of users, so that the system can support the user and medical care widely, not only coping with specific medical ideas and examination methods. The present system can be effectively applied to a field where medical ideas, examination methods, reference values, and the like are not medically standardized, as in the examples of infertility treatment and blood test. The present system can be applied without a premise of existence of population data, that is, data of specimens of a large number of people.

### Second Embodiment

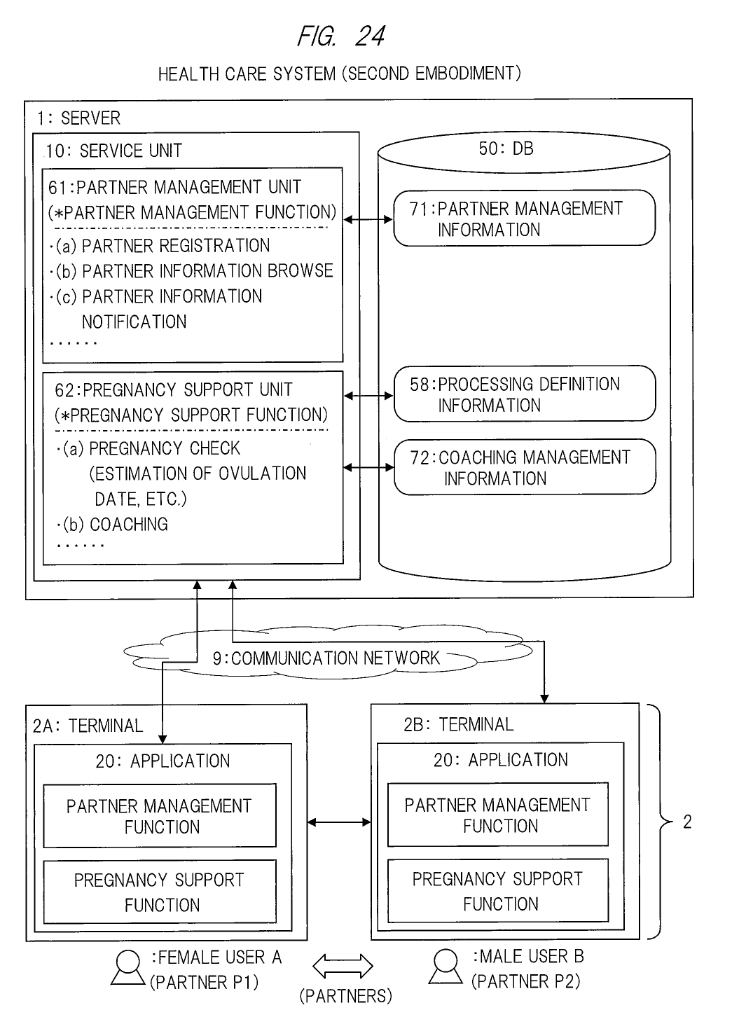

Next, a health care system according to a second embodiment will be described with reference to FIGS. 24 to 29. Hereinafter, different configurations in the second embodiment added to the first embodiment will be mainly described. Like the first embodiment, the second embodiment also targets the fields including obstetrics, gynecology, and reproductive medicine. Various types of health data can be recorded for each user, graphs and messages can be browsed, the health state of the user can be cared, and a self-awareness can be given.

Furthermore, the second embodiment provides service to support female natural pregnancy, childbirth, and the like. The main target users are those aged 16 to 49 years who are preparing for pregnancy and childbirth events. In particular, this service manages the relationship between partners of a female user and a male user and provides a function of supporting pregnancy activities in cooperation of both partners. The second embodiment provides the male and the female partners with a message, which is directly linked to the state of each individual user and which includes coaching, recommended actions, and the like for pregnancy activities, based on the user input data. This promotes the pregnancy activities of the male and the female partners and increases a success rate of pregnancy.

[System]

FIG. 24 shows an outline of the configuration of the health care system according to the second embodiment. As the terminal 2, a terminal 2A of a female user A and a terminal 2B of a male user B are connected to the server 1. The male user B is a partner P2 who is a husband, a lover, or the like of the female user A, and conversely, the female user A is a partner P1 as seen from the male user B.

The second embodiment has a partner management function and a pregnancy support function in the server 1 and the application 20. The service unit 10 of the server 1 includes a partner management unit 61 and a pregnancy support unit 62. The partner management unit 61 constitutes the partner management function and manages partner management information 71 in the DB 50. The pregnancy support unit 62 constitutes the pregnancy support function and manages coaching management information 72 and the like in the DB 50.

Each of the terminal 2A and the terminal 2B includes the application 20 as in the first embodiment. The application 20 implements a part of the partner management function and the pregnancy support function cooperating with the partner management unit 61 and the pregnancy support unit 62 of the service unit 10. Each of the terminal 2A and the terminal 2B accesses the service unit 10 of the server 1 from the application 20 and uses each function. The terminal 2A and the terminal 2B may communicate with each other as necessary to refer to the information on the other, for example, by using the partner management function.

[Partner Management Function]

The partner management function will be described. The partner management function includes functions of sharing information and managing cooperation between the male and the female partners. The partner management unit 61 includes (a) partner registration, (b) partner information browse, and (c) partner information notification as more detailed functions and processing units.

(a) The function of partner registration enables registration of partners and a couple by the female user A or the male user B. For example, the female user A can register the male user B as the partner P2, the female user A as the partner P1, and both as a couple. The server 1 receives a request for the above registration of the partners based on the user setting operation through the application 20 from the terminal 2A of the female user A or the terminal 2B of the male user B. Upon the reception of the request, the partner management unit 61 sets the female user A as the partner P1 and the male user B as the partner P2 in the partner management information 71.

As an example of the partner management information 71, a “partner” item may be provided in the user attribute information 51, and, for example, information such as a user ID of the partner may be stored in the item. The server 1 identifies and associates each data and the information on the user of the partner by the user ID of the partner.

(b) The function of the partner information browse is to perform processing for control in such a way that the female user A can browse various information on the male user B, who is her partner P2, on the screen of her terminal 2A. Likewise, with this function, the male user B can browse various information on the female user A, who is his partner P1, on the screen of his terminal 2B. Each user can switch between his or her information and partner's information for browsing and can also browse both information in parallel.

Based on the partner registration, the female user A can input and browse each information such as her health data on the screen of her terminal 2A as well as can input and browse each information such as the health data of the male user B who is the partner P2. Likewise, the male user B who is the partner P2 can input and browse each of his own information on the screen of his terminal 2B as well as can input and browse each information on the female user A who is the partner P1. For example, the user attribute information 51, the examination result data 54, the calendar input information 55, and the like of the male user B can be registered, and each graph, message, and the like can be browsed.

The partner management unit 61 may enable authorization settings for browsing and inputting the information of each other between the users of the partner, and the like. For example, the male user B may be authorized to be allowed to only browse the information on the female user A. It is also possible to set authorization for browsing and inputting in units of graphs, calendar, predetermined information items, and the like. Accordingly, it is possible to share and organize in a way that the female user A inputs a certain item, the male user B inputs a certain item, both input a certain item, and the like.

(c) The function of the partner information notification is to perform processing for automatically notifying the terminal 2 of the user of the other partner of the information in the predetermined items of the user of one partner and for causing the terminal 2 to display the information. The function of the partner information notification includes a watching function described later. The notification and watching items can be set by the user. For example, the male user B sets a specific item among the registered information on the female user A, who is the partner P1, as a notification and watching item. This causes the server 1 to automatically notify the terminal 2B of the male user B of the information in the specific item set. The male user B can always instantly browse the information in the notified item of his partner on the screen of the application 20 of his terminal 2B.

[Pregnancy Support Function]

The pregnancy support function will be described. The pregnancy support unit 62 includes (a) pregnancy check, (b) coaching of pregnancy activity, and the like as more detailed functions and processing units. The function of the pregnancy check in (a) includes functions of estimating ovulation date and the like. The pregnancy support function supports female pregnancy and pregnancy activities. The pregnancy support function not only supports individual female users, but also supports pregnancy activities in cooperation between the partners, the female user A and the male user B. The pregnancy support function is involved in and supports the activities of the male user B and acts on the male user B to prompt the pregnancy activities with the female user A.

States and results of female pregnancy, infertility, and the like are related not only to the health state and activeness including female fertility, but also to the health state and activeness including the fertility of the male partner. Thereupon, with the partner management function and the pregnancy support function, the second embodiment cares for the health states of the male and the female partners and supports pregnancy activities including treatment and actions of both. This makes the health states of the male and the female partners good and the fertility and activeness be in high states, thereby making the possibility of establishing pregnancy high.

[Pregnancy and Infertility]

Premised knowledge for pregnancy and infertility will be simply described. As for a healthy woman, the follicle grows in the follicular phase t3 and the low temperature phase t1 after the menstrual phase a1 in FIG. 13, and ovulation takes place based on stimulation by the female hormone in the ovulatory phase t4. In the luteal phase t5 and the high temperature phase t2, an egg waits for sperms in a fallopian tube. It is said that the life of the egg is about one day and the life of the sperm is about five days. Sperms advance in the uterus to the fallopian tube. When the sperm fertilizes the egg, the egg is implanted in the endometrium, and the pregnancy is established. When the pregnancy is established, there is no next menstruation, and the high temperature phase t2 often lasts in many cases. The above states concerned with physiology of the menstruation, ovulation, and the like and with fertility are correlated with the states of the body temperature, female hormones, and the like.

When natural pregnancy is desired and pregnancy activities are performed, it is necessary to maintain and improve the male and the female health states, including actions and the like in daily life for easy pregnancy. The present system cares for the health states of both male and female partners, coaches pregnancy activities, and comprehensively increases the possibility of pregnancy.

Meanwhile, as for infertility, infertility is diagnosed, for example, when, although sexual intercourse has been performed on a day near the ovulation day, a non-pregnant state lasts for more than a predetermined period. Infertility may be caused by both men and women. The present system analyzes the causes of the infertility and the like for both male and female partners to provide advice, thereby comprehensively increasing the possibility of pregnancy.

Incidentally, an example of artificial insemination (AIH) for that regard is as follows. The artificial insemination is conducted on a day near the ovulation day. Male sperms are collected, carefully selected, and injected into the female uterus. This supports fertilization for natural pregnancy. Thereafter, the establishment of pregnancy is determined by examination and the like. An example of in-vitro fertilization (IVF-ET) is as follows. In the in-vitro fertilization, eggs taken out from female ovaries by an ovulation inducing agent are fertilized with sperms collected from a man and cultured in an incubator, and a fertilized egg (embryo) is transplanted into the female uterus. Thereafter, the implantation is supported by prescription of medicine and the like. Thereafter, the establishment of pregnancy is determined by examination and the like.

[Pregnancy Check (1)]

A processing example of the function of the pregnancy check in (a) of the pregnancy support function will be described. The pregnancy support unit 62 uses the user input data to perform processing of estimating the ovulation date greatly related to the establishment of pregnancy based on the processing definition information 58. Note that this estimated ovulation date is different from the aforementioned predicted ovulation date a3 and is an ovulation date by more detailed comprehensive estimation.

The server 1 first acquires or calculates information on elements such as (a) to (h) below from the user input data and the like. (a) Information on presence or absence of ovulation and the like, which are the result of the ovulation test. This is the information on the result of the ovulation test which is performed by the user herself using an ovulation checker or the like or the information on the result of a predetermined examination conducted at a medical institution or an examination institution. The ovulation checker is a test drug for detecting, for example, the concentration of the LH contained in urine or blood and presents a high value, that is, positive, just before the ovulation date.

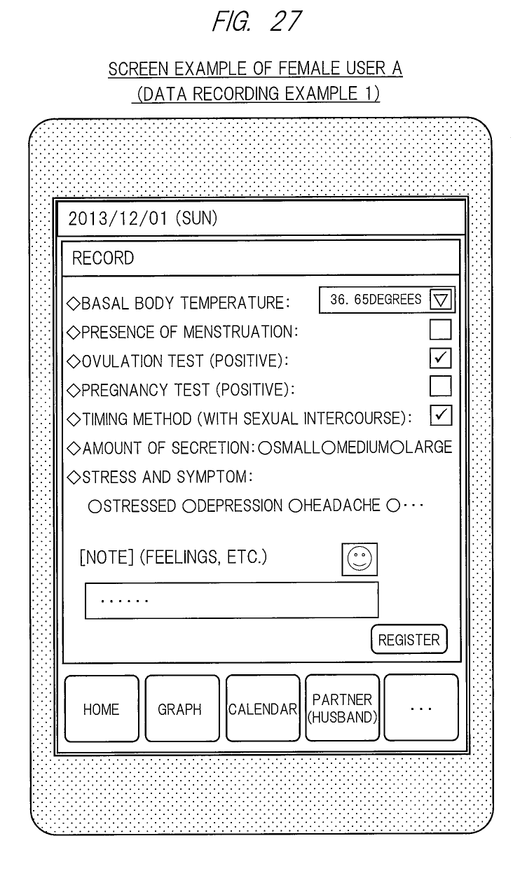

(b) Predicted ovulation date a3. This is an ovulation date predicted from the aforementioned menstrual phase a1 and the menstrual cycle a2. (c) Information on presence or absence of sexual intercourse by the timing method on a day near the ovulation day. (d) Information on the state of the menstruation, cervical mucus, and the like input by the user. For the information (a), (c), and (d), input values on the screen as shown in FIG. 27 described later can be used.

(e) Information on age, BMI, and the like based on the user attribute information 51. (f) Information on a degree of stress or a degree of a specific symptom. (g) Information on an amount and the like of a specific exercise. (h) Information on the amount and the like of a specific diet. (f) to (h) are obtained from the symptom data and action data input by the user and the analysis results thereof.

The server 1 estimates the ovulation date by using the information on the elements as in (a) to (h) above. Calculating this estimation is possible in many ways, not limited to one. First, simply, the ovulation date by the ovulation test of (a) may be used as it is, or the predicted ovulation date a3 of (b) may be used as it is. The server 1 may also estimate the next ovulation date by also taking the information of (d) to (h) into account. The server 1 may also accumulate information on the results of estimating the ovulation date of the user in time series, calculate deviation between the estimated ovulation date and the ovulation date derived from the results of the ovulation test, and take into account the deviation for reflection and feedback for the subsequent estimations. The server 1 may also update the calculating formula of the above estimation by administrator setting or automatic modification in consideration of the estimation results and the accuracy thereof. The server 1 may estimate the above ovulation date by also taking values of various female hormones into account.

[Pregnancy Check (2)]

Other processing examples of the function of pregnancy check in (a) of the pregnancy support function are as follows. Based on the processing definition information 58 and the results of the tendency analyses and the like in the first embodiment, the pregnancy support unit 62 may determine the state of ease of natural pregnancy, possibility of establishment of pregnancy, possibility of infertility, and the like.

Since presence or absence, possibility, and the like of establishment of pregnancy can be determined by an existing pregnancy test and a predetermined examination, the server 1 first uses the information on the result of the pregnancy test when the information has been input by the user. The pregnancy test is a test or examination conducted with a pregnancy test drug or the like by the user or a medical institution and the like. The pregnancy test drug detects, for example, a concentration and the like of human chorionic gonadotropin (hCG) which is a female hormone contained in urine or blood. A positive result of the pregnancy test indicates that the pregnancy is established or the possibility thereof is high.

The server 1 may estimate a degree of the possibility of establishment of pregnancy by using the body temperature and menstruation input by the user and the values of the female hormones such as the LH. In the processing, the possibility of establishment of pregnancy is estimated to be high, for example, when the state without menstruation, the state of [ΔT≧0.3 degree], and the state where the values of the female hormones such as the LH fall within the unique numerical ranges lasts for a predetermined number of days or longer.

A processing example of determining the possibility of infertility is as follows. The server 1 judges the health state including tendencies of the body temperature in each phase, the temperature difference ΔT, the menstrual phase a1, the menstrual cycle a2, the predicted ovulation date a3, the number of days of each phase, the values of the female hormones such as the LH, and the like in the time series user input data. The server 1 confirms presence or absence of sexual intercourse on a day near the ovulation day and confirms the results of the ovulation test and the pregnancy test. The server 1 determines that there is a possibility of “infertility” when pregnancy is not successful despite the presence of sexual intercourse around the ovulation date over a predetermined period, and outputs a corresponding message. The output example is “the possibility of infertility is estimated from the menstruation and the values of the female hormones. We recommend you to take a medical examination if you are concerned. For example, a medical institution A and the like provide treatment A and examination A for infertility” or the like. Note that, in the first and second embodiments, the possibilities of various diseases including not only infertility, but also diseases belonging to other clinical fields may be checked, and countermeasures thereagainst may be promoted. This can increase the possibility of pregnancy.

[Pregnancy Check (3)]

As one of the pregnancy checks, a processing example of determining the ease of natural pregnancy is as follows. The partner management function and the pregnancy support function analyze and grasp the states including the tendencies of the actions, symptoms, and the like of each of the male and female users, which affect the ease of pregnancy, as in the first embodiment. The pregnancy support function calculates an index value representing the state of ease of pregnancy or an index value representing fertility based on the grasp of the health states of the male and the female users. The index values are helpful information for guide. The pregnancy support function provides a message including the above states and index values of the male and the female users and also provides a message such as recommended actions, so that the man and the woman are led to successful pregnancy.

The pregnancy support function may calculate the above index values by also taking the following action and symptom states into account. For example, excessive exercise, lack of exercise, overeating, dieting, irregular diet, unbalanced food, and the like are grasped as the tendencies of the actions of the female user A. High stress, specific symptoms, and the like of the female user A are also grasped. These states influence the body temperature, the menstruation, the states of the female hormones, and the like and influence the states of the uterus, ovaries, and the like which are concerned with fertility, that is, influence the ease of pregnancy. Similarly, the actions and the states concerned with fertility of the male user B are grasped. The pregnancy support function performs the calculation in such a way that the index values become low accordingly when the actions and the states of the symptoms are not good.

Moreover, the pregnancy support function may judge, for example, the periodic stability of the menstrual cycle a2 and the like in the target period in the time series registration data of the user and set the above index values to be high values when the stability is high. Furthermore, the pregnancy support function may calculate the above index values by also taking the age, the disease, the anamnesis, and the like in the user attribute information 51 into account. For example, when the age is old, the index value of the fertility is calculated to be low accordingly.

In addition, the pregnancy support function may determine the ease of pregnancy and the like from a combination of the health states of the above male and the female partners and provide the male and the female users with a message of the determination results. For example, by multiplying the values of the health state of the female user A by the values of the health state of the male user B, or the like, the index values in the unit of the couple are calculated. For example, when the male user B has relatively low fertility although the female user A has high fertility, the ease of pregnancy in the unit of the male and female couple is determined to be low. Then, as the output message, the notification includes the information stating the above and the index values. The message acts especially on the male user B by recommendation for action and treatment, and the like. The male and female users can be conscious of activeness of their pregnancy activities and the ease of pregnancy by looking at the above information.

[Coaching]

A processing example of the coaching function in (b) of the pregnancy support function is as follows. As the output message information for the male user B, the pregnancy support unit 62 provides information such as coaching, advice, and recommendation for activating the pregnancy activities with the female user A, as reference information. The coaching is, in other words, support or suggestion for achieving a goal of successful pregnancy or for increasing the possibility of pregnancy as much as possible. The function of coaching is to provide a message including coaching information for activating involvement and actions including communications and the like between the female user A and the male user B of the partners. This promotes the male and female pregnancy activities and increases the possibility of pregnancy. Examples of coaching are shown later.

As screen examples of the terminal 2 of the user in the second embodiment, the MY medical record screen, the calendar screen, the input field for one day, the input field of each information item, and the like in the first embodiment are similarly provided. Other screen examples are as follows.

[Female User Screen]

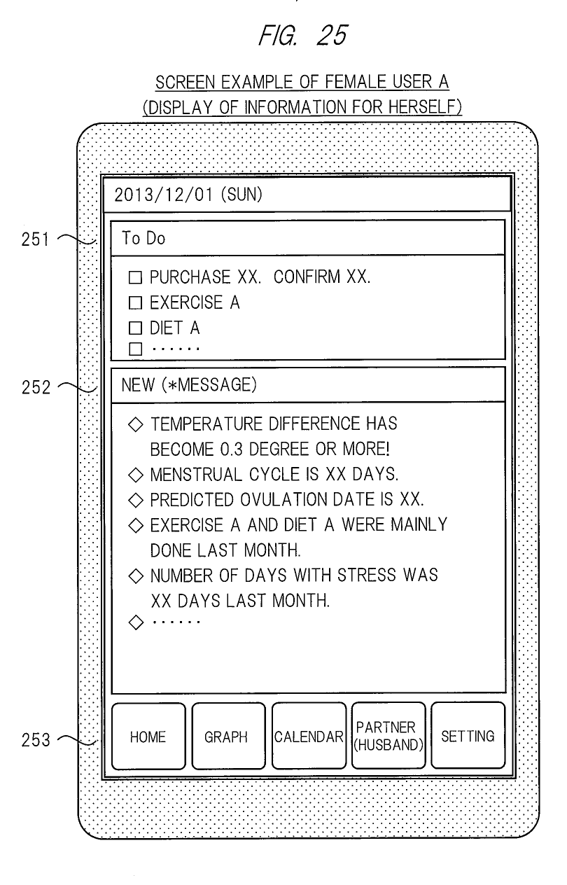

FIG. 25 shows a first screen example of the terminal 2A of the female user A. The terminal 2A is a smartphone or the like. The screen in FIG. 25 displays various information for one day in the calendar as a screen displaying the information of the female user A for herself. This screen has a “To Do” field 251, a “NEW” field 252, a menu 253, and the like.

The “To Do” field 251 displays “To Do” of the female user A for the day, that is, list information on what should be done. On another screen, for example, the aforementioned calendar screen, the female user A can select a date and register “To Do” information by text or choices. Then, the registered information is displayed in the “To Do” field 251. Examples of the “To Do” information include, for example, purchase of a test drug, plans of hospital visit and examination, and plans of actions including exercise, diet, and the like, which are freely applicable.

The “NEW” field 252 displays the latest output message information according to the health state of the user. The output messages include the aforementioned tendency analyses, action extraction, disease risk warning, and results of the pregnancy check and the like.

The menu 253 shows menu buttons for operations of selecting functions. For example, there are “HOME,” “graph,” “calendar,” “partner,” “setting,” and the like in the menu 253. With the “HOME” button, a transition can be made into the screen of the HOME of the service or into the screen such as the aforementioned “MY medical record.” With the “graph” button, a transition can be made into a screen displaying a graph such as the body temperature-menstruation graph. With the “calendar” button, a transition can be made into the calendar screen. With the “partner” button, a transition can be made into a screen displaying the partner information. With the “setting” button, a transition can be made into a screen for user settings of the partner registration and the like.

The user can switch the display between his or her information and the partner information with the “partner” button. For example, the female user A can make a transition into a screen displaying the information on the male user B, who is the partner P2, with the “partner (husband)” button. The screen of the partner information displays information with the same contents which the partner user browses. Moreover, to return to the screen of her information from the screen of the partner information, the female user A should simply press the “partner (me)” button again. The terms “(husband)” and “(me)” indicate information for distinguishing one from his or her partner.

When the “partner” button is pressed down, the application 20 requests a browse of the partner information to the server 1. Upon the reception of the request from the terminal 2A of the female user A, the server 1 reads out the requested information on the male user B of the partner P2 from the DB 50 and transmits the information to the terminal 2A of the female user A. This enables the female user A to browse the information on the male user B, who is the partner P2, on the screen of the partner information. The same processing is also performed when the sex is reversed. Note that the female user A can input the information on the male user B on behalf of the male user B, who is the partner, on the screen, and vice versa is also possible in the same way.

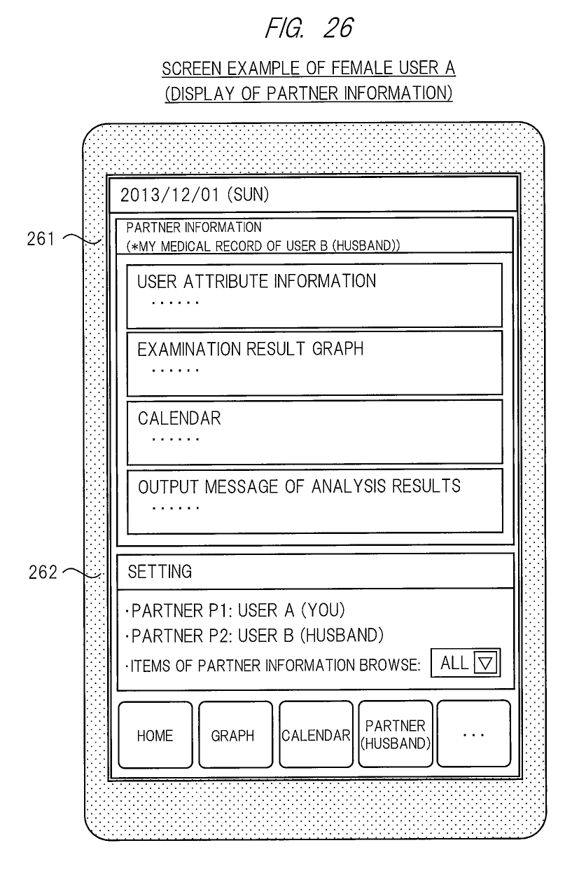

FIG. 26 shows a case of browsing the information on the male user B of the partner as a second screen example on the terminal 2A of the female user A, and a user setting example. This screen has a “partner information” field 261, a “setting” field 262, and the like.

The “partner information” field 261 displays, according to the transition requested from the “partner” button in FIG. 25, all or designated one piece of various information such as the user attribute information, the graphs, the calendar, and the output messages for the analysis results as the information in the aforementioned MY medical record of the male user B who is the partner P2.

The “setting” field 262 also shows an example of displaying the user setting information of the partner registration together. Normally, the information in the “partner information” field 261 is displayed on the entire screen of the terminal 2, and the information in the “setting” field 262 is separately displayed according to the “setting” button in FIG. 25. In the “setting” field 262, setting of a user to be registered as a partner, predetermined items to be browsed and input, and authorization setting are possible.

FIG. 27 shows a screen example of data recording for herself as a third screen example on the terminal 2A of the female user A. In this screen, as input information items for the day, there are basal body temperature, presence of menstruation, ovulation test, pregnancy test, timing method (sexual intercourse), an amount of secretion, stress and symptoms, note, and the like. In the ovulation test item, positive or negative of the result of the ovulation test can be input. In the pregnancy test item, positive or negative of the result of the pregnancy test can be input. In the timing method (sexual intercourse) item, presence or absence of sexual intercourse can be input. In the stress and symptom item, presence or absence, a degree, and the like of the stress and symptom can be input. In the note item, arbitrary text indicating feelings, memos, and the like can be input, and the feelings and the like can be input by selecting a face mark.

[Male User Screen]

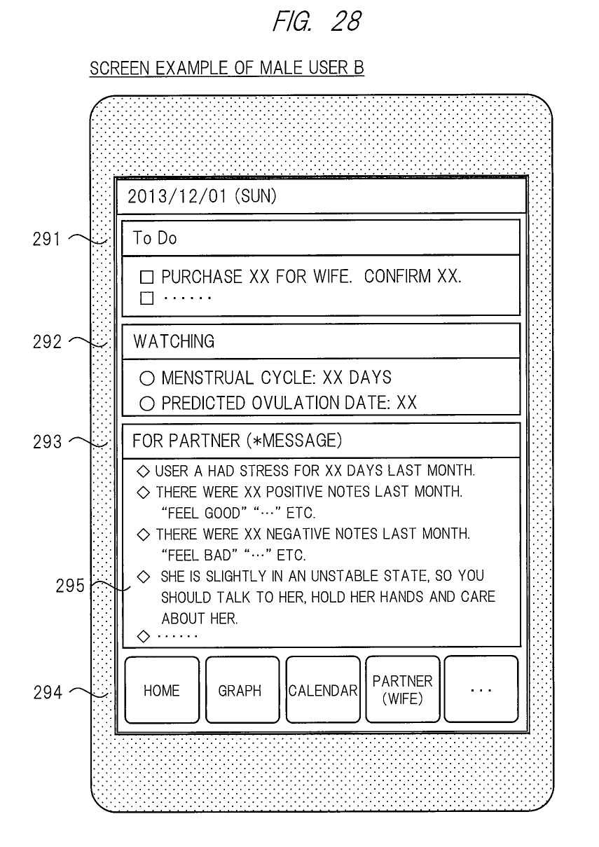

FIG. 28 shows a screen example on the terminal 2B of the male user B who is the partner P2 of the female user A. This screen is a screen example of the male user B browsing mainly his information and includes therein a part displaying the partner information. This screen includes a “To Do” field 291, a “watching” field 292, a “for partner” field 293, a menu 294, and the like.

The “To Do” field 291 displays the “To Do” information for the male user B himself in the same way as the case of the female user A. The male user B can confirm his “To Do” information in the “To Do” field 291. With the “To Do” information, each user can streamline activities such as pregnancy activities, treatment, and examination. Note that the “To Do” field 291 may automatically display the same contents of the information in the “To Do” field 251 of the female user A who is the partner P1. Moreover, an item for displaying the information in the “To Do” field 251 of the female user A, who is the partner P1, may be separately provided.

Among the information on the female user A of the partner P1, the “watching” field 292 displays particularly information in a predetermined item set by the user as a watching item. The information in the watching item is displayed by the function of the partner information notification. For example, the female user A or the male user B sets the menstrual cycle, predicted ovulation date, and the like of the female user A as the information in the watching item set by the user. In this case, the values of the latest menstrual cycle a2, predicted ovulation date a3, and the like of the female user A are automatically displayed in the “watching” field 292. With the information in the “watching” field 292, the male user B can always instantly confirm the information in the watching item relating to the female user A of the partner P1 on his terminal 2B. That is, it is easy to check the health state and the like of the partner. Each user can set an item with the information he or she cares about as the watching item. The same applies when the watching item is provided on the screen of the female user A.

The “for partner” field 293 is a field for displaying output message information for the male user B and includes particularly display of the information for the female user A of partner P1. The partner management unit 61 and the pregnancy support unit 62 generate a message including coaching information to be displayed in this field 293. Note that, on the screen of the female user A in FIG. 25, a “for partner” field may also be provided to display coaching information and the like for the female user A, in the same way as above. The above “watching” and “for partner” fields may be integrated, and the “coaching” field may be separately provided.

The output messages in the field 293 include messages of the results of the tendency analysis of the health state, action extraction, disease risk warning, and the like of the female user A of the partner P1. For example, suppose, as the health state of the female user A, there are many days with stress last month and in the last menstrual cycle based on the results of the extraction and the analyses of the past symptoms. The pregnancy support function displays a message conveying the health state of the female user A in this field 293. A message example is “the user A had stress for XX days last month” or the like. Another example displays a message of the analysis result of the text of the registered notes of the female user A. For example, a message conveys a positive word and a negative word, the number of registered days, and the frequency thereof, and the like.

The output message in the field 293 also includes display of the coaching information on the pregnancy activities with the female user A of the partner, as exemplified particularly in 295. 295 is an example of the message such as advice and recommendation for actions of the male user B to act on the female user A, as the coaching information.

The coaching function uses the coaching management information 72 to generate and determine the coaching information for activating the pregnancy activities based on the data of the registration of the female user A, analysis results, and the like. In the coaching management information 72, processing contents for generating and providing the above coaching information and information such as specific actions are set. For example, the health state of the female user and information such as advice on actions which are candidates for the output coaching information are set in association with each other.

The pregnancy support function determines the health state of the female user A, for example, states of the specific symptoms, the degree of stress, the number of negative words, and the like. The pregnancy support function may further determine whether the health state of the user is good or bad and how much degree of stability of the health state of the user is, from the determined state of the symptom and the like. Examples include “stable state,” “slightly unstable state,” “unstable state,” and the like. Then, in consideration of the above health state of the female user A, the coaching function determines, based on the coaching management information 72, the coaching information such as advice on action and recommendation for the male user B to act on the female user A according to the state. The coaching information includes suggestions of specific actions to act on, for example, caring, confirming, speaking, holding hands, and the like. The coaching information may also include the state of the female user A, which is the reason for the suggestion, for example, “high stress,” “slightly unstable state,” and the like.

Other coaching may include consultation recommendation for treatment, examination, and like appropriate for the health state of the user as previously mentioned. Other coaching may provide advice on actions and the like depending on the health state including the fertility of the user. For example, when the examination results indicate that the state of the ovaries or the sperm is not good, advice on exercise, diet, and the like considered to be effective for improvement may be provided, or actions to be suppressed among the actions registered by the user may be suggested. Other coaching may include recommendation information on actions that can be performed together by men and women, for example, entertainment, events, and the like. This promotes communication and the like between the partners.

Moreover, the coaching function compares the health states of the male and the female partners and decides the coaching information. For example, when one of a man and a woman is in a good state and the other is not in a good state, the coaching information with contents saying that the user in the good state should care about the user not in the good state is output. Furthermore, information appropriate for each case of when both man and woman are in good states, when both man and woman are not in good states, and the like is output.

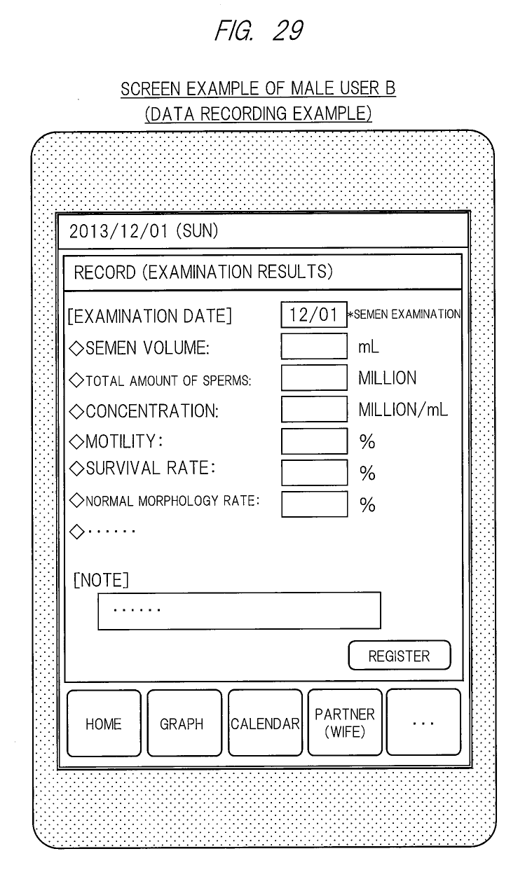

FIG. 29 shows an example of inputting the information on the aforementioned examination results in data recording as the second screen example on the terminal 2B of the male user B. In the case of aiming at natural pregnancy and making self-help effort for a certain period of time, the man is supposed to have no problems in his fertility. Therefore, examination of the male fertility is desired to be performed at least once, and this fact is the premise of using the second embodiment. Information on the examination results is information that is greatly concerned with the male fertility. This screen includes, as items, examination date, semen volume, total amount of sperms, concentration, motility, survival rate, normal morphology rate, note, and the like. Similar to the screen of the female user, the screen of the male user may be provided with additional input fields of various symptoms, stress, and the like concerned with the health state. Moreover, as for the examination items, history thereof can be referred to in time series with a graph.

[Effects and the Like]

The effects of the second embodiment are as follows. With the functions of the first embodiment, each user of the male and female partners can first recognize his or her own health state by referring to the graphs and the messages and can record and schedule his or her actions and the like. Then, with the functions of the second embodiment, each male or female user registered as the partner can easily and mutually share, browse, and input the information between the partners. The partners can mutually refer to and confirm each other's health state, actions, feelings, and the like. Each of the male and the female users can share a plan and a schedule of actions and the like with the partners by looking at the calendar, “To Do,” and the partner information. The male and the female users can work on pregnancy activities in cooperation and harmony while matching wills, schedules, and the like. It is easy to do the pregnancy activities while doing jobs or the like. By watching the partner information, mutual understanding and communication between a man and a woman progress, and understanding each other's feelings becomes easy.

Based on tendency analyses and the like, the present system grasps the health states of men and women including the following relevance and provides advice and the like on pregnancy activities, so that possibility of pregnancy can be increased. Medically, actions such as the user's life habits are linked to states of elements such as body temperature, menstruation, sperms, and female hormones, states of symptoms and stress, male and female fertilities, a state of ease of pregnancy, possibility of pregnancy or infertility, states of possibility and the like of disease, and the like. In particular, it is said that action tendencies and a state of tendency of variation including periodic stability in time series values of each element are greatly related to fertility, pregnancy, and possibility of disease.

For example, actions such as inappropriate exercise and diet, stress from work, stress in relation to the partner in the female user A lead to uneven and unstable body temperature difference, menstrual cycle, female hormones, and the like, and these may appear as symptoms such as so-called menstrual disorder, menstrual pain and depression, unstable feelings, and the like. Moreover, risk of a specific disease also increases depending on the degrees. Similarly in the case of the male user B, poor states of actions, stress, and the like affect the states of reduction and the like in sperms and male hormones in the examination results, that is, lead to a disease such as oligospermia, reduction in fertility, and cause of infertility.

Conventionally, there has been no service relating to pregnancy for men, and there has been no service to support pregnancy activities of male and female partners. The present system involves not only particularly women but also men to support and coach. By supporting the pregnancy activities of the partners, a success rate of natural pregnancy and the like can be increased compared with the case of pregnancy activities of the woman alone. Also in the case of infertility treatment, activities in cooperation with the partners can be supported. The uneasy feelings toward the infertility treatment can be also shared between the man and the woman and alleviated. The health state can be also cared for in the same way as above during pregnancy and after childbirth, not only before pregnancy.

The present invention is not to be limited to the above embodiments and may be modified in various ways within a scope not deviating from the gist thereof. Another embodiment includes the following. The present system counts the amount of data input by the user from the application 20 of the terminal 2 of the user, the number of days of the data input, and the like and manages the numbers as index values. The server 1 stores the above index values and displays them on the screen of the application 20. According to the above index values, the present system may give the user benefits and the like on the service. This further motivates the user to input data.

### INDUSTRIAL APPLICABILITY

The present invention can be applied to the fields of medical care and health care including obstetrics, gynecology, and reproductive medicine.

### EXPLANATION OF REFERENCE CHARACTERS

1 . . . server, 2 . . . terminal, 3 . . . medical device, 4 . . . terminal, 9 . . . communication network, 10 . . . service unit, 11 . . . user attribute information registration unit, 12 . . . medical information setting unit, 13 . . . health data management unit, 14 . . . graph creation unit, 15 . . . calendar input unit, 16 . . . analysis unit, 17 . . . message output unit, 18 . . . auxiliary unit, 20 . . . application, 21 . . . body temperature-menstruation data input unit, 22 . . . examination result data input unit, 50 . . . DB, 51 . . . user attribute information, 52 . . . medical examination information, 53 . . . health data, 54 . . . examination result data, 55 . . . calendar input information, 56 . . . analysis information, 57 . . . output message information, 58 . . . processing definition information, 61 . . . partner management unit, 62 . . . pregnancy support unit.

1. A health care system, comprising:
a server device providing service for caring for a health state of each user; and
a terminal of the user,
wherein the server device includes:
a data management unit registering and managing health information including examination result data of each user, and user attribute information including information on a plurality of attributes of each user, including sex, age, a medical institution or an examination institution used, states of treatment and disease, and history, based on operation from the terminal of the user, and associating and managing, as medical examination information, each of a plurality of pieces of information including the medical institution, the treatment, the examination institution, examination, an examination method, and a numerical range of medical reference information;
an analysis unit determining, by using the user attribute information of the user and the medical examination information, the health state of each user, including a tendency of variation of values of an examination item and including good or bad of the values of the examination item and relative improvement or deterioration in time series of the values of the examination item, based on comparison result between time series values of the examination item of the examination result data of the user and the numerical range of medical reference information corresponding to the examination item and the medical institution or the examination institution used by the user and associated with the examination and the examination method of the medical institution or the examination institution, and determining the good state when the value of the examination item is within the numerical range of the medical reference information, and determining the improved state when the value of the examination item varies and approaches the numerical range of the medical reference information; and
an output unit outputting, to the terminal of the user, information including a time series graph of the examination result data and a message appropriate for the health state of each user, and
wherein the medical examination information includes setting of the numerical range of the medical reference information which is different according to the medical institution, the treatment, the examination institution, the examination, or the examination method.

2. The health care system according to claim 1,
wherein the data management unit registers and manages the health information including body temperature and menstruation data of each user based on the operation from the terminal of the user,
the analysis unit determines the health state of each user, including tendencies of variations of values of body temperature and menstruation, based on comparison result between the time series values of the body temperature and menstruation data and the numerical range of the medical reference information corresponding to the body temperature and the menstruation, and determines a good state when the value of the body temperature or the menstruation is within the numerical range of the medical reference information, and determines an improved state when the value of the body temperature or the menstruation varies and approaches the numerical range of the medical reference information, and
the output unit outputs a time series graph of the body temperature and menstruation data.

3. The health care system according to claim 1,
wherein the analysis unit determines the health state of each user, including good or bad of a combination of values of a plurality of examination items and relative improvement or deterioration in time series of the combination of the values of the plurality of examination items, based on comparison result between time series values of the plurality of examination items of the examination result data and the numerical range of the medical reference information corresponding to each of the plurality of examination items, and
the analysis unit determines the good state when a first value of the examination item is within a first numerical range and a second value of the examination item is within a second numerical range, and determines the improved state when the first value of the examination item varies and approaches the first numerical range and the second value of the examination item varies and approaches the second numerical range.

4. The health care system according to claim 2,
wherein the data management unit registers and manages the health information which includes information including basal body temperature and a menstruation date and information on a symptom, which are input by the user and serve as the body temperature and menstruation data,
the analysis unit uses the basal body temperature, the menstruation date, and the symptom to calculate predetermined evaluation item values including a menstrual cycle, a low temperature phase, a high temperature phase, a temperature difference between the low temperature phase and the high temperature phase, an increase or decrease in a frequency of the symptom, and a predicted ovulation date, calculates time series variations of the evaluation item values, and determines improved or deteriorated states of the evaluation item values as the health state of each user based on comparison between the variations of the evaluation item values and predetermined values, and
the output unit outputs information including determination results of the evaluation item values.

5. The health care system according to claim 1,
wherein the data management unit registers and manages user input information in time series, which is input into a calendar date based on the operation of the user and includes action of the user,
the analysis unit extracts information on life habits which include past action of the user assumed to be relevant to a present health state of each user,
the analysis unit extracts information including at least one of a life habit which includes action assumed to be medically relevant and a life habit which includes frequent action in a past period, and
the output unit outputs a message including the extracted information on the life habits which include the action.

6. The health care system according to claim 1,
wherein the data management unit registers and manages user input information in time series, which is input into a calendar date based on the operation of the user and includes action of the user,
the analysis unit determines a tendency of a change in the action in time series in the user input information, and
the output unit outputs a message indicating the tendency of the change in the action of the user.

7. The health care system according to claim 1,
wherein the data management unit registers and manages user input information in time series, which is input into a calendar date based on the operation of the user and includes a symptom of the user,
the analysis unit determines a tendency of a change in the symptom in time series in the user input information, and
the output unit outputs a message indicating the tendency of the change in the symptom of the user.

8. The health care system according to claim 1,
wherein the data management unit registers and manages user input information in time series, which is input into a calendar date based on the operation of the user and includes arbitrary text of the user,
the analysis unit analyzes and extracts a word included in the text in the user input information and determines the health state of each user, including a positive or a negative state of a symptom and a feeling, based on the analysis result of the word, and
the output unit outputs a message including the analysis result of the word.

9. The health care system according to claim 1,
wherein the data management unit registers and manages the examination result data including hormones including a plurality of types of female hormones serving as a plurality of examination items of examination including a blood test, and
the analysis unit determines the health state of each user based on comparison result between values of the hormones including the plurality of types of female hormones and the numerical range of the medical reference information corresponding to each of the hormones including the female hormones.

10. The health care system according to claim 2,
wherein the analysis unit determines, as the health state of each user, possibilities of diseases including a plurality of diseases concerned with obstetrics, gynecology, and reproductive medicine based on comparison result between the health information, which includes, as elements, values of a plurality of examination items of the examination result data and the values of the body temperature and menstruation data, and the numerical range of the medical reference information,
the numerical range of the medical reference information includes threshold values for the elements for each of the diseases, and
the output unit outputs a message including a warning for the possibilities of the diseases.

11. The health care system according to claim 2,
wherein the analysis unit determines, as the health state of each user, a state of ease of pregnancy or a possibility of infertility based on comparison result between the health information, which includes, as elements, values of a plurality of examination items of the examination result data and the values of the body temperature and menstruation data, and the numerical range of the medical reference information,
the numerical range of the medical reference information includes threshold values for the elements, and
the output unit outputs a message including the state of the ease of the pregnancy or the possibility of the infertility.

12. The health care system according to claim 1,
wherein the output unit outputs, as an output message appropriate for the health state of each user, information including explanation of a state of the tendency, medical knowledge, medical advice, consultation recommendation for treatment or examination, recommended action, or a recommended product.

13. The health care system according to claim 1,
wherein the medical examination information includes setting of a numerical range of system-unique reference information associated with the numerical range of the medical reference information,
the analysis unit determines the health state of each user, based on comparison result between the time series values of the examination item of the examination result data of the user and the numerical range of the system-unique reference information corresponding to the examination item and the medical institution or the examination institution used by the user, and
the numerical range of the system-unique reference information is set to a numerical range covering a plurality of pieces of medical reference information by using OR condition, AND condition, statistical values, or arbitrary set values in numerical ranges of the plurality of pieces of medical reference information of a plurality of medical institutions or examination institutions.

14. The health care system according to claim 2,
wherein the terminal of the user inputs the examination result data or the body temperature and menstruation data from an external medical device or an external terminal via communication and transmits the data to the server device to be registered.

15. The health care system according to claim 2,
wherein the terminal of the user or the server device inputs the examination result data or the body temperature and menstruation data by recognizing voice of the user and registers the data in the server device.

16. The health care system according to claim 2,
wherein the data management unit includes a graph interpolation unit,
the graph interpolation unit creates an interpolation graph, which is easy to be seen, by interpolating values in non-registered dates between values in registered dates in the examination result data or the body temperature and menstruation data of each user, and
the output unit outputs information including the interpolation graph.

17. The health care system according to claim 2,
wherein the data management unit sets a medical reference graph or a system-unique reference graph relating to the examination result data or the body temperature and menstruation data,
the analysis unit includes a graph matching unit,
the graph matching unit determines a degree of similarity by comparing a graph of the examination result data or the body temperature and menstruation data of the user with the reference graph corresponding to the graph, and
the output unit outputs information including the graph of the user, the reference graph, and the degree of similarity.

18. The health care system according to claim 1,
wherein the server device includes a relevant information search unit,
the relevant information search unit automatically searches for relevant information on the Internet by setting, as a search condition, a word included in user input information of the user, processing definition information used for processing of the determination, information on a result of the determination, or output information including the message, and provides the terminal of the user with the relevant information obtained as a result of searching.

19. The health care system according to claim 1,
wherein the server device includes a partner management unit, and
the partner management unit registers and manages a female user and a male user as partners, displays information on the male user of the partners on a screen of the terminal of the female user, and displays information on the female user of the partners on a screen of the terminal of the male user.

20. The health care system according to claim 19,
wherein the partner management unit automatically notifies the screen of the terminal of the user of the partners, of information in a predetermined item concerned with the health state of the female user or the male user and displays the information on the screen according to the user setting.

21. The health care system according to claim 19,
wherein the server device includes a pregnancy support unit, and
as processing for supporting pregnancy activity of the female user and the male user of the partners, the pregnancy support unit displays, on the screen of the terminal of the female user, information including a message for activating the pregnancy activity appropriate for the health state of the male user and displays, on the screen of the terminal of the male user, information including a message for activating the pregnancy activity appropriate for the health state of the female user.

22. The health care system according to claim 21,
wherein the pregnancy support unit generates, as the message for activating the pregnancy activity, coaching information including advice on action for the male user to act on the female user, and displays the coaching information on the screen of the terminal of the male user.

23. The health care system according to claim 21,
wherein the pregnancy support unit calculates an index value representing fertility according to the health state of the female user of the partners, calculates an index value representing fertility according to the health state of the male user of the partners, calculates an index value of the pregnancy activity of the male and female partners by using the index value of the female user and the index value of the male user, and displays information including the index value.
